(19) United States
(12) Patent Application Publication
(10) Pub. No.: US 2003/0135888 A1
(43) Pub. Date: Jul. 17, 2003

(54) Genes that are modulated by posttranscriptional gene silencing

(76) Inventors: Tong Zhu, San Diego, CA (US); Xun Wang, San Diego, CA (US); Hur-Song Chang, San Diego, CA (US); Steven P. Briggs, Del Mar, CA (US); Bret Cooper, La Jolla, CA (US); Jane Glazebrook, San Diego, CA (US); Stephen A. Goff, Encinitas, CA (US); Fumiaki Katagiri, San Diego, CA (US); Joel Kreps, Carlsbad, CA (US); Todd Moughamer, San Diego, CA (US); Nicholas Provart, Toronto (CA); Darrell Ricke, San Diego, CA (US)

Correspondence Address: TORREY MESA RESEARCH INSTITUTE, INTELLECTUAL PROPERTY DEPARTMENT, 3115 MERRYFIELD ROW, SAN DIEGO, CA, 92121, US

(21) Appl. No.: 10/259,165

(22) Filed: Sep. 26, 2002

Related U.S. Application Data
(60) Provisional application No. 60/368,327, filed on Mar. 27, 2002.
(60) Provisional application No. 60/325,277, filed on Sep. 26, 2001.
(60) Provisional application No. 60/370,620, filed on Apr. 4, 2002.

(51) Int. Cl. A01H 5/00; C07H 21/04; C12N 15/82; C12N 5/04

(52) U.S. Cl. 800/288; 536/23.2; 435/419; 800/320; 800/306; 800/320.1; 800/320.2; 800/320.3; 435/320.1

(57) ABSTRACT

The invention provides a method to identify genes that are modulated by posttranscriptional gene silencing as well as regulatory elements and methods to modulate gene expression by posttranscriptional gene silencing.

### CROSS-REFERENCE TO RELATED APPLICATIONS

This application claims the benefit of U.S. Provisional Application No. 60/368,327, filed Mar. 27, 2002, U.S. Provisional Application No. 60/325,277 filed Sep. 26, 2001, and U.S. Provisional Application No. 60/370,620 filed Apr. 4, 2002, each of which is incorporated herein by reference in its entirety.

### REFERENCE TO MATERIAL SUBMITTED ON COMPACT DISC

The sequence listing accompanying this application is contained on compact disc. The material on the CD-ROM (filed in duplicate herewith), on CD volume labled “Copy 1”and “Copy 2”, each containing a text file named “70030NP, SEQ, LST” created Sep. 26, 2002, having a size of 1.36 MB, is hereby incorporated by reference in its entirety pursuant to 37 C.F.R. §1.52(e)(5).

### FIELD OF THE INVENTION

The present invention generally relates to the field of plant molecular biology, and more specifically to the regulation of gene expression within a cell by posttranscriptional gene silencing.

### BACKGROUND OF THE INVENTION

Plant gene silencing was originally thought to be a quirk of transformation procedures, but is now recognized to be a facet of vitally important gene regulatory systems present in all organisms. Posttranscriptional gene silencing (PTGS) refers to the trans-activation of homologous genes due to increased RNA degradation. This is a stable, reversible epigenetic modification due to increased RNA degradation triggered by sequence-specific signals that, in some cases, can spread systemically. Although first described in transgenic plants, it is now recognized that very similar forms of PTGS occur in a wide variety of organisms.

Posttranscriptional gene silencing (PTGS) in plants is analogous to RNA interference (RNAi), first identified in C. elegans, but now known to function in several other animals, including insects, vertebrates and cnidarian. The few existing reports of PTGS in monocots suggest that it probably operates through mechanisms similar to those observed in dicots since shared features include the existence and triggering of coding region methylation (Ingelbrecht et al., Proc. Natl. Acad. Sci. USA, 90:10502 (1994) and Ingelbrecht et al., Plant Physiol., 119:1187 (1999)), mitotic stability (Guo et al., Mol. Plant-Microbe Interact., 12:103 (1999) and Ingelbrecht et al., Plant Physiol., 119:1187 (1999)), the induction of virus recovery in transgenic plants (Ingelbrecht et al., Plant Physiol., 119:1187 (1999) and Pinto et al., Nature Biotechnol., 17:702 (1999)), and the ability to cosuppress endogenous genes (Yin et al., EMBO J., 16:5247 (1997)). Many of the host genes that are responsible for PTGS-like processes in C. elegans (RNAi) and Neurospora (quelling) are conserved in plants, suggesting that features of PTGS are common between widely diverged groups of eukarya (Cogoni et al., Nature, 399:166 (1999); Cogoni et al., Science, 286:2342 (1999); Ketting et al., Cell, 90:133 (1999); Tabara et al., Cell, 99:123 (1999)).

Although there are reports of PTGS induction with single-copy inserts, the presence of inverted repeats and multiple copies of transgenes are typically associated with silencing (Jorgensen et al., Plant Mol. Biol., 31:957 (1996)). In general, PTGS is correlated with active transcription of the transgene, and transcriptional silencing of the transgene has been shown to reverse PTGS (English et al., Plant J., 12:597 (1997) and Que et al., Dev. Genet., 22:100 (1998)).

It is believed that either ectopic DNA-DNA or DNA-RNA pairing, or the formation (intended or unintended) of antisense transcripts that give rise to dsRNA from cryptic promoters 3′ to the transgene insert, results in the formation of aberrant RNA transcripts (which include RNAs lacking polyadenylation, or short polyadenylated RNAs, generated as a result of incomplete transcription) that activate silencing (Baulcombe et al. Curr. Opin. Biotechnol., 7:173 (1996); Depicker et al., Curr. Opin. Cell Biol., 9:373 (1997); Metzlaff et al., Cell, 88:845 (1997); Montgomery et al., Trends Genet., 14:255 (1998); Que et al., Dev. Genet., 22:100 (1998); Stam et al., Mol. Cell Biol., 18:6165 (1998); and Wassenegger et al., Plant Mol. Biol., 37:349 (1998)). High levels of transcription, giving rise to accumulation of normal transcripts that exceeds a ‘threshold’ level, have been proposed to activate silencing (Lindbo et al., Plant Cell, 5:1749 (1993)). Additionally, small RNAs have been shown to trigger PTGS in Drosophila preparations (Elbashir et al., Genes Devel., 15:188 (2001)). Thus, normal transcripts, antisense transcripts and aberrant transcripts can all give rise to PTGS.

Upon the entry of PTGS-eliciting RNA into the cytoplasm, its degradation and that of any homologous RNAs is postulated to ensue and several reports have shown the presence of either degradation intermediates or aberrant transcripts (Goodwin et al., Plant Cell, 8:95 (1996) and van Eldik et al., Nucl. Acids Res., 26:5176 (1998)). A critical question is how these RNAs are distinguished from normal cellular RNAs and targeted for elimination. A possibility is that host RNA-dependent RNA polymerase (RdRP) and RNase functions are active during these surveillance events. The dsRNA formed through RdRP activity presumably serves as a target for RNase, providing the basis for sequence specificity of degradation. Small (25 nt) fragments of antisense orientation to the elicitor RNA have been observed in all studied cases of PTGS in plants (Hamilton et al., Science, 286:950 (1999)), but it is still not certain if host RdRP is involved in their synthesis.

Formation of complementary RNA by RdRP activity appears to be followed by the degradation of dsRNAs by an RNase which may be constitutive or specific to PTGS (Lindbo et al., Plant Cell, 5:1749 (1993); D. Baulcombe, Plant Mol. Biol., 32:79 (1996)).

Mutants that increase or decrease the severity of PTGS have been isolated in Arabidopsis and PTGS-related genes have been cloned (Elmayan et al., Plant Cell, 10:1747 (1998); Morel et al., Plant Mol. Biol., 43:275 (2000); Dalmay et al., Cell, 101:543 (2000); Fagard et al., Proc. Natl. Acad. Sci. USA, 97:11650 (2000); Mourrain et al., Cell, 101:533 (2000)) and some viral proteins have been shown to be capable of reversing PTGS (Anandalakshmi et al., Proc. Natl. Acad. Sci. USA, 95:13079 (1998); Beclin et al., Virology, 252:313 (1998); Brigneti et al., EMBO J., 17:6739 (1998); Kasschau et al., Cell, 95:461 (1998); Marathe et al., Plant Mol. Biol., 2000). Both DNA and RNA viruses, and viruses that affect monocots and dicots have been shown to possess these proteins.

Gene silencing has been shown to occur in instances where there has been an insertion of a single copy of a gene. This situation is of great concern as it may cause a reduction in the production of a desired gene product. Presently there is little insight into how or why such sequences are targeted, although it is clear that in many instances excessive expression levels lead to PTGS. Also, little insight exists into the stochastic processes that lead to silencing in some lines while sibling progeny with an apparently identical genomic complement and organization express transgenic information reliably and at high levels.

Stability of expression is vital for future increases in performance of major crops, especially the grasses, which play a foremost role in the agricultural economy of all nations. Biotechnological manipulation offers great potential to provide for many improvements, including disease resistance and nutritional and processing qualities as well as abiotic stress tolerance and overall yield enhancement. The ability to control PTGS would allow for the increased production of a desired gene product or the ability to decrease or eliminate the expression of an undesired gene product. Thus, what is needed is a method to identify genes involved in PTGS in order that these genes may be regulated to either increase or decrease expression of a target gene. Also, the ability to control PTGS would allow for increased production of a desired product by overcoming gene silencing due to PTGS.

### SUMMARY OF THE INVENTION

The present invention relates to clusters or groups of polynucleotides from cereals, but especially from rice, the expression of which is altered in response to posttranscriptional gene silencing (PTGS).

The use of microarray technology has allowed the identification of such clusters of polynucleotides which are modulated within a cell by posttranscriptional gene silencing in Arabidopsis thaliana.

Provided herein are homologs and orthologs of such polynucleotide sequences in other plant species and in particular cereals. Thus, the invention provides polynucleic acid segments corresponding to genes modulated within a cell by posttranscriptional gene silencing from cereals, and homologs and orthologs thereof; variants of such sequences, and nucleotide sequences encoding substantially similar cereal PTGS-regulated polypeptides expressed therefrom.

In particular, the invention provides orthologs from cereal species to the nucleic acid segments corresponding to the nucleic acid sequences disclosed in international application no PCT/EP02/03806, filed Apr. 05, 2002 the disclosure of which is incorporated herein by reference in its entirety, and listed in sequence identifiers numbered 1-251, and the corresponding genes. These orthologs may be determined through visual insepection, mechanical and electronic means, such as a BLAST search.

The orthologs are nucleic acid sequences that encode polypeptides that are substantially similar to polypeptides encoded by any of the nucleic acid sequences listed in sequence identifiers numbered 1-251 of international application no PCT/EP02/03806, and the corresponding genes. Preferably the ortholog nucleic acid sequences have a similarity value less than 1×10⁻⁵ to the corresponding nucleic acid sequence selected from the nucleic acid sequences listed in sequence identifiers numbered 1-251 of international application no PCT/EP02/03806, and the corresponding genes. Preferably the similarity value is less than 1×10⁻¹⁰. More preferably the similarity value is less than 1×10⁻²⁰. More preferably the similarity value is less than 1×10⁻²⁵. Most preferably, the nucleic acid sequences are those identified in SEQ ID NOs: 1 to 342.

The invention additionally provides an expression cassette containing a regulatory sequence operably linked to a nucleic acid segment that is modulated within a cell by posttranscriptional gene silencing. A cell containing the expression cassette is provided. A construct containing a vector and an expression cassette, which has a regulatory sequence operably linked to a polynucleic acid segment of the invention, is provided. A cell containing a construct, having an expression cassette within a vector, is also provided. A mutagenesis cassette having an intervening nucleic acid sequence linked on both ends to a flanking nucleic acid sequence, which hybridizes under low stringency conditions to a gene that is modulated within a cell by posttranscriptional gene silencing is provided. Also provided is a construct that contains a vector and a mutagenesis cassette. Polypeptides are provided that are encoded by the polynucleic acid segments of the invention and variants thereof. The invention also provides a method to isolate a regulatory element that modulates the expression of a gene within a cell by posttranscriptional gene silencing. Accordingly, a regulatory element that modulates expression of a gene within a cell by posttranscriptional gene silencing is provided. An expression cassette containing a regulatory element that modulates gene expression by posttranscriptional gene silencing is provided. A method to create a mutant cell using the mutagenesis cassette of the invention is provided. A method to augment the genome of a plant with a polynucleic acid segment of the invention is provided. The seeds, fruit, and other products of the augmented plant are also provided. Transgenic plants containing the nucleic acid segments of the invention and that products of the transgenic plants are provided. A method to identify an expression product that interacts with an expression product that is modulated within a cell by posttranscriptional gene silencing is provided. Also provided is a method to modulate the expression of a gene by posttranscriptional gene silencing. Further provided are ortholog polynucleic acid sequences that hybridize under low stringency conditions to, to that encode polypeptides that are substantially similar to the nucleic acid segments of the invention. Also provided are cells and plants transformed with the orthologs of the invention as well as transgenic plants and the products thereof. The invention also provides methods to shuffle the nucleic acid segments of the invention to encode polypeptides that exhibit altered activity relative to the corresponding native polypeptide. Also provided is a computer readable medium containing the nucleic acid sequences of the invention as well as methods for use of the computer readable medium.

The methods to identify expression products modulated within a cell by posttranscriptional gene silencing involve extracting the expression products from two related cells that exhibit differences in posttranscriptional gene silencing and then comparing the products. This comparison allows for the determination of expression products that are up-modulated or down-modulated by posttranscriptional gene silencing. Preferably one of the related cells has posttranscriptional gene silencing while the other cell does not. Preferably the expression product is a polypeptide. More preferably the expression product is a transcription product. Most preferably the expression product is a messenger RNA. Preferably the cell is a plant cell. More preferably the cell is an embryophyte cell. Even more preferably the cell is an spermatophyte cell. Still even more preferably the cell is a eudicotyledon. Much more preferably the cell is a Brassicales cell. Most preferably the cell is from Arabidopsis. Preferably the cell is a cereal plant cell. More preferably the plant cell is from a plant that is grown for food. Most preferably the cell is from a plant that is commercially grown.

The present invention provides isolated polynucleic acid segments having at least 70%, preferably 80%, more preferably 90% and even more preferably 98% identity to the nucleic acid sequences corresponding to genes modulated within a cell by posttranscriptional gene silencing from cereals, but especially from rice, identified in Tables 1 and 2 and listed in the sequence listing, or the complement thereof. Also provided are variants of the nucleic acid sequences listed in the sequence listing. Most preferred embodiments include isolated polynucleic acid segments having at least 99% identity therewith. Preferred embodiments also include single unit percentage identities based upon these classes. For example, 71%, 72%, 73% and the like, up through at least the 99% class are preferred. A polynucleic acid segment according to the invention can be DNA or RNA. Preferably the isolated polynucleic acid segments modulate posttranscriptional gene silencing within a cell. Preferably a polynucleic acid segment of the invention is contained within a vector. More preferably the polynucleic acid segment of the invention is contained within a plasmid, phagemid, cosmid, virus, F-factor or phage. Most preferably the polynucleic acid segment of the invention is contained within a Ti plasmid.

One embodiment provides an isolated polynucleotide comprising a plant nucleotide sequence: (a) selected from the group consisting of (a) SEQ ID NOs: 1 to 341 or a fragment thereof which encodes a partial-length polypeptide having substantially the same activity as the full-length polypeptide; (b) having substantial similarity to (a); (c) having at least 15, at least 20, or at least 30 nucleotides and capable of hybridizing to (a) or the complement thereof under stringent, highly stringent or very highly conditions; (d) having at least 15, at least 20, or at least 30 nucleotides and capable of hybridizing to a nucleic acid comprising 50 to 200 or more consecutive nucleotides of a nucleotide sequence given in SEQ ID NOs: 1 to 341 or the complement thereof under stringent, highly stringent or very highly stringent conditions; (e) a sequence complementary to (a), (b) or (c); or (f) a sequence which is a reverse complement of (a), (b) or (c).

The invention also provides a construct containing a vector and a polynucleic acid segment having at least 70%, preferably 80%, more preferably 90% and even more preferably 98% identity to the nucleic acid sequences corresponding to genes modulated within a cell by posttranscriptional gene silencing from cereals, but especially from rice, identified in Tables 1 and 2 and listed in the sequence listing, or the complement thereof. Most preferred embodiments include polynucleic acid segments having at least 99% identity therewith. Preferred embodiments also include single unit percentage identities based upon these classes. For example, 71%, 72%, 73% and the like, up through at least the 99% class are preferred. Also included are variants of the cereal polynucleic acid sequences listed in the sequence listing. Preferably the vector is a plasmid, phagemid, cosmid, virus, F-factor or phage.

The invention provides an expression cassette containing a regulatory sequence operably linked to a polynucleic acid segment having at least 70%, preferably 80%, more preferably 90% and even more preferably 98% identity to the nucleic acid sequences corresponding to genes modulated within a cell by posttranscriptional gene silencing from cereals, but especially from rice, identified in Tables 1 and 2 and listed in the sequence listing, or the complement or variant thereof. Most preferred embodiments include cereal polynucleic acid segments having at least 99% identity therewith. Preferred embodiments also include single unit percentage identities based upon these classes. For example, 71%, 72%, 73% and the like, up through at least the 99% class are preferred. The expression cassette can also contain the polynucleic acid segment in an anti-sense orientation relative to the regulatory sequence. Preferably the regulatory sequence contains a promoter, operator, enhancer, repressor binding site and/or a transcription factor binding site. More preferably the regulatory sequence is a promoter. Most preferably the regulatory sequence is an inducible promoter. Preferably the expression cassette is contained within a cell. More preferably the expression cassette is contained within the genome of a cell. Most preferably the expression cassette is contained within the genome of a transgenic organism. Preferably the cell is a plant cell. More preferably the cell is an embryophyte cell. Even more preferably the cell is an spermatophyte cell. Still even more preferably the cell is a eudicotyledon. Much more preferably the cell is a cereal cell. Most preferably the cell is from rice. Preferably the cell is a cereal plant cell. More preferably the plant cell is from a plant that is grown for food. Most preferably the cell is from a plant that is commercially grown.

A construct containing a vector and an expression cassette is provided. Preferably the vector contained within the construct is a plasmid, cosmid, phagemid, virus, F-factor or phage. More preferably the vector contained within the construct is a virus. Most preferably the vector contained within the construct is a Ti plasmid. Preferably the construct is contained with a cell. More preferably the construct is contained within a plant cell. Even more preferably the cell is a cereal plant cell. Still even more preferably the cell is from a plant that is grown for food. Most preferably the cell is from a plant that is commercially grown. Preferably the plant cell is an embryophyte cell. Even more preferably the plant cell is an spermatophyte cell. Still even more preferably the plant cell is a eumonocotyledon. Much more preferably the plant cell is a cereal cell. Most preferably the plant cell is from rice.

The invention provides a mutagenesis cassette containing an intervening nucleic acid sequence linked on both ends to a flanking nucleic acid sequence that hybridizes under low stringency conditions to a gene that is modulated by posttranscriptional gene silencing. Preferably the intervening nucleic acid sequence is a selectable marker. More preferably the intervening nucleic acid sequence is a selectable marker for chemical resistance. Preferably the intervening nucleic acid sequence is linked on both ends to a flanking nucleic acid,sequence which hybridizes to a cereal nucleic acid sequence as listed in the sequence listing. More preferably the intervening nucleic acid sequence is linked on both ends to a flanking nucleic acid sequence that hybridizes to a nucleic acid sequence that modulates gene expression by posttranscriptional gene silencing. Preferably the mutagenesis cassette is contained within a vector. More preferably the mutagenesis cassette is contained within a cosmid, plasmid, phagemid, virus, phage, F-factor or Ti plasmid. Most preferably the mutagenesis cassette is contained within a Ti plasmid.

The invention also provides a polypeptide encoded by a nucleic acid sequence having at least 70%, preferably 80%, more preferably 90% and even more preferably 98% identity to the nucleic acid sequences corresponding to genes modulated within a cell by posttranscriptional gene silencing from cereals, but especially from rice, identified in Tables 1 and 2 and listed in the sequence listing. Most preferred embodiments include isolated polynucleic acid segments having at least 99% identity therewith. Preferred embodiments also include single unit percentage identities based upon these classes. For example, 71%, 72%, 73% and the like, up through at least the 99% class are preferred. Preferably the polypeptide of the invention modulates gene expression within a cell by posttranscriptional gene silencing.

Thus, a method to modulate gene expression by posttranscriptional gene silencing is provided by the invention. The method involves transforming a cell with a polynucleic acid segment that modulates gene expression by posttranscriptional gene silencing. Preferably the polynucleic acid segment is contained within an expression cassette. More preferably the polynucleic acid segment is included within a construct containing a vector and the polynucleic acid segment. Most preferably the polynucleic acid segment is contained within an expression cassette that is integrated into the chromosome of a cell. Preferably the cell is a plant cell. More preferably the cell is a cereal plant cell. Even more preferably the cell is from a plant that is grown for food. Most preferably the cell is from a plant that is commercially grown. Preferably the plant cell is an embryophyte cell. Even more preferably the plant cell is a spermatophyte cell. Still even more preferably the plant cell is a eudicotyledon. Much more preferably the plant cell is a cereal cell. Most preferably the plant cell is from rice.

A method to isolate a regulatory element that regulates expression of a polynucleic acid segment within a cell by posttranscriptional gene silencing is provided. The method involves hybridizing an oligonucleotide probe to a polynucleic acid segment that is regulated within a cell by posttranscriptional gene silencing. The polynucleic acid segment is contained within a polynucleic acid fragment that also contains the regulatory element. The complex containing the hybridized probe is then isolated to obtain the regulatory element. The nucleic acid within the formed complex may be sequenced to determine the nucleotide sequence of the regulatory element. Preferably the oligonucleotide probe is constructed from a sequence that hybridizes under low stringency conditions to a nucleic acid sequence corresponding to genes modulated within a cell by posttranscriptional gene silencing from cereals identified in Tables 1 and 2 and listed in the sequence listing or the complement thereof. Preferably the cell is a plant cell. More preferably the cell is a cereal plant cell. Most preferably the cell is from a plant that is grown commercially. Preferably the cell is a plant cell. Preferably the plant cell is an embryophyte cell. More preferably the plant cell is a spermatophyte cell. Even more preferably the plant cell is a eumonocotyledon. Much more preferably the plant cell is a cereal cell. Most preferably the plant cell is from rice.

Another method to obtain a regulatory element that modulates expression of a polynucleic acid segment within a cell by posttranscriptional gene silencing is provided. The method involves hybridizing an oligonucleotide primer to an open reading frame having expression that is modulated by posttranscriptional gene silencing. The open reading frame is contained within nucleic acid extracted from a cell that also contains the regulatory element that controls expression of the open reading frame. A second oligonucleotide primer is annealed to the nucleic acid extracted from the cell in a position that is 5′ or 3′ to the open reading frame. A polymerase chain reaction is conducted to amplify the nucleic acid located between the two primers. This amplified nucleic acid may be isolated to obtain the regulatory element. The amplified nucleic acid may also be sequenced to determine the nucleotide sequence of the regulatory element. Preferably the second oligonucleotide primer anneals to the nucleic acid extracted from the cell in a position that is 5′ to the open reading frame that is regulated within the cell by posttranscriptional gene silencing. Preferably the cell is a plant cell. More preferably the cell is a cereal plant cell. Most preferably the cell is from a plant that is commercially grown. Preferably the cell is a plant cell. Preferably the plant cell is an embryophyte cell. More preferably the plant cell is a spermatophyte cell. Even more preferably the plant cell is a eumonocotyledon. Much more preferably the plant cell is a cereal cell. Most preferably the plant cell is from rice. Preferably the nucleic acid extracted from the cell is from a chloroplast. More preferably the nucleic acid extracted from the cell is genomic DNA. Preferably the first oligonucleotide primer has at least 70%, preferably 80%, more preferably 90% and even more preferably 98% identity to the nucleic acid sequences corresponding to genes modulated within a cell by posttranscriptional gene silencing from cereals, but especially from rice, identified in Tables 1 and 2 and listed in sequence listing, or the complement thereof. Most preferably the first oligonucleotide primer has at least 99% identity therewith. Preferred embodiments also include single unit percentage identities based upon these classes. For example, 71%, 72%, 73% and the like, up through at least the 99% class. Preferably the second oligonucleotide primer is a degenerate primer. More preferably the second oligonucleotide primer is a multiplicity of degenerate primers. Preferably thermal asymmetric interlaced polymerase chain reaction is used to amplify the nucleic acid between the first and second primers. Accordingly, the invention also provides a regulatory element that modulates expression of an open reading frame by posttranscriptional gene silencing.

The invention also provides an expression cassette having a regulatory element that modulates expression of an operably linked open reading frame by posttranscriptional gene silencing. Preferably the expression cassette is included within a vector to form a construct. More preferably the construct containing the expression cassette and a vector is contained within a cell. Preferably the cell is a plant cell. More preferably the cell is a plant cell that is grown into a transgenic plant. Most preferably the cell is a plant cell that is grown into a commercial transgenic plant. Preferably the plant cell is an embryophyte cell. More preferably the plant cell is an spermatophyte cell. Even more preferably the plant cell is a eumonocotyledon. Much more preferably the plant cell is a cereal cell. Most preferably the plant cell is from rice. Preferably the plant cell is a cereal plant cell. More preferably the plant cell is from a plant that is grown for food. Most preferably the cell is from a plant that is commercially grown.

The invention also provides a method to augment the genome of a cell that includes contacting the nucleic acid within a cell with a polynucleic acid segment of the invention and growing the cell. Preferably the cell is a plant cell. More preferably the cell is a plant cell that is grown into a transgenic plant. Most preferably the cell is from a commercial plant and is grown into a transgenic plant. Preferably the plant cell is an embryophyte cell. More preferably the plant cell is an spermatophyte cell. Even more preferably the plant cell is a eumonocotyledon. Much more preferably the plant cell is a cereal cell. Most preferably the plant cell is from rices. Preferably the plant cell is a cereal plant cell. More preferably the plant cell is from a plant that is grown for food. Most preferably the cell is from a plant that is commercially grown. The polynucleic acid segment has at least 70%, preferably 80%, more preferably 90% and even more preferably 98% identity to the nucleic acid sequences corresponding to genes modulated within a cell by posttranscriptional gene silencing from cereals identified in Tables 1 and 2 and listed in the sequence listing. Most preferred embodiments include polynucleic acid segments having at least 99% identity therewith. Preferred embodiments also include single unit percentage identities based upon these classes. For example, 71%, 72%, 73% and the like, up through at least the 99% class. The invention also provides the seeds, fruits and other products of the augmented plant. The invention also provides a transgenic organism having a genome that contains the nucleic acid segments of the invention and the seeds, fruits, and other products thereof. Preferably the transgenic organism is a plant. More preferably the transgenic plant cell is an embryophyte. More preferably the transgenic plant is a spermatophyte. Even more preferably the transgenic plant is a eumonocotyledon. Much more preferably the transgenic plant is a cereal cell. Most preferably the transgenic plant is rice. Preferably the transgenic plant is a cereal plant cell. More preferably the transgenic plant is from a plant that is grown for food. Most preferably the transgenic plant is commercially grown.

The invention also provides a method of using the mutagenesis cassette to create a mutation in a cell. The method includes the steps of contacting the mutagenesis cassette with the nucleic acid within a cell to yield a cell having a mutation in a gene that is modulated by posttranscriptional gene silencing. Preferably the cell is a plant cell. More preferably the cell is a plant cell that is grown into a transgenic plant. Most preferably the cell is a plant cell that is grown into a commercial transgenic plant. Preferably the plant cell is an embryophyte cell. More preferably the plant cell is an spermatophyte cell. Even more preferably the plant cell is a eumonocotyledon. Much more preferably the plant cell is a cereal cell. Most preferably the plant cell is from nice. Preferably the plant cell is a cereal plant cell. More preferably the plant cell is from a plant that is grown for food. Most preferably the cell is from a plant that is commercially grown.

A method to identify a first expression product that interacts with an expression product modulated within a cell by posttranscriptional gene silencing is provided. The method involves contacting an expression product that is modulated with the first expression product to form a detectable complex and identifying the first expression product by separating the detectable complex. Preferably the expression product that is modulated is encoded by a nucleic acid sequence having at least 70%, preferably 80%, more preferably 90% and even more preferably 98% identity to the nucleic acid sequences corresponding to genes modulated within a cell by posttranscriptional gene silencing from cereals identified in Tables 1 and 2 and listed in the sequence listing, and the corresponding genes. Most preferred embodiments include expression products encoded by polynucleic acid segments having at least 99% identity therewith. Preferred embodiments also include single unit percentage identities based upon these classes. For example, 71%, 72%, 73% and the like, up through at least the 99% class. Preferably the nucleic acid sequence encoding the expression product that is modulated is used within a yeast two-hybrid system. Preferably the nucleic acid sequence encoding the expression product that is modulated is fused to a marker polypeptide that allows detection of the complex formed between the first expression product and the expression product that is modulated. Preferably the marker polypeptide is an epitope for an antibody. More preferably the marker polypeptide is glutathione S-transferase.

The invention provides orthologs from cereal species to the nucleic acid segments corresponding to the nucleic acid sequences listed in sequence identifiers numbered 1 to 342, and the corresponding genes. These orthologs may be determined through visual insepection, mechanical and electronic means, such as a BLAST search. The orthologs may be nucleic acid sequences that hybridize under low stringency conditions to any of the nucleic acid sequences listed in sequence identifiers numbered 1 to 342, and the corresponding genes. The orthologs may also be nucleic acid sequences that encode polypeptides that are substantially similar to polypeptides encoded by any of the nucleic acid sequences listed in sequence identifiers numbered 1 to 342, and the corresponding genes. Preferably the ortholog nucleic acid sequences have a similarity value less than 1×10⁻⁵ to the corresponding nucleic acid sequence selected from the nucleic acid sequences listed in sequence identifiers numbered 1 to 342, and the corresponding genes. More preferably the similarity value is less than 1×10⁻¹⁰. Even more preferably the similarity value is less than 1×10⁻²⁰. Most preferably the similarity value is less than 1×10²⁵.

Accordingly, the invention provides cells transformed with orthologs corresponding to a nucleic acid sequence listed in sequence identifiers numbered 1 to 342, and the corresponding genes. The invention also provides transgenic organisms containing the indicated ortholog sequences and the products thereof. The nucleic acid segments corresponding to the orthologs may be introduced into the cells, plants, and organisms according to methods known in the art and as indicated herein. Preferably the cell is a plant cell. More preferably the cell is a plant cell that is grown into a transgenic plant. Most preferably the cell is a plant cell that is grown into a commercial transgenic plant. Preferably the plant cell is an embryophyte cell. More preferably the plant cell is an spermatophyte cell. Even more preferably the plant cell is a eumonocotyledon. Much more preferably the plant cell is a cereal cell. Most preferably the plant cell is from rice. Preferably the plant cell is a cereal plant cell. More preferably the plant cell is from a plant that is grown for food. Most preferably the cell is from a plant that is commercially grown.

A method to shuffle the nucleic acids of the invention is provided. This method involves fragmentation of a nucleic acid corresponding to a nucleic acid sequence listed in sequence identifiers numbered 1 to 342; 473 to 539; 540 to 673, and 674 to 779, respectively, orthologs, and the corresponding genes, followed by religation. This method allows for the production of polypeptides having altered activity relative to the native form of the polypeptide. Preferably the cell is a plant cell. More preferably the plant cell is a cereal plant cell. Most preferably the cell is a plant cell that is commercially grown. Accordingly, the invention provides transgenic plants containing nucleic acid segments produced through shuffling that encode polypeptides having altered activity relative to the corresponding native polypeptide.

A computer readable medium containing the nucleic acid sequences of the invention as well as methods of use for the computer readable medium are provided. This medium allows a nucleic acid segment corresponding to a nucleic acid sequence listed in sequence identifiers numbered 1 to 342; 473 to 539; 540 to 673, and 674 to 779 respectively, and the corresponding genes to be used as a reference sequence to search against databases. This medium allows for computer-based manipulation of a nucleic acid sequence corresponding to a nucleic acid sequence listed in sequence identifiers numbered 1 to 342; 473 to 539; 540 to 673, and 674 to 779 respectively, and the corresponding gene and polypeptide encoded by the nucleic acid sequence. Preferably the nucleic acid sequences of the invention encode polypeptides involved with silencing-related RNA and DNA metabolism (e.g., RNA helicases, RNAses, reverse transcriptase, histones, histone acetyltransferases); signal transduction (e.g., protein kinases, receptors, and calmodulin); transcription factors; stress-related and pathogen-related proteins; and general metabolism. More prefered are reverse transcribase-like protein and histone acetyltransferase-like protein.

Definitions

Certain terms used in describing the present invention are defined in the following section

“Altered levels” refers to the level of expression in transgenic organisms that differs from that of normal or untransformed organisms.

The term “altered plant trait” means any phenotypic or genotypic change in a transgenic plant relative to the wild-type or non-transgenic plant host.

“Antisense inhibition” refers to the production of antisense RNA transcripts capable of suppressing the expression of protein from an endogenous gene or a transgene.

The term “average expression” is used here as the average level of expression found in all lines that do express detectable amounts of reporter gene, so leaving out of the analysis plants that do not express any detectable reporter mRNA or -protein.

“Chimeric” is used to indicate that a DNA sequence, such as a vector or a gene, is comprised of more than one DNA sequences of distinct origin with are fused together by recombinant DNA techniques resulting in a DNA sequence, which does not occur naturally.

The term “chimeric gene” refers to any gene that contains 1) DNA sequences, including regulatory and coding sequences, that are not found together in nature, or 2) sequences encoding parts of proteins not naturally adjoined, or 3) parts of promoters that are not naturally adjoined. Accordingly, a chimeric gene may comprise regulatory sequences and coding sequences that are derived from different sources, or comprise regulatory sequences and coding sequences derived from the same source, but arranged in a manner different from that found in nature.

“Chromosomally-integrated” refers to the integration of a foreign gene or DNA construct into the host DNA by covalent bonds. Where genes are not “chromosomally integrated” they may be “transiently expressed.” Transient expression of a gene refers to the expression of a gene that is not integrated into the host chromosome but functions independently, either as part of an autonomously replicating plasmid or expression cassette, for example, or as part of another biological system such as a virus.

The terms “cis-acting sequence” and “cis-acting element” refer to DNA or RNA sequences whose functions require them to be on the same molecule. An example of a cis-acting sequence on the replicon is the viral replication origin.

“Cloning vectors” typically contain one or a small number of restriction endonuclease recognition sites at which foreign DNA sequences can be inserted in a determinable fashion without loss of essential biological function of the vector, as well as a marker gene that is suitable for use in the identification and selection of cells transformed with the cloning vector. Marker genes typically include genes that provide tetracycline resistance, hygromycin resistance or ampicillin resistance.

“Coding sequence” refers to a DNA or RNA sequence that codes for a specific amino acid sequence and excludes the non-coding sequences. It may constitute an “uninterrupted coding sequence”, i.e., lacking an intron, such as in a cDNA or it may include one or more introns bounded by appropriate splice junctions. An “intron” is a sequence of RNA which is contained in the primary transcript but which is removed through cleavage and re-ligation of the RNA within the cell to create the mature mRNA that can be translated into a protein.

“Constitutive expression” refers to expression using a constitutive or regulated promoter. “Conditional” and “regulated expression” refer to expression controlled by a regulated promoter.

“Constitutive promoter” refers to a promoter that is able to express the gene that it controls in all or nearly all of the plant tissues during all or nearly all developmental stages of the plant. Each of the transcription-activating elements do not exhibit an absolute tissue-specificity, but mediate transcriptional activation in most plant parts at a level of ≧1% of the level reached in the part of the plant in which transcription is most active.

The term “contacting” may include any method known or described for introducing a nucleic acid into a cell.

“Episome” and “replicon” refer to a DNA or RNA virus or a vector that undergoes episomal replication in plant cells. It contains cis-acting viral sequences, such as the replication origin, necessary for replication. It may or may not contain trans-acting viral sequences necessary for replication, such as the viral replication genes (for example, the AC1 and AL1 genes in ACMV and TGMV geminiviruses, respectively). It may or may not contain a target gene for expression in the host plant.

“Expression” refers to the transcription and/or translation of an endogenous gene or a transgene in plants. For example, in the case of antisense constructs, expression may refer to the transcription of the antisense DNA only. In addition, expression refers to the transcription and stable accumulation of sense (mRNA) or functional RNA. Expression may also refer to the production of protein.

“Expression cassette” as used herein means a DNA sequence capable of directing expression of a particular nucleotide sequence in an appropriate host cell, comprising a promoter operably linked to the nucleotide sequence of interest which is operably linked to termination signals. It also typically comprises sequences required for proper translation of the nucleotide sequence. The coding region usually codes for a protein of interest but may also code for a functional RNA of interest, for example antisense RNA, an untranslated RNA, a transfer RNA or a small nuclear RNA in the sense or antisense direction. The expression cassette comprising the nucleotide sequence of interest may be chimeric, meaning that at least one of its components is heterologous with respect to at least one of its other components. The expression cassette may also be one which is naturally occurring but has been obtained in a recombinant form useful for heterologous expression. The expression of the nucleotide sequence in the expression cassette may be under the control of a constitutive promoter or of an inducible promoter which initiates transcription only when the host cell is exposed to some particular external stimulus. In the case of a multicellular organism, the promoter can also be specific to a particular tissue or organ or stage of development.

The “expression pattern” of a promoter (with or without enhancer) is the pattern of expression levels which shows where in the plant and in what developmental stage transcription is initiated by the promoter. Expression patterns of a set of promoters are said to be complementary when the expression pattern of one promoter shows little overlap with the expression pattern of the other promoter. The level of expression of a promoter can be determined by measuring the ‘steady state’ concentration of a standard transcribed reporter mRNA. This measurement is indirect since the concentration of the reporter mRNA is dependent not only on its synthesis rate, but also on the rate with which the mRNA is degraded. Therefore the steady state level is the product of synthesis rates and degradation rates.

The rate of degradation can however be considered to proceed at a fixed rate when the transcribed sequences are identical, and thus this value can serve as a measure of synthesis rates. When promoters are compared in this way techniques available to those skilled in the art are hybridization S1-RNAse analysis. Northern blots and competitive RT-PCR. This list of techniques in no way represents all available techniques, but rather describes commonly used procedures used to analyze transcription activity and expression levels of mRNA.

The analysis of transcription start points in practically all promoters has revealed that there is usually no single base at which transcription starts, but rather a more or less clustered set of initiation sites, each of which accounts for some start points of the mRNA. Since this distribution varies from promoter to promoter the sequences of the reporter mRNA in each of the populations would differ from each other. Since each mRNA species is more or less prone to degradation, no single degradation rate can be expected for different reporter mRNAs. It has been shown for various eukaryotic promoter sequences that the sequence surrounding the initiation site (‘initiator’) plays an important role in determining the level of RNA expression directed by that specific promoter. This includes also part of the transcribed sequences. The direct fusion of promoter to reporter sequences would therefore lead to suboptimal levels of transcription.

A commonly used procedure to analyze expression patterns and levels is through determination of the ‘steady state’ level of protein accumulation in a cell. Commonly used candidates for the reporter gene, known to those skilled in the art are 9-glucuronidase (GUS), Chloramphenicol Acetyl Transferase (CAT) and proteins with fluorescent properties, such as Green Fluorescent Protein (GFP) from Aequora victoria. In principle, however, many more proteins are suitable for this purpose, provided the protein does not interfere with essential plant functions. For quantification and determination of localization a number of tools are suited. Detection systems can readily be created or are available which are based on e.g. immunochemical, enzymatic, fluorescent detection and quantification. Protein levels can be determined in plant tissue extracts or in intact tissue using in situ analysis of protein expression.

Generally, individual transformed lines with one chimeric promoter reporter construct will vary in their levels of expression of the reporter gene. Also frequently observed is the phenomenon that such transformants do not express any detectable product (RNA or protein). The variability in expression is commonly ascribed to ‘position effects, although the molecular mechanisms underlying this inactivity are usually not clear.

“5′ non-coding sequence” refers to a nucleotide sequence located 5′ (upstream) to the coding sequence. It is present in the fully processed mRNA upstream of the initiation codon and may affect processing of the primary transcript to mRNA, mRNA stability or translation efficiency. Turner et al., Molecular Biotechnology, 3:225 (1995).

A “functional RNA” refers to an antisense RNA, ribozyme, transfer RNA, small nuclear RNA, or other RNA that is not translated.

The term “gene” is used broadly to refer to any segment of nucleic acid associated with a biological function. Thus, genes include coding sequences and/or the regulatory sequences required for their expression. For example, gene refers to a nucleic acid fragment that expresses mRNA, functional RNA, or specific protein, including regulatory sequences. Genes also include nonexpressed DNA segments that, for example, form recognition sequences for other proteins. Genes can be obtained from a variety of sources, including cloning from a source of interest or synthesizing from known or predicted sequence information, and may include sequences designed to have desired parameters.

“Genetically stable” and “heritable” refer to chromosomally-integrated genetic elements that are stably maintained in the plant and stably inherited by progeny through successive generations.

“Genome” refers to the complete genetic material of an organism.

“Germline cells” refer to cells that are destined to be gametes and whose genetic material is heritable.

The terms “heterologous DNA sequence,” “exogenous DNA segment” or “heterologous polynucleic acid,” as used herein, each refer to a sequence that originates from a source foreign to the particular host cell or, if from the same source, is modified from its original form. Thus, a heterologous gene in a host cell includes a gene that is endogenous to the particular host cell but has been modified through, for example, the use of DNA shuffling. The terms also include non-naturally occurring multiple copies of a naturally occurring DNA sequence. Thus, the terms refer to a DNA segment that is foreign or heterologous to the cell, or homologous to the cell but in a position within the host cell nucleic acid in which the element is not ordinarily found. Exogenous DNA segments are expressed to yield exogenous polypeptides.

A “homologous” DNA sequence is a DNA sequence that is naturally associated with a host cell into which it is introduced.

“Homologous to” refers to the similarity between the nucleotide sequence of two nucleic acid molecules or between the amino acid sequences of two protein molecules. Estimates of such homology are provided by either DNA-DNA or DNA-RNA hybridization under conditions of stringency as is well understood by those skilled in the art (as described in Haines and Higgins (eds.), Nucleic Acid Hybridization, IRL Press, Oxford, U.K.), or by the comparison of sequence similarity between two nucleic acids or proteins.

Hybridization of polynucleic acid sequences may be carried out under stringent conditions. “Stringent hybridization conditions” and “stringent hybridization wash conditions” in the context of nucleic acid hybridization experiments such as Southern and Northern hybridization are sequence dependent, and are different under different environmental parameters. Longer sequences hybridize specifically at higher temperatures. An extensive guide to the hybridization o nucleic acids is found in Tijssen, Laboratory Techniques in Biochemistry and Molecular biology-Hybridization with Nucleic Acid Probes, page 1, chapter 2 “Overview of principles of hybridization and the strategy of nucleic acid probe assays” Elsevier, N.Y. (1993). Generally, highly stringent hybridization and wash conditions are selected to be about 5/C. lower than the thermal melting point (Tₘ) for the specific sequence at a defined ionic strength and pH. Typically, under “stringent conditions” a probe will hybridize to its target subsequence, but to no other sequences. For example, by “stringent conditions” or “stringent hybridization conditions” is intended conditions under which a probe will hybridize to its target sequence to a detectably greater degree than to other sequences (e.g., at least 2- fold over background). By controlling the stringency of the hybridization and/or washing conditions, target sequences that are 100% complementary to the probe can be identified (homologous probing). Alternatively, stringency conditions can be adjusted to allow some mismatching in sequences so that lower degrees of similarity are detected (heterologous probing). Generally, a probe is less than about 1000 nucleotides in length, preferably less than 500 nucleotides in length.

Typically, stringent conditions will be those in which the salt concentration is less than about 1.5 M Na ion, typically about 0.01 to 1.0 M Na ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide.

Exemplary low stringency conditions include hybridization with a buffer solution of 30 to 35% formamide, 1 M NaCl, 1% SDS (sodium dodecyl sulphate) at 37° C., and a wash in 1× to 2×SSC (20×SSC=3.0 M NaCl/0.3 M trisodium citrate) at 50 to 55° C. Exemplary moderate stringency conditions include hybridization in 40 to 45% formamide, 1.0 M NaCl, 1% SDS at 37° C., and a wash in 0.5× to 1×SSC at 55 to 60° C. Exemplary high stringency conditions include hybridization in 50% formamide, 1 M NaCl, 1% SDS at 37° C., and a wash in 0.1×SSC at 60 to 65° C.

Specificity is typically the function of post-hybridization washes, the critical factors being the ionic strength and temperature of the final wash solution. For DNA-DNA hybrids, the Tₘ can be approximated from the equation of Meinkoth and Wahl Anal. Biochem. 138:267-284 (1984); Tₘ 81.5° C.+16.6 (log M)+0.41 (%GC)−0.61 (% form)−500/L; where M is the molarity of monovalent cations, %GC is the percentage of guanosine and cytosine nucleotides in the DNA, % form is the percentage of formamide in the hybridization solution, and L is the length of the hybrid in base pairs. The Tₘ is the temperature (under defined ionic strength and pH) at which 50% of a complementary target sequence hybridizes to a perfectly matched probe.

Very stringent conditions are selected to be equal to the Tₘ for a particular probe. An example of stringent hybridization conditions for hybridization of complementary nucleic acids which have more than 100 complementary residues on a filter in a Southern or Northern blot is 50% formamide with 1 mg of heparin at 42/C., with the hybridization being carried out overnight. An example of highly stringent conditions is 0.15 M NaCl at 72/C. for about 15 minutes. An example of stringent wash conditions is a 0.2×SSC wash at 65/C. for 15 minutes (see, Sambrook, infra, for a description of SSC buffer). Often, a high stringency wash is preceded by a low stringency wash to remove background probe signal. An example of medium stringency for a duplex of, e.g., more than 100 nucleotides, is 1×SSC at 45/C. for 15 minutes. An example low stringency wash for a duplex of, e.g., more than 100 nucleotides, is 4-6×SSC at 40/C. for 15 minutes. For short probes (e.g., about 10 to 50 nucleotides), stringent conditions typically involve salt concentrations of less than about 1.0M Na ion, typically about 0.01 to 1.0 M Na ion concentration (or other slats) at pH 7.0 to 8.3, and the temperature is typically at least about 30/C. Stringent conditions can also be achieved with the addition of destabilizing agents such as formamide. In general, a signal to noise ratio of 2× (or higher) than that observed for an unrelated probe in the particular hybridization assay indicates detection of a specific hybridization. Nucleic acids that do not hybridize to each other under stringent conditions are still substantially identical if the proteins that they encode are substantially identical. This occurs, e.g., when a copy of a nucleic acid is created using the maximum codon degeneracy permitted by the genetic code.

The following are examples of sets of hybridization/wash conditions that may be used to clone homologous polynucleic acid sequences that are substantially identical to the polynucleic acid segments of the present invention: the homologous polynucleic acid sequence preferably hybridizes to the polynucleic acid segment in 7% sodium dodecyl sulfate (SDS), 0.5 M NaPO₄, 1 mM EDTA at 50/C. with washing in 2×SSC, 0.1% SDS at 50/C., more desirably in 7% sodium dodecyl sulfate (SDS), 0.5 M NaPO₄, 1 mM EDTA at 50/C. with washing in 1×SSC, 0.1% SDS at 50/C., more desirably still in 7% sodium dodecyl sulfate (SDS), 0.5 M NaPO₄, 1 mM EDTA at 50/C. with washing in 0.5×SSC, 0.1% SDS at 50/C., preferably in 7% sodium dodecyl sulfate (SDS), 0.5 M NaPO₄, 1 mM EDTA at 50/C. with washing in 0.1×SSC, 0.1% SDS at 50/C., more preferably in 7% sodium dodecyl sulfate (SDS), 0.5 M NaPO₄, 1 mM EDTA at 50/C. with washing in 0.1×SSC, 0.1% SDS at 65/C.

Tₘ is reduced by about 1° C. for each 1% of mismatching; thus, Tₘ, hybridization, and/or wash conditions can be adjusted to hybridize to sequences of the desired identity. For example, if polynucleic acid sequences with >90% identity are sought, the Tₘ can be decreased 10° C. Generally, stringent conditions are selected to be about 5° C. lower than the thermal melting point (Tₘ) for the specific sequence and its complement at a defined ionic strength and pH. However, severely stringent conditions can utilize a hybridization and/or wash at 1, 2, 3, or 4° C. lower than the thermal melting point (Tₘ); moderately stringent conditions can utilize a hybridization and/or wash at 6, 7, 8, 9, or 10° C. lower than the thermal melting point (Tₘ); low stringency conditions can utilize a hybridization and/or wash at 11, 12, 13, 14, 15, or 20° C. lower than the thermal melting point (Tₘ). Using the equation, hybridization and wash compositions, and desired T, those of ordinary skill will understand that variations in the stringency of hybridization and/or wash solutions are inherently described. If the desired degree of mismatching results in a T of less than 45° C. (aqueous solution) or 32° C. (formamide solution), it is preferred to increase the SSC concentration so that a higher temperature can be used. An extensive guide to the hybridization of nucleic acids is found in Tijssen (1993) Laboratory Techniques in Biochemistry and Molecular Biology-Hybridization with Nucleic Acid Probes, Part 1, Chapter 2 (Elsevier, N.Y.); and Ausubel et al., eds. (1995) Current Protocols in Molecular Biology, Chapter 2 (Greene Publishing and Wiley—Interscience, New York). See Sambrook et al. (1989) Molecular Cloning: A Laboratory Manual (2d ed., Cold Spring Harbor Laboratory Press, Plainview, N.Y.).

Generally, stringent conditions are selected to be about 5° C. lower than the thermal melting point (Tm) for the specific sequence at a defined ionic strength and pH. However, stringent conditions encompass temperatures in the range of about 1° C. to about 20° C., depending upon the desired degree of stringency as otherwise qualified herein. Polynucleic acid sequences that do not hybridize to each other under stringent conditions are still substantially identical if the polypeptides they encode are substantially identical. This may occur, e.g., when a copy of a nucleic acid is created using the maximum codon degeneracy permitted by the genetic code. One indication that two polynucleic acid sequences are substantially identical is when the polypeptide encoded by the first nucleic acid is immunologically cross reactive with the polypeptide encoded by the second nucleic acid.

The terms “in cis” and “in trans” refer to the presence of DNA elements, such as the viral origin of replication and the replication protein(s) gene, on the same DNA molecule or on a different DNA molecule, respectively.

“Inducible promoter” refers to those regulated promoters that can be turned on in one or more cell types by an external stimulus, such as a chemical, light, hormone, stress, or a pathogen.

The “initiation site” is the position surrounding the first nucleotide that is part of the transcribed sequence, which is also defined as position +1. With respect to this site all other sequences of the gene and its controlling regions are numbered. Downstream sequences (i.e. further protein encoding sequences in the 3′ direction) are denominated positive, while upstream sequences (mostly of the controlling regions in the 5′ direction) are denominated negative.

The term “intracellular localization sequence” refers to a nucleotide sequence that encodes an intracellular targeting signal. An “intracellular targeting signal” is an amino acid sequence that is translated in conjunction with a protein and directs it to a particular sub-cellular compartment. “Endoplasmic reticulum (ER) stop transit signal” refers to a carboxy-terminal extension of a polypeptide, which is translated in conjunction with the polypeptide and causes a protein that enters the secretory pathway to be retained in the ER. “ER stop transit sequence” refers to a nucleotide sequence that encodes the ER targeting signal. Other intracellular targeting sequences encode targeting signals active in seeds and/or leaves and vacuolar targeting signals.

The invention encompasses isolated or substantially purified nucleic acid or protein compositions. In the context of the present invention, an “isolated” or “purified” polynucleic acid segment or an “isolated” or “purified” polypeptide is a polynucleic acid segment or polypeptide that, by the hand of man, exists apart from its native environment and is therefore not a product of nature. An isolated polynucleic acid segment or polypeptide may exist in a purified form or may exist in a non-native environment such as, for example, a transgenic host cell. For example, an “isolated” or “purified” polynucleic acid segment or protein, or biologically active portion thereof, is substantially free of other cellular material, or culture medium when produced by recombinant techniques, or substantially free of chemical precursors or other chemicals when chemically synthesized.

Preferably, an “isolated” polynucleic acid is free of sequences (preferably protein encoding sequences) that naturally flank the nucleic acid (i.e., sequences located at the 5′ and 3′ ends of the nucleic acid) in the genomic DNA of the organism from which the nucleic acid is derived. For example, in various embodiments, the isolated nucleic acid molecule can contain less than about 5 kb, 4 kb, 3 kb, 2 kb, 1 kb, 0.5 kb, or 0.1 kb of nucleotide sequences that naturally flank the nucleic acid molecule in genomic DNA of the cell from which the nucleic acid is derived. A protein that is substantially free of cellular material includes preparations of protein or polypeptide having less than about 30%, 20%, 10%, 5%, (by dry weight) of contaminating protein. When the protein of the invention, or biologically active portion thereof, is recombinantly produced, preferably culture medium represents less than about 30%, 20%, 10%, or 5% (by dry weight) of chemical precursors or non-protein-of-interest chemicals.

Fragments and variants of the disclosed nucleotide sequences and proteins or partial-length proteins encoded thereby are also encompassed by the present invention. By “fragment” is intended a portion of the nucleotide sequence or a portion of the amino acid sequence, and hence a portion of the polypeptide or protein, encoded thereby. Alternatively, fragments of a polynucleic acid sequence that are useful as hybridization probes generally do not encode fragment proteins retaining biological activity. Thus, fragments of a nucleotide sequence may range from at least about 9 nucleotides, about 12 nucleotides, about 20 nucleotides, about 50 nucleotides, about 100 nucleotides or more.

A “marker gene” encodes a selectable or screenable trait.

The term “mature” protein refers to a post-translationally processed polypeptide without its signal peptide. “Precursor” protein refers to the primary product of translation of an mRNA. “Signal peptide” refers to the amino terminal extension of a polypeptide, which is translated in conjunction with the polypeptide forming a precursor peptide and which is required for its entrance into the secretory pathway. The term “signal sequence” refers to a nucleotide sequence that encodes the signal peptide.

The terms “modulate”, “modulates” and are used to broadly refer to alteration of the quantity or activity of a product that is expressed within a cell. As used herein, the alteration may include increased or decreased transcription or translation of a gene.

The term “native gene” refers to gene that is present in the genome of an untransformed cell.

“Naturally occurring” is used to describe an object that can be found in nature as distinct from being artificially produced by man. For example, a protein or nucleotide sequence present in an organism (including a virus), which can be isolated from a source in nature and which has not been intentionally modified by man in the laboratory, is naturally occurring.

“Non-specific expression” refers to constitutive expression or low level, basal (‘leaky’) expression in nondesired cells or tissues from a ‘regulated promoter’.

The term “nucleic acid”, “polynucleic acid” or “polynucleic acid segment” refers to deoxyribonucleotides or ribonucleotides and polymers thereof in either single- or double-stranded form, composed of monomers (nucleotides) containing a sugar, phosphate and a base which is either a purine or pyrimidine. Unless specifically limited, the term encompasses nucleic acids containing known analogs of natural nucleotides which have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions) and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res., 19:5081 (1991); Ohtsuka et al., J. Biol. Chem., 260:2605 (1985); Rossolini et al., Mol. Cell. Probes, 8:91 (1994)).

A “nucleic acid fragment” is a fraction of a given nucleic acid molecule. In higher plants, deoxyribonucleic acid (DNA) is the genetic material while ribonucleic acid (RNA) is involved in the transfer of information contained within DNA into proteins. A “genome” is the entire body of genetic material contained in each cell of an organism. The term “nucleotide sequence” refers to a polymer of DNA or RNA which can be single- or double-stranded, optionally containing synthetic, non-natural or altered nucleotide bases capable of incorporation into DNA or RNA polymers. The terms “nucleic acid” or “nucleic acid sequence” may also be used interchangeably with gene, cDNA, DNA and RNA encoded by a gene.

An “oligonucleotide” for use in probing or amplification reactions may be about 30 or fewer nucleotides in length (e.g., 9, 12, 15, 18, 20, 21 or 24, or any number between 9 and 30). Generally specific primers are upwards of 14 nucleotides in length. For optimum specificity and cost effectiveness, primers of 16-24 nucleotides in length may be preferred. Those skilled in the art are well versed in the design of primers for use processes such as PCR. If required, probing can be done with entire restriction fragments of the gene disclosed herein which may be 100's or even 1000's of nucleotides in length.

The terms “open reading frame” and “ORF” refer to the amino acid sequence encoded between translation initiation and termination codons of a coding sequence. The terms “initiation codon” and “termination codon” refer to a unit of three adjacent nucleotides (‘codon’) in a coding sequence that specifies initiation and chain termination, respectively, of protein synthesis (mRNA translation).

“Operably linked” means joined as part of the same nucleic acid molecule, suitably positioned and oriented for transcription to be initiated from the promoter. DNA operably linked to a promoter is “under transcriptional initiation regulation” of the promoter. Coding sequences can be operably-linked to regulatory sequences in sense or antisense orientation.

“Overexpression” refers to the level of expression in transgenic organisms that exceeds levels of expression in normal or untransformed organisms.

Known methods of polymerase chain reaction “PCR” include, but are not limited to, methods using paired primers, nested primers, single specific primers, degenerate primers, gene-specific primers, vector-specific primers, partially mismatched primers, and the like. See also Innis et al., eds., PCR Protocols: A Guide to Methods and Applications (Academic Press, New York (1995); and Gelfand, eds., PCR Strategies (Academic Press, New York (1995); and Innis and Gelfand, eds., PCR Methods Manual (Academic Press, New York) (1999).

“Plant tissue” includes differentiated and undifferentiated tissues or plants, including but not limited to roots, stems, shoots, leaves, pollen, seeds, tumor tissue and various forms of cells and culture such as single cells, protoplast, embryos, and callus tissue. The plant tissue may be in plants or in organ, tissue or cell culture.

“Primary transformant” and “TO generation” refer to transgenic plants that are of the same genetic generation as the tissue which was initially transformed (i.e., not having gone through meiosis and fertilization since transformation). “Production tissue” refers to mature, harvestable tissue consisting of non-dividing, terminally-differentiated cells. It excludes young, growing tissue consisting of germline, meristematic, and not-fully-differentiated cells.

“Promoter” refers to a nucleotide sequence, usually upstream (5′) to its coding sequence, which controls the expression of the coding sequence by providing the recognition for RNA polymerase and other factors required for proper transcription. “Promoter” includes a minimal promoter that is a short DNA sequence comprised of a TATA-box and other sequences that serve to specify the site of transcription initiation, to which regulatory elements are added for control of expression. “Promoter” also refers to a nucleotide sequence that includes a minimal promoter plus regulatory elements that is capable of controlling the expression of a coding sequence or functional RNA. This type of promoter sequence consists of proximal and more distal upstream elements, the latter elements often referred to as enhancers. Accordingly, an “enhancer” is a DNA sequence which can stimulate promoter activity and may be an innate element of the promoter or a heterologous element inserted to enhance the level or tissue specificity of a promoter. It is capable of operating in both orientations (normal or flipped), and is capable of functioning even when moved either upstream or downstream from the promoter. Both enhancers and other upstream promoter elements bind sequence-specific DNA-binding proteins that mediate their effects. Promoters may be derived in their entirety from a native gene, or be composed of different elements derived from different promoters found in nature, or even be comprised of synthetic DNA segments. A promoter may also contain DNA sequences that are involved in the binding of protein factors which control the effectiveness of transcription initiation in response to physiological or developmental conditions.

Promoter elements, particularly a TATA element, that are inactive or that have greatly reduced promoter activity in the absence of upstream activation are referred to as “minimal or core promoters.” In the presence of a suitable transcription factor, the minimal promoter functions to permit transcription. A “minimal or core promoter” thus consists only of all basal elements needed for transcription initiation, e.g., a TATA box and/or an initiator.

The terms “protein,” “peptide” and “polypeptide” are used interchangeably herein.

“Regulated promoter” refers to promoters that direct gene expression not constitutively, but in a temporally- and/or spatially-regulated manner, and include both tissue-specific and inducible promoters. It includes natural and synthetic sequences as well as sequences which may be a combination of synthetic and natural sequences. Different promoters may direct the expression of a gene in different tissues or cell types, or at different stages of development, or in response to different environmental conditions. New promoters of various types useful in plant cells are constantly being discovered, numerous examples may be found in the compilation by Okamuro et al., Biochemistry of Plants, 15:1 (1989). Since in most cases the exact boundaries of regulatory sequences have not been completely defined, DNA fragments of different lengths may have identical promoter activity. Typical regulated promoters useful in plants include but are not limited to safener-inducible promoters, promoters derived from the tetracycline-inducible system, promoters derived from salicylate-inducible systems, promoters derived from alcohol-inducible systems, promoters derived from glucocorticoid-inducible system, promoters derived from pathogen-inducible systems, and promoters derived from ecdysome-inducible systems.

“Regulatory sequences” and “suitable regulatory sequences” each refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences include enhancers, promoters, translation leader sequences, introns, and polyadenylation signal sequences. They include natural and synthetic sequences as well as sequences which may be a combination of synthetic and natural sequences. As is noted above, the term “suitable regulatory sequences” is not limited to promoters. However, some suitable regulatory sequences useful in the present invention will include, but are not limited to constitutive plant promoters, plant tissue-specific promoters. plant development-specific promoters, inducible plant promoters and viral promoters.

“Replication origin” refers to a cis-acting replication sequence essential for viral or episomal replication.

The term “RNA transcript” refers to the product resulting from RNA polymerase catalyzed transcription of a DNA sequence. When the RNA transcript is a perfect complementary copy of the DNA sequence, it is referred to as the primary transcript or it may be a RNA sequence derived from posttranscriptional processing of the primary transcript and is referred to as the mature RNA. “Messenger RNA” (mRNA) refers to the RNA that is without introns and that can be translated into protein by the cell. “cDNA” refers to a single- or a double-stranded DNA that is complementary to and derived from mRNA.

“Secondary transformants” and the “T1, T2, T3, etc. generations” refer to transgenic plants derived from primary transformants through one or more meiotic and fertilization cycles. They may be derived by self-fertilization of primary or secondary transformants or crosses of primary or secondary transformants with other transformed or untransformed plants.

The following terms are used to describe the sequence relationships between two or more nucleic acids or polynucleotides: (a) “reference sequence”, (b) “comparison window”, (c) “sequence identity”, (d) “percentage of sequence identity”, and (e) “substantial identity”.

(a) As used herein, “reference sequence” is a defined sequence used as a basis for sequence comparison. A reference sequence may be a subset or the entirety of a specified sequence; for example, as a segment of a full length cDNA or gene sequence, or the complete cDNA or gene sequence.

(b) As used herein, “comparison window” makes reference to a contiguous and specified segment of a polynucleic acid sequence, wherein the polynucleic acid sequence in the comparison window may comprise additions or deletions (i.e., gaps) compared to the reference sequence (which does not comprise additions or deletions) for optimal alignment of the two sequences. Generally, the comparison window is at least 20 contiguous nucleotides in length, and optionally can be 30, 40, 50, 100, or longer. Those of skill in the art understand that to avoid a high similarity to a reference sequence due to inclusion of gaps in the polynucleotide sequence a gap penalty is typically introduced and is subtracted from the number of matches.

Methods of alignment of sequences for comparison are well known in the art. Thus, the determination of percent identity between any two sequences can be accomplished using a mathematical algorithm. Preferred, non-limiting examples of such mathematical algorithms are the algorithm of Myers and Miller, CABIOS, 4:11 (1988); the local homology algorithm of Smith et al., Adv. Appl. Math., 2:482 (1981); the homology alignment algorithm of Needleman and Wunsch, J. Mol. Biol., 48:443 (1970); the search-for-similarity-method of Pearson and Lipman, Proc. Natl. Acad. Sci., 85:2444 (1988); the algorithm of Karlin and Altschul, Proc. Natl. Acad Sci. USA, 87:2264 (1990), modified as in Karlin and Altschul, Proc. Natl. Acad. Sci. USA, 90:5873 (1993).

Computer implementations of these mathematical algorithms can be utilized for comparison of sequences to determine sequence identity. Such implementations include, but are not limited to: CLUSTAL in the PC/Gene program (available from Intelligenetics, Mountain View, Calif.); the ALIGN program (Version 2.0) and GAP, BESTFIT, BLAST, FASTA, and TFASTA in the Wisconsin Genetics Software Package, Version 8 (available from Genetics Computer Group (GCG), 575 Science Drive, Madison, Wis., USA). Alignments using these programs can be performed using the default parameters. The CLUSTAL program is well described by Higgins et al., Gene, 73:237 (1988); Higgins et al., CABIOS, 5:151 (1989); Corpet et al., Nucleic Acids Res., 16:10881 (1988); Huang et al., CABIOS, 8:155 (1992); and Pearson et al., Meth. Mol. Biol., 24:307 (1994). The ALIGN program is based on the algorithm of Myers and Miller, supra. The BLAST programs of Altschul et al., J. Mol. Biol., 215:403 (1990), are based on the algorithm of Karlin and Altschul supra. To obtain gapped alignments for comparison purposes, Gapped BLAST (in BLAST 2.0) can be utilized as described in Altschul et al., Nucleic Acids Res., 25:3389 (1997). Alternatively, PSI-BLAST (in BLAST 2.0) can be used to perform an iterated search that detects distant relationships between molecules. See Altschul et al., supra. When utilizing BLAST, Gapped BLAST, PSI-BLAST, the default parameters of the respective programs (e.g. BLASTN for nucleotide sequences, BLASTX for proteins) can be used. The BLASTN program (for nucleotide sequences) uses as defaults a wordlength (W) of 11, an expectation (E) of 10, a cutoff of 100, M=5, N=−4, and a comparison of both strands. For amino acid sequences, the BLASTP program uses as defaults a wordlength (W) of 3, an expectation (E) of 10, and the BLOSUM62 scoring matrix (see Henikoff & Henikoff, Proc. Natl. Acad. Sci. USA 89, 10915 (1989)). See http://www.ncbi.n1m.nih.gov. Alignment may also be performed manually by inspection.

For purposes of the present invention, comparison of polynucleic acid sequences for determination of percent sequence identity to the polynucleic acid segments disclosed herein is preferably made using the BlastN program (version 1.4.7 or later) with its default parameters or any equivalent program. By “equivalent program” is intended any sequence comparison program that, for any two sequences in question, generates an alignment having identical nucleotide or amino acid residue matches and an identical percent sequence identity when compared to the corresponding alignment generated by the preferred program.

(c) As used herein, “sequence identity” or “identity” in the context of two polynucleic acid or polypeptide sequences makes reference to the residues in the two sequences that are the same when aligned for maximum correspondence over a specified comparison window. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity.” Means for making this adjustment are well known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., as implemented in the program PC/GENE (Intelligenetics, Mountain View, Calif.).

(d) As used herein, “percentage of sequence identity” means the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity.

(e)(i) The term “substantial identity” of polynucleotide sequences means that a polynucleotide comprises a sequence that has at least 70%, preferably at least 80%, more preferably at least 90%, and most preferably at least 95%, sequence identity, and single unit percentage identities based on these classes. For example 71%, 72%, 73% and the like, up through at least the 95% class as compared to a reference sequence using one of the alignment programs described using standard parameters. One of skill in the art will recognize that these values can be appropriately adjusted to determine corresponding identity of proteins encoded by two nucleotide sequences by taking into account codon degeneracy, amino acid similarity, reading frame positioning, and the like. Substantial identity of amino acid sequences for these purposes normally means sequence identity of at least 70%, more preferably at least 80%, 90%, and most preferably at least 95%.

Another indication that nucleotide sequences are substantially identical is if two molecules hybridize to each other under stringent conditions. Generally, stringent conditions are selected to be about 5° C. lower than the thermal melting point (Tₘ) for the specific sequence at a defined ionic strength and pH. However, stringent conditions encompass temperatures in the range of about 1° C. to about 20° C., depending upon the desired degree of stringency as otherwise qualified herein. Nucleic acids that do not hybridize to each other under stringent conditions are still substantially identical if the polypeptides they encode are substantially identical. This may occur, e.g., when a copy of a nucleic acid is created using the maximum codon degeneracy permitted by the genetic code. One indication that two nucleic acid sequences are substantially identical is when the polypeptide encoded by the first nucleic acid is immunologically cross reactive with the polypeptide encoded by the second nucleic acid.

(e)(ii) The term “substantial identity” in the context of a peptide indicates that a peptide comprises a sequence with at least 70%, preferably at least 80%, more preferably at least 90%, and most preferably at least 95%, sequence identity, and single unit percentage identities based on these classes. For example 71%, 72%, 73% and the like, up through at least the 95% class as compared to a reference sequence over a specified comparison window. Preferably, optimal alignment is conducted using the homology alignment algorithm of Needleman and Wunsch J. Mol. Biol., 48:443, (1970). An indication that two peptide sequences are substantially identical is that one peptide is immunologically reactive with antibodies raised against the second peptide. Thus, a peptide is substantially identical to a second peptide, for example, where the two peptides differ only by a conservative substitution.

“Specific expression” is the expression of gene products which is limited to one or a few plant tissues (spatial limitation) and/or to one or a few plant developmental stages (temporal limitation). It is acknowledged that hardly a true specificity exists: promoters seem to be preferably switch on in some tissues, while in other tissues there can be no or only little activity. This phenomenon is known as leaky expression. However, with specific expression in this invention is meant preferable expression in one or a few plant tissues.

“Stably transformed” refers to cells that have been selected and regenerated on a selection media following transformation.

The term “substantially similar” refers to nucleotide and amino acid sequences that represent equivalents of the instant inventive sequences. For example, altered nucleotide sequences which simply reflect the degeneracy of the genetic code but nonetheless encode amino acid sequences that are identical to the inventive amino acid sequences are substantially similar to the inventive sequences. In addition, amino acid sequences that are substantially similar to the instant sequences are those wherein overall amino acid identity is 95% or greater to the instant sequences. Modifications to the instant invention that result in equivalent nucleotide or amino acid sequences is well within the routine skill in the art. Moreover, the skilled artisan recognizes that equivalent nucleotide sequences encompassed by this invention can also be defined by their ability to hybridize, under stringent conditions (0.1×SSC, 0.1% SDS, 65° C.), with the nucleotide sequences that are within the literal scope of the instant claims.

“Substantially the same activity” when used in reference to a fragment of a polypeptide or polynucleotide means that the fragment has at least 50%, more preferably at least 80%, even more preferably at least 90% to 95%, and still more preferably at least 95%, including 100%, of the activity of the full-length polypeptide.

“Target gene” refers to a gene on the replicon that expresses the desired target coding sequence, functional RNA, or protein. The target gene is not essential for replicon replication. Additionally, target genes may comprise native non-viral genes inserted into a non-native organism, or chimeric genes, and will be under the control of suitable regulatory sequences. Thus, the regulatory sequences in the target gene may come from any source, including the virus. Target genes may include coding sequences that are either heterologous or homologous to the genes of a particular plant to be transformed. However, target genes do not include native viral genes. Typical target genes include, but are not limited to genes encoding a structural protein, a seed storage protein, a protein that conveys herbicide resistance, and a protein that conveys insect resistance. Proteins encoded by target genes are known as “foreign proteins”. The expression of a target gene in a plant will typically produce an altered plant trait.

“3′ non-coding sequence” refers to nucleotide sequences located 3′ (downstream) to a coding sequence and include polyadenylation signal sequences and other sequences encoding regulatory signals capable of affecting mRNA processing or gene expression. The polyadenylation signal is usually characterized by affecting the addition of polyadenylic acid tracts to the 3′ end of the mRNA precursor. The use of different 3′ non-coding sequences is exemplified by Ingelbrecht et al., Plant Cell, 1:671 (1989).

“Tissue-specific promoter” refers to regulated promoters that are not expressed in all plant cells but only in one or more cell types in specific organs (such as leaves or seeds), specific tissues (such as embryo or cotyledon), or specific cell types (such as leaf parenchyma or seed storage cells). These also include promoters that are temporally regulated, such as in early or late embryogenesis, during fruit ripening in developing seeds or fruit, in fully differentiated leaf, or at the onset of senescence.

The terms “trans-acting sequence” and “trans-acting element” refer to DNA or RNA sequences whose function does not require them to be on the same molecule. Examples of trans-acting sequence is the replication gene (ACI or AL1 in ACMV or TGMV geminiviruses, respectively), that can function in replication without being on the replicon.

“Transactivating gene” refers to a gene encoding a transactivating protein. It can encode a viral replication protein(s) or a site-specific replicase. It can be a natural gene, for example, a viral replication gene, or a chimeric gene, for example, when plant regulatory sequences are operably-linked to the open reading frame of a site-specific recombinase or a viral replication protein. “Transactivating genes” may be chromosomally integrated or transiently expressed.

“Transcription Stop Fragment” refers to nucleotide sequences that contain one or more regulatory signals, such as polyadenylation signal sequences, capable of terminating transcription. Examples include the 3′ non-regulatory regions of genes encoding nopaline synthase and the small subunit of ribulose bisphosphate carboxylase.

The term “transformation” refers to the transfer of a nucleic acid fragment into the genome of a host cell, resulting in genetically stable inheritance. Host cells containing the transformed nucleic acid fragments are referred to as “transgenic” cells, and organisms comprising transgenic cells are referred to as “transgenic organisms”. Examples of methods of transformation of plants and plant cells include Agrobacterium-mediated transformation (De Blaere et al., Meth. Enzymol., 143:277 (1987)) and particle bombardment technology (Klein et al., (1987) Nature (London), 327:70 (1987); U.S. Pat. No. 4,945,050). Whole plants may be regenerated from transgenic cells by methods well known to the skilled artisan (see, for example, Fromm et al., Bio/Technology, 8:833 (1990)).

“Transformed,” “transgenic,” and “recombinant” refer to a host organism such as a bacterium or a plant into which a heterologous nucleic acid molecule has been introduced. The nucleic acid molecule can be stably integrated into the genome by methods generally known in the art which are disclosed in Sambrook et al., Molecular Cloning: A Laboratory Manual (2d ed., Cold Spring Harbor Laboratory Press, Plainview, N.Y.) (1989). For example, “transformed,” “transformant,” and “transgenic” plants or calli have been through the transformation process and contain a foreign gene integrated into their chromosome. The term “untransformed” refers to normal plants that have not been through the transformation process.

A “transgene” refers to a gene that has been introduced into the genome by transformation and is stably maintained. Transgenes may include, for example, genes that are either heterologous or homologous to the genes of a particular plant to be transformed. Additionally, transgenes may comprise native genes inserted into a non-native organism, or chimeric genes. The term “endogenous gene” refers to a native gene in its natural location in the genome of an organism. A “foreign” gene refers to a gene not normally found in the host organism but that is introduced by gene transfer.

“Transgene activation system” refers to the expression system comprised of an inactive transgene and a chimeric site-specific recombinase gene, functioning together, to effect transgene expression in a regulated manner. The specificity of the recombination will be determined by the specificity of regulated promoters as well as the use of wild-type or mutant site-specific sequences. Both elements of the system can be chromosomally integrated and inherited independently. Such site specific sequences are well known in the art, see for example the Cre-Lox system (U.S. Pat. No. 4,959,317) as well as the FLP/FRT site-specific recombination system. Lyznik et al., Nucleic Acids Res., 21:969 (1993).

A “transgenic plant” is a plant having one or more plant cells that contain a heterologous DNA sequence.

“Transient expression” refers to expression in cells in which a virus or a transgene is introduced by viral infection or by such methods as Agrobacterium-mediated transformation, electroporation, or biolistic bombardment, but not selected for its stable maintenance.

“Transiently transformed” refers to cells in which transgenes and foreign DNA have been introduced (for example, by such methods as Agrobacterium-mediated transformation or biolistic bombardment), but not selected for stable maintenance.

The term “translation leader sequence” refers to that DNA sequence portion of a gene between the promoter and coding sequence that is transcribed into RNA and is present in the fully processed mRNA upstream (5′) of the translation start codon. The translation leader sequence may affect processing of the primary transcript to mRNA, mRNA stability or translation efficiency.

“Translation Stop Fragment” refers to nucleotide sequences that contain one or more regulatory signals, such as one or more termination codons in all three frames, capable of terminating translation. Insertion of a translation stop fragment adjacent to or near the initiation codon at the 5′ end of the coding sequence will result in no translation or improper translation. Excision of the translation stop fragment by site-specific recombination will leave a site-specific sequence in the coding sequence that does not interfere with proper translation using the initiation codon.

By “variant” polypeptide is intended a polypeptide derived from the native protein by deletion (so-called truncation) or addition of one or more amino acids to the N-terminal and/or C-terminal end of the native protein; deletion or addition of one or more amino acids at one or more sites in the native protein; or substitution of one or more amino acids at one or more sites in the native protein. Such variants may results form, for example, genetic polymorphism or from human manipulation. Methods for such manipulations are generally known in the art.

Thus, the polypeptides of the invention may be altered in various ways including amino acid substitutions, deletions, truncations, and insertions. Methods for such manipulations are generally known in the art. For example, amino acid sequence variants of the polypeptides can be prepared by mutations in the DNA. Methods for mutagenesis and nucleotide sequence alterations are well known in the art. See, for example, Kunkel, Proc. Natl. Acad. Sci. USA, 82, 488 (1985); Kunkel et al., Methods in Enzymol., 154:367 (1987); U. S. Pat. No. 4,873,192; Walker and Gaastra, eds., Techniques in Molecular biology, MacMillan Publishing Company, New York (1983) and the references cited therein. Guidance as to appropriate amino acid substitutions that do not affect biological activity of the protein of interest may be found in the model of Dayhoff et al., Atlas of Protein Sequence and Structure, Natl. Biomed. Res. Found., Washington, C. D. (1978), herein incorporated by reference. Conservative substitutions, such as exchanging one amino acid with another having similar properties, are preferred.

Thus, the genes and nucleotide sequences of the invention include both the naturally occurring sequences as well as mutant forms. Likewise, the polypeptides of the invention encompass both naturally occurring proteins as well as variations and modified forms thereof. Such variants will continue to possess the desired activity. The deletions, insertions, and substitutions of the polypeptide sequence encompassed herein are not expected to produce radical changes in the characteristics of the polypeptide. However, when it is difficult to predict the exact effect of the substitution, deletion, or insertion in advance of doing so, one skilled in the art will appreciate that the effect will be evaluated by routine screening assays.

The nucleic acid molecules of the invention can be optimized for enhanced expression in plants of interest. See, for example, EPA035472; WO91/16432; Perlak et al., Proc. Natl. Acad. Sci. USA, 88:3324 (1991); and Murray et al., Nucleic Acids Res., 17:477 (1989). In this manner, the genes or gene fragments can be synthesized utilizing plant-preferred codons. See, for example, Campbell and Gowri, Plant Physiol., 92:1 (1990) for a discussion of host-preferred codon usage. Thus, the nucleotide sequences can be optimized for expression in any plant. It is recognized that all or any part of the gene sequence may be optimized or synthetic. That is, synthetic or partially optimized sequences may also be used. Variant nucleotide sequences and proteins also encompass sequences and protein derived from a mutagenic and recombinogenic procedure such as DNA shuffling. With such a procedure, one or more different coding sequences can be manipulated to create a new polypeptide possessing the desired properties. In this manner, libraries of recombinant polynucleotides are generated from a population of related sequence polynucleotides comprising sequence regions that have substantial sequence identity and can be homologously recombined in vitro or in vivo. Strategies for such DNA shuffling are known in the art. See, for example, Stemmer, Proc. Natl. Acad. Sci. USA, 91:10747 (1994); Stemmer, Nature, 370:389 (1994); Crameri et al., Nature Biotech., 15:436 (1997); Moore et al., J. Mol. Biol., 272:336 (1997); Zhang et al., Proc. Natl. Acad. Sci. USA, 94:4504 (1997); Crameri et al., Nature, 391:288 (1998); and U.S. Pat. Nos. 5,605,793 and 5,837,458.

By “variants” is intended substantially similar sequences. For nucleotide sequences, variants include those sequences that, because of the degeneracy of the genetic code, encode the identical amino acid sequence of the native protein. Naturally occurring allelic variants such as these can be identified with the use of well-known molecular biology techniques, as, for example, with polymerase chain reaction (PCR) and hybridization techniques. Variant nucleotide sequences also include synthetically derived nucleotide sequences, such as those generated, for example, by using site-directed mutagenesis which encode the native protein, as well as those that encode a polypeptide having amino acid substitutions. Generally, nucleotide sequence variants of the invention will have at least 40%, 50%, 60%, preferably 70%, more preferably 80%, even more preferably 90%, most preferably 99%, and single unit percentage identity to the native nucleotide sequence based on these classes. For example, 71%, 72%, 73% and the like, up to at least the 90% class. Variants may also include a full length gene corresponding to an identified gene fragment.

“Vector” is defined to include, inter alia, any plasmid, cosmid, phage or Agrobacterium binary vector in double or single stranded linear or circular form which may or may not be self transmissible or mobilizable, and which can transform prokaryotic or eukaryotic host either by integration into the cellular genome or exist extrachromosomally (e.g. autonomous replicating plasmid with an origin of replication).

Specifically included are shuttle vectors by which is meant a DNA vehicle capable, naturally or by design, of replication in two different host organisms, which may be selected from actinomycetes and related species, bacteria and eukaryotic (e.g. higher plant, mammalian, yeast or fungal cells). Preferably the nucleic acid in the vector is under the control of, and operably linked to, an appropriate promoter or other regulatory elements for transcription in a host cell such as a microbial, e.g. bacterial, or plant cell. The vector may be a bi-functional expression vector which functions in multiple hosts. In the case of genomic DNA, this may contain its own promoter or other regulatory elements and in the case of cDNA this may be under the control of an appropriate promoter or other regulatory elements for expression in the host cell.

“Wild-type” refers to the normal gene, virus, or organism found in nature without any known mutation.

### BRIEF DESCRIPTION OF THE FIGURES AND SEQUENCES

Figures:

FIG. 1 shows the insertion of the GFP expression cassette into the GAL polylinker of pBIN19.

Sequence Listing:

SEQ ID NOs: 1 to 342 provide the rice polynucleotide sequences according to the invention and the protein sequences which are encoded by the said polynucleotides in an alternating arrangement. The nucleic acid sequences are represented by the odd SEQ ID NOs: 1, 3, 5 . . . , and so on up to SEQ ID NO: 341, and the polypeptide sequences by the even SEQ ID NOs: 2, 4, 6, . . . and so on up to SEQ ID NO: 342, with the respective polypepdide immediately following its corresponding nucleic acid.

SEQ ID NOs: 343 to 472 provide rice cDNA sequences.

SEQ ID NOs: 473 to 539 provide banana cDNA/EST sequences which show homology to the rice sequences according to the invention.

SEQ ID NOs: 540 to 673 provide wheat cDNA/EST sequences which show homology to the rice sequences according to the invention.

SEQ ID NOs: 674 to 779 provide maize cDNA/EST sequences which show homology to the rice sequences according to the invention.

### DETAILED DESCRIPTION OF THE INVENTION

The elucidation of gene silencing mechanisms can lead to more efficiently expressed transgenes. It will also lead to a better understanding of plant-viral interactions, and to new methods of targeting the suppression of specific plant genes.

To identify genes important for posttranscriptional gene silencing (PTGS), the RNA expression patterns in high-expressing and silent Arabidopsis plants may be compared using DNA microarray technology. Arabidopsis plants transformed with a green fluorescent protein (GFP) reporter gene regulated by the cauliflower mosaic virus 35S RNA promoter (P35S) are used as the inhibition of GFP accumulation is a marker for PTGS. This system has the advantage that expression of the foreign GFP gene has little effect on the physiology of the plant. Accordingly, use of this method greatly increases the range of PTGS-related genes that can be identified. Use of this system also provides nucleic acid segments and corresponding polypeptides that are modulated by PTGS.

Several candidate proteins have recently been reported. A RecQ DNA helicase that is required for gene silencing in Neurospora has recently been described (Cogoni et al., Science 286:2342 (1999)). The DNA helicase may act by unwinding DNA, inducing changes in DNA methylation or chromatin structure that could result in the production of aberrant RNA. Another candidate is rgs-CaM, a calmodulin-related protein that suppresses plant post-transcriptional gene silencing. The involvement of this protein suggests that gene silencing may be regulated by Ca⁺⁺ binding activity (Anandalakshmi et al., Science 290:142 (2000). Another suggested candidate is RNA-dependent RNA polymerase, which might serve to produce antisense RNAs from sense transcripts (Jorgensen et al., Science 279 1486 (1998)). Since gene silencing is also induced by RNA viruses and involves aberrant DNA methylation, future research will involve overexpression or underexpression of candidate proteins combined with the use of the powerful research tools such as viral suppressors of gene silencing (Jones et al., Plant Cell 11:2291 (1999)).

The invention discloses a method to identify cell expression products that are modulated by posttranscriptional gene silencing (PTGS) and the products identified through use of the method. The invention further discloses a method of determining the siliencing status of a plant. Any one of the genes disclosed herein, but, preferably, subsets of genes comprising at least two or more genes the expression of which is modulated by gene silencing, can be used in said method.

Preferred subsets of genes that may be used in a method of determining the siliencing status of a plant comprise polynucleotides encoding polypeptides that are involved in RNA and DNA metabolism (e.g., RNA helicases, RNAses, reverse transcriptase, histones, histone acetyltransferases); signal transduction (e.g., protein kinases, receptors, and calmodulin); further transcription factors and biotic and abiotic stress-related proteins; and proteins involved in general metabolism.

Preferred is a subset of genes comprising polynucleotides encoding polypeptides that are involved in RNA and DNA metabolism and have at least 70% nucleotide sequence identity to the polynucleotides selected from the group consisting of SEQ ID NOs: 278, 92; 152, 142, and 244.

Further preferred is a subset of genes comprising polynucleotides encoding polypeptides that are involved in signal transduction and have at least 70% nucleotide sequence identity to the polynucleotides selected from the group consisting of SEQ ID NOs: 252, 4, 289, 55, 312, 40; 338; 132; and 180.

Also preferred is a subset of genes comprising polynucleotides encoding DNA-binding proteins and having at least 70% nucleotide sequence identity to the polynucleotides selected from the group consisting of SEQ ID NOs: 196, 64; 328, 140; 66, 264; 280, 198; 176, 22; 318, 58; 216, 116; 152, 142; 162, 46, 270, 12, 20, 200, 304 and 188.

Also preferred is a subset of genes comprising polynucleotides encoding polypeptides that are involved in the biotic and abiotic stress response of plants and have at least 70% nucleotide sequence identity to the polynucleotides selected from the group consisting of SEQ ID NOs: 174; 234; 18; 226, 68; 32, 182; 168, 60; 212, 72; 184, 108; 230, 42; 38, 322; 178, 24; 86; 204; 146; 320, 16, 316, 110, 106, 222, 238, 282, 102, 150, 94, and 186.

Further preferred is a subset of genes comprising polynucleotides encoding polypepetides with yet an unknown function and having at least 70% nucleotide sequence identity to the polynucleotides selected from the group consisting of SEQ ID NOs: 310, 30; 160, 50; 284; 296, 122; 242, 6; 164, 130; 280, 78; 218, 118; 336; 220, 76; 156, and 342.

The method according to the invention comprises obtaining the RNA expression profile for a gene or, preferably, a subset of genes the expression of which is modulated by posttranscriptional gene silencing (PTGS) and comparing the so obtained expression profile with the profile of a plant of the same species that does not have posttranscriptional gene silencing.

For determining the silencing status of a plant the expression profile of a single gene known to be modulated by PTGS may be obtained, but preferably of a group of 2, 3, 4, 5 and up to 20 and more genes. The said genes are preferably those provided in the above-defined subgroups with any combination of genes within said subgroups also being part of the invention.

In a further embodiment of the invention newly arranged subsets of genes may be used within the method according to the invention comprising 2, 3, 4, 5 and up to 20 and more genes obtained from two or more of the above defined subgroups.

The above methods involve comparing the contents of a cell that has posttranscriptional gene silencing with the contents of a cell of the same species that does not have posttranscriptional gene silencing. Cells which exhibit, and which do not exhibit, posttranscriptional gene silencing may be identified through use of a marker gene that is operatively linked to a constitutive promoter. One non-limiting example of such a marker gene is green fluorescent protein (GFP). Preferably the marker gene has little physiological effect on the cell. Cells exhibiting posttranscriptional gene silencing may be identified by inhibited accumulation of the marker gene product (i.e. GFP). This method can be adapted for use with many cells and it is understood that the exemplary use of Arabidopsis disclosed in the present invention is not to be limiting in any way. Preferably the method can be used with plant cells. More preferably the method can be used with the cells of plants used in agriculture. Most preferably the method can be used with the cells of commercial plants such as rice.

In one embodiment of the invention two cells of the same species, one which exhibits PTGS and one which does not, are grown under the same conditions. Plant tissues are collected and RNA is extracted through use of a method or methods known in the art and used to prepare biotinylated cRNA probes (Sambrook et al.,1989; Example II; http://afgc.stanford.edu/afgc, htm1/site2Rna.htm#pinetree). Briefly, RNA can be prepared through use of an RNeasy column and then precipitated overnight at −20° C. after the addition of 0.25 volumes of 10M LiCl₂ and pelleting by centrifugation. The pellet is washed with 70% EtOH, air dried and resuspended in RNase-free (DEPC-treated) water. The RNA produced is used to prepare cDNA by annealing an oligo dT₍₂₄₎ primer, containing a 5′ T7 RNA polymerase promoter sequence, to RNA isolated from plant tissue and adding reverse transcriptase to cause first strand cDNA synthesis. Second strand cDNA synthesis can then be performed using E. coli DNA polymerase, ligase and RNase H. The cDNA products are then purified by phenol/chloroform extraction and EtOH precipitation. Biotinylated cRNA probes can be prepared through in vitro transcription using T7 RNA polymerase (ENZO BioArray High Yield RNA Transcript Labeling Kit).

The biotinylated probes may then be fragmented through chemical or mechanical means and annealed to a oligonucleotide array (see Example II). The array may then be scanned with a Hewlett-Packard GeneArray scanner and the expression level of the individual genes contained on the array (Affymetrix, Santa Clara, Calif.) is determined and compared to determine genes modulated by posttranscriptional gene silencing.

In another embodiment, cDNA-AFLP (complimentary deoxyribonucleic acid-amplified fragment length polymorphism) can be used to identify nucleic acid sequences modulated by posttranscriptional gene silencing within a cell. Durrant et al., The Plant Cell, 12:963 (2000). Briefly, total RNA is extracted from the tissues of at least two cells, as described above, and further purified with an mRNA purification system to isolate polyA-mRNA (Oligotex mRNA purification system, Qiagen Inc., Valencia, Calif.). PolyA-mRNA may also be isolated according to other methods well known in the art (Sambrook et al., 1989). However, total RNA may also be used in lieu of mRNA. The isolated mRNA is used to generate cDNA through methods well known in the art (Life Technologies, Rockville, Md.). Briefly, an oligo(dT) primer may be annealed to the mRNA and first strand cDNA synthesis reactions can be performed with SuperScript II reverse transcriptase (RT) (Gibco/BRL) according to the manufacturer's recommendations using 50 mM Tris-HCl (pH 8.3), 75 mM KCl, 3mM MgCl₂, 10 mM dithiotreitol (DTT), 0.5 mM dNTPs, and 200 units of RT enzyme. The second cDNA strand may be synthesized using 40 units of E. coli DNA polymerase, 10 units of E. coli ligase, and 2 units of RNase H in a reaction containing 25 mM Tris-HCl (pH 7.5), 100 mM KCl, 5 mM MgCl₂, 10 mM (NH₄)SO₄, 0.15 mM β-NAD⁺, 1 mM dNTPs, and 1.2 mM DTT. The reaction proceeds at 16° C. for 2 hours and is terminated with EDTA. Double-stranded cDNA products may be purified by phenol/chloroform extraction and ethanol precipitation.

The cDNA is digested with two different restriction enzymes and linkers having complimentary ends are ligated onto the ends of the digested cDNA fragment. Two primers are combined with the ligated cDNA product. Each of the primers anneals to each of the linkers that were ligated to the cDNA fragment such that a PCR reaction carried on between the two annealed primers will amplify the intervening sequence corresponding to that of the cDNA fragment.

The products of the PCR reaction can be separated on a polyacrylamide gel and the products quantitated through means well known in the art. Examples of such means are labeling of the products with fluorescent tags, radioactivity, antibody based systems or the like. This is followed by use of a fluorescence scanner, autoradiography or other detection method known in the art (Sambrook et al., 1989; Applied Biosystems, Foster City, Calif.; Beckman Coulter, Fullerton, Calif.).

Sequences that are up-modulated or down-modulated by posttranscriptional gene silencing within a cell can be identified by comparing the intensity of a sequence from a cell having PTGS to that of a cell which does not have PTGS. Identified bands are then excised from the gel and sequenced. The determined sequence is then compared to known sequences or used as a probe to determine the full length sequence of a gene that is modulated by posttranscriptional gene silencing.

Additionally, differential display and cDNA fingerprinting can be used to identify genes modulated by posttranscriptional gene silencing. (CuraGen Corp., New Haven, Conn., Digital Gene Tech., LaJolla, Calif.; Liang et al., Science, 257:967 (1992)).

Furthermore, metabolic and protein profiling methods such as mass spectroscopy and 2-dimensional gel electrophoresis can be used to identify proteins that are modulated by PTGS. These proteins can then be sequenced and reverse genetics may be used to isolate the corresponding gene that is modulated by posttranscriptional gene silencing. Briefly, a polypeptide that is modulated by posttranscriptional gene silencing is isolated and the amino acid sequence is determined through methods known in the art, such as chemical cleavage (Edman degradation) or through protease based sequencing methods. A codon table and synthetic methods known in the art (Sambrook et al, 1989) can be used to prepare a probe that will anneal to the gene that encodes the polypeptide, thus allowing the gene to be isolated according to standard methods.

The Arabidopsis sequences can be compared to different sets or rice sequences to identify homologs. One such set may contain rice gene prediction mRNA sequences and another set may contain other rice cDNA ORF sequences. The comparison algorithm can then be used such as, for example, a translated BLAST search, tblastx. The BLAST results can be post processed using an appropriate software such as, for example, SCAN software with the default parameters. This processed data may then be parsed to retrieve the top rice hit based on E-value. These rice sequences can then be compared to sets of clustered cDNA's from other cereal species such as, for example, wheat and banana using the same software process to retrieve the top hits from each set.

Thus, the present invention, in an embodiment applicable to all of the above stated provisions, provides nucleotide sequences corresponding to genes modulated within a cell by posttranscriptional gene silencing from cereals, but especially from rice, representative examples of which are identified in Tables 1 and 2 and listed in the sequence listing encoding at least one polypeptide involved in transcriptional proteins and/or activities, as well as the polypeptide encoded thereby, or an antigene sequence thereof, which have numerous applications using techniques that are known to those skilled in the art of molecular biology, biotechnology, biochemistry, genetics, physiology or pathology. These techniques include the use of nucleotide molecules as hybridization probes, for chromosome and gene mapping, in PCR technologies, in the production of sense or antisense nucleic acids, in screening for new therapeutic molecules, in production of plants and seeds having desirable, inheritable, commercially useful phenotypes, or in discovery of inhibitory compounds.

In a further embodiment, the present invention provides the ability to modulate transcription, by over-expressing, under-expressing or knocking out one or more genes involved in transcription chaperones,DNA binding factors and transcription factors, chromatin modification, and gene silencing genes, or their gene products, in a host cell, preferably in a plant cell, in vitro or in planta. Expression vectors including at least one nucleotide sequence involved in said sequences, or its antigene, operably linked to at least one suitable promoter and/or regulatory sequence can be used to study the role of polypeptides encoded by said sequences, for example by transforming a host cell with said expression vector and measuring the effects of overexpression and underexpression of sequences.

In particular, the present invention also provides isolated polynucleotide segments that are modulated within a cell by posttranscriptional gene silencing. (see Tables 1-2). These segments include, but are not limited to, those polynucleotide segments corresponding to genes modulated within a cell by posttranscriptional gene silencing from cereals, but especially from rice, identified in Tables 1 and 2 and listed in the sequence listing and sequences having at least 70% nucleotide sequence identity to the polynucleotide segments listed in the sequence listing. The polynucleotide segments of the invention also include mutations of the sequences corresponding to genes modulated within a cell by posttranscriptional gene silencing from cereals, but especially from rice, identified in Tables 1 and 2 and listed in the sequence listing that encode for the same amino acids due to the degeneracy of the genetic code. For example, the amino acid threonine is encoded by ACU, ACC, ACA and ACG. It is intended that the invention includes all variations of the polynucleotide segments corresponding to genes modulated within a cell by posttranscriptional gene silencing from cereals, but especially from rice, identified in Tables 1 and 2 and in the sequence listing that encode for the same amino acids. Such mutations are known in the art (Watson et al, Molecular Biology of the Gene, Benjamin Cummings 1987). Mutations also include alteration of a polynucleotide segments to encode for conservative amino acid changes.

Such amino acid changes are exemplified by the following five groups which contain amino acids that are conservative substitutions for one another: Aliphatic: Glycine (G), Alanine (A), Valine (V), Leucine (L), Isoleucine (I); Aromatic: Phenylalanine (F), Tyrosine (Y), Tryptophan (W); Sulfur-containing: Methionine (M), Cysteine (C); Basic: Arginine (R), Lysine (K), Histidine (H); Acidic: Aspartic acid (D), Glutamic acid (E), Asparagine (N), Glutamine (Q). See also, Creighton, 1984. Thus, the genes and nucleotide sequences of the invention include both the naturally occurring sequences as well as mutant forms.

In a specific embodiment, the nucleic acid sequences of the invention encode polypeptides involved with silencing-related RNA and DNA metabolism (e.g., RNA helicases, RNAses, reverse transcriptase, histones, histone acetyltransferases); signal transduction (e.g., protein kinases, receptors, and calmodulin); transcription factors; stress-related and pathogen-related proteins; and general metabolism. More prefered are reverse transcribase-like protein and histone acetyltransferase-like protein.

Silencing-related RNA and DNA Metabolism (e.g., RNA Helicases, RNAses, Reverse Transcriptase, Histones, Histone Acetyltransferases)

In a further specific embodiment the invention relates to an isolated polynucleotide segment that is modulated within a cell by posttranscriptional gene silencing which polynucleotide sequence encodes a histone acetyltransferase-like protein but, preferably, a polynucleotide segment encoding a protein as given in any one of SEQ ID NOs: 278, 92; and 152, 142, respectively including sequences having at least 70% nucleotide sequence identity to the polynucleotide segments listed in the sequence listing.

Acetylation and deacetylation of histones is believed to be one important mechanism for the dynamic alteration of chromatin structure, which is affected by two enzyme activities, histone acetyltransferase (HAT) and histone deacetylase (HD). Biochemical studies have revealed a correlation between the level of histone acetylation and deacetylation with transcriptional activity and repression respectively. It is thought that the acetylation of nucleosomal histones induces an open chromatin conformation, which allows the transcription machinery access to promoters.

The basic element of chromatin is the nucleosome. Histones H4, H3, H2A and H2B form the core histone octamer by protein-protein interactions of their folded domains. The most strictly conserved parts of core histones are the N-terminal extensions, which protrude from the nuclesome and contain numerous amino acids that are subject to posttranslational acetylation. Histone acetyltransferases (HATs) transfer the acetyl moiety of acetyl-coenzyme A to the epsilon-amino group; this reaction is reverted by histone deacetylases (HDACs). The dynamic equilibrium of the acetylation/deacetylation reaction varies throughout the genome; some regions in chromatin undergo rapid acetylation/deacetylation, whereas others are fixed in a certain acetylation state without significant changes.

Hence, histone acetylation and deacetylation are considered fundamental regulatory mechanisms governing cell proliferation and differentiation transcriptional regulation including DNA replication, chromatin remodelling, DNA repair; etc (for reviews see Wade and Wolffe, Curr. Biol. 7:82-84, 1997; Wolffe, Nature 387:16-17, 1997).

In a further specific embodiment the invention relates to an isolated polynucleotide segment that is modulated within a cell by posttranscriptional gene silencing which polynucleotide sequence encodes a reverse transcriptase protein but, preferably, a polynucleotide segment encoding a protein as given in SEQ ID NO: 244 including sequences having at least 70% nucleotide sequence identity to the polynucleotide segments listed in the sequence listing.

Reverse transcriptase (RT) is a modular enzyme carrying polymerase and ribonuclease H (RNase H) activities in separable domains. Reverse transcriptase (RT) converts the single-stranded RNA genome of a retrovirus into a double-stranded DNA copy for integration into the host genome. This process requires ribonuclease H as well as RNA-and DNA-directed DNA polymerase activities. Retroviral RNase H is synthesised as part of the POL polyprotein that contains; an aspartyl protease, a reverse transcriptase, RNase H and integrase. POL polyprotein undergoes specific enzymatic cleavage to yield the mature proteins. Bacterial RNase H (catalyses endonucleolytic cleavage to 5′-phosphomonoester acting on RNA-DNA hybrids.

Signal Transduction (e.g. Protein Kinases, Receptors, and Calmodulin)

The present invention further provides at least one nucleotide sequence encoding at least one polypeptide involved in signal transduction including receptor proteins, second messengers and G proteins, or any antigene sequences thereof. Receptor proteins play a role in the initial perception of changes in abiotic and biotic environmental factors such as light, nutrient availability, drought, salt, and pathogen attack. Second messengers and mediator proteins are involved in the perception and transduction of many signals from the perception site (often the plasma membrane) to target sites in the cell. G proteins are involved in many diverse responses in both plants and animals, such as responses to transduction of blue light, red light, auxin, giberellin, and stomatal opening (Trewavas, Signal perception and transduction in Buchanan, supra). Thus, these key groups of proteins are critical regulators of signal systems in plants, and nucleotide sequences encoding at least one polypeptide involved in receptor proteins, second messengers and G proteins, as well as the polypeptide encoded thereby, or any antigene sequences thereof, are commercially useful materials that can be used to study these processes and to modify these processes to elicit desired changes.

In a preferred embodiment the present invention relates to an isolated polynucleotide segment that is modulated within a cell by posttranscriptional gene silencing which polynucleotide sequence encodes a calmodulin-related protein but, preferably, a polynucleotide segment encoding a protein as given in SEQ ID NO: 252 and 4, respectively including sequences having at least 70% nucleotide sequence identity to the polynucleotide segments listed in the sequence listing.

Calmodulin, the principle Ca-binding protein in plants, mediates the messenger function of Ca+ ions. This highly conserved, soluble protein is found in the cytosol of plants and animals. Once bound, the Ca-calmodulin complex binds to protein kinases, which are in turn activated. Although the function of calmodulin in plants has not been thoroughly investigated (Heldt (1999) supra), it is thought that the protein will occupy a similar role in both plant and animal cells. Recent studies have also shown that calcium may play an important role in gravitropic responses (Raven et al., (1999) supra; Perdue et al., Plant Physiology 86: 1276 (1988)) of shoots and roots.

Further preferred is an isolated polynucleotide segment that is modulated within a cell by posttranscriptional gene silencing which polynucleotide sequence encodes a protein that shows similarity to the calcium-binding protein annexin, but, preferably, a polynucleotide segment encoding a protein as given in SEQ ID NOs: 289 and 55, respectively including sequences having at least 70% nucleotide sequence identity to the polynucleotide segments listed in the sequence listing.

In another preferred embodiment the, the present invention relates to an isolated polynucleotide segment that is modulated within a cell by posttranscriptional gene silencing which polynucleotide sequence encodes a protein kinase, including a receptor-linked protein kinase, but preferably, a polynucleotide segment encoding a protein as given in any one of SEQ ID NOs: 312, 40; and, 338; 132; and 180, respectively including sequences having at least 70% nucleotide sequence identity to the polynucleotide segments listed in the sequence listing.

Receptor-linked protein kinases are responsible for regulating cell wall components and are involved in senescence and a host of other phytohormone and resistance factors. As such, they provide the potential to regulate fiber modifications and lysine-rich proteins in crop plants, and additionally pose application in the control of crops by regulating pollen and ovule development (PRK-1s). Receptor linked protein kinases have an extracellular ligand-binding domain, but only have one membrane spanning region (Trewavas, Signal perception and transduction, in Buchanan, supra). Additionally, they have a kinase activation site on the cytosolic side of the protein, which is putatively activated by ligand binding. The protein exists as a dimer, so the ligand-binding causes the cytosolic region of the dimers to come in close proximity and be activated and stabilized by phosphorylation. The activated receptor may be involved in the activation of other proteins.

DNA Binding Proteins/Transkription Factors

In another preferred embodiment, the present invention provides at least one nucleotide sequence encoding at least one regulator of transcription, including but not limited to transcription factors that modulate the level of transcription with respect to tissue specificity of transcription, transcriptional responses to particular environmental or nutritional factors, where such transcription factors may include but are not limited to, transcriptional activators, transinhibitors, repressors, co-repressors, gene activator proteins, integration host factors, and sigma factors.

In particular, the present Invention relates to at least one nucleotide sequence encoding at least one regulator of transcription as given in SEQ ID NOs:; 196, 64; 328, 140; 66, 264; 280, 198; 176, 22; 318, 58; 216, 116; 152, 142; 162, 46, 270, 12, 20, 200, 304 and 188 respectively including sequences having at least 70% nucleotide sequence identity to the polynucleotide segments listed in the sequence listing.

While many transcription factors bind to sites on DNA, not all transcription factors bind DNA directly. Some bind to another transcription factor or to a DNA-protein complex. Transcription factors that bind to other proteins can be isolated using a cross-linking agents or a two-hybrid “interaction trap” system. Transcription factors that bind specific DNA sequences can be isolated using affinity tags or specifically designed oligonucleotides to identify transcription factors in cell extracts and the DNA-protein complex isolated using gel-shift techniques and purified for PCR amplification. Transcription factors that directly bind DNA may bind to sites in promoter or enhancer regions, where the primary role of enhancers is not simply to provide additional transcription factors to facilitate formation of an active initiation complex but to relieve repression of weak promoters due to chromatin structure.

In a further embodiment, the nucleotide sequence of the present invention binds to, or encodes a polypeptide that can bind to, a key site in an operon, regulon or other target sequence to effect regulation of the expression and function of a group of coordinately regulated genes.

In particular, the present invention relates to an isolated polynucleotide segment that is modulated within a cell by posttranscriptional gene silencing which polynucleotide sequence encodes a DNA-binding protein including a RAV-like domain DNA-binding protein, but preferably, a polynucleotide segment encoding a protein as given in SEQ ID NO: 318 and 58, respectively including sequences having at least 70% nucleotide sequence identity to the polynucleotide segments listed in the sequence listing.

The RAV-like B3 domain DN-binding proteins belong to a family of plant transcription factors which have various roles in development. The aligned region corresponds the B3 DNA binding domain. This domain is found in VP1/AB13 transcription factors. Some proteins also have a second AP2 DNA binding domain AP2-domain such as RAV1 Q9ZWM9. (Kagaya et al., Nucleic Acids Res, 27:470-478 (1999); Ulmasov et al., Science, 276:1865-1868 (1997)).

Another embodiment of the invention relates to an isolated polynucleotide segment that is modulated within a cell by posttranscriptional gene silencing which polynucleotide sequence encodes a WRKY domain transcription factor, but preferably, to a polynucleotide segment encoding a protein as given in SEQ ID NO: 196 and 64, respectively including sequences having at least 70% nucleotide sequence identity to the polynucleotide segments listed in the sequence listing.

Several examples of agronomically important DNA-binding domains have been investigated. Plants are composed of various cell and tissue types, all of which require different gene expression patterns. Gene expression requirements will undoubtedly change throughout development of the plant, as a tissue moves through the stages of biogenesis, maturity, and senescence. The WRKY DNA binding domain has been characterized in a number of plants including rice, parsley, wild oat, sweet potato, turnip and cucumber, as well as Arabidopsis, in which 100 representatives have been found. The proteins of this domain belong to a broad class and are involved in processes as unrelated as hormonal regulation, sucrose related gene expression, trichome development, and defense mechanisms. One novel characteristic of this domain is the presence of a C2-H2 zinc finger that has a distinctive spacing between the histidine and cysteine residues. In spite of the overall diversity of protein structure in this group, and the possibility for equally diverse functions, the WRKY domain transcription factors are strongly implicated in the regulation of early defense-response genes, particularly with regard to fungal pathogens.

In another preferred embodiment the present invention relates to an isolated polynucleotide segment that is modulated within a cell by posttranscriptional gene silencing which polynucleotide sequence encodes a zinc finger-type protein such as the lsd1-type protein or the CCCH-type protein, but preferably, a polynucleotide segment encoding a protein as given in SEQ ID NOs: 152, 66, 20, 200, and 188 respectively including sequences having at least 70% nucleotide sequence identity to the polynucleotide segments listed in the sequence listing.

The Lsd-1 gene encodes a zinc finger protein and is involved in the regulation of cell death. It acts as a gatekeeper, or “negative regulator.” in the plant cell; that is, it keeps cell death turned off until it receives the right signalIn Arabidopsis, LSD1 is required to prevent the programmed cell death response characteristic of gene-for-gene resistance from spreading beyond the site of infection and killing the entire plant. It encodes a zinc-finger protein (Dietrich et al., Cell 88:685-694 (1997)).

Zinc finger domains are thought to be involved in DNA-binding, and exist as different types, depending on the positions of the cysteine residues. Proteins containing zinc finger domains of the C-x8-C-x5-C-x3-H type include zinc finger proteins from eukaryotes involved in cell cycle or growth phase-related regulation, e.g. human TIS11B (butyrate response factor 1), a probable regulatory protein involved in regulating the response to growth factors, and the mouse TTP growth factor-inducible nuclear protein, which has the same function. The mouse TTP protein is induced by growth factors. Another protein containing this domain is the human splicing factor U2AF 35 kD subunit, which plays a critical role in both constitutive and enhancer-dependent splicing by mediating essential protein-protein interactions and protein-RNA interactions required for 3′ splice site selection. It has been shown that different CCCH zinc finger proteins interact with the 3′ untranslated region of various mRNA. This type of zinc finger is very often present in two copies.

In still another preferred embodiment the present invention relates to an isolated polynucleotide segment that is modulated within a cell by posttranscriptional gene silencing which polynucleotide sequence encodes an AP2-domain transcription factor protein, but preferably, a polynucleotide segment encoding a protein as given in any one of SEQ ID NOs: 140, and 176, 22and 270, 12, respectively including sequences having at least 70% nucleotide sequence identity to the polynucleotide segments listed in the sequence listing.

This 60 amino acid residue AP2-domain can bind to DNA. This domain is plant specific. Members of this family are suggested to be related to pyridoxal phosphate-binding domains such as found in aminotran 2 Ethylene, chemically the simplest plant hormone, participates in a number of stress responses and developmental processes: e.g., fruit ripening, inhibition of stem and root elongation, promotion of seed germination and flowering, senescence of leaves and flowers, and sex determination. DNA sequence elements that confer ethylene responsiveness have been shown to contain two 11 bp GCC boxes, which are necessary and sufficient for transcriptional control by ethylene. Ethylene responsive element binding proteins (EREBPs) have now been identified in a variety of plants. The proteins share a similar domain of around 59 amino acids, which interacts directly with the GCC box in the ERE. (Ohme-takagi et al., Plant Cell, 7:173-182 (1995); Weigel, Plant Cell, 7:388-389 (1995); Mushegian et al., Genetics, 144:817-828 (1996)). In still another preferred embodiment the present invention relates to an isolated polynucleotide segment that is modulated within a cell by posttranscriptional gene silencing which polynucleotide sequence encodes an MADS-box transcription factor protein, but preferably, a polynucleotide segment encoding a protein as given in SEQ ID NOs: 304, and 162, 46, respectively including sequences having at least 70% nucleotide sequence identity to the polynucleotide segments listed in the sequence listing. Proteins belonging to the MADS family function as dimers, the primary DNA-binding element of which is an anti-parallel coiled coil of two amphipathic alpha-helices, one from each subunit. The DNA wraps around the coiled coil allowing the basic N-termini of the helices to fit into the DNA major groove. The chain extending from the helix N-termini reaches over the DNA backbone and penetrates into the minor groove. A 4-stranded, anti-parallel beta-sheet packs against the coiled-coil face opposite the DNA and is the central element of the dimerisation interface. The MADS-box domain is commonly found associated with K-box region.

Stress-related and Pathogen-related Proteins

In another preferred embodiment the present invention relates to an isolated polynucleotide segment that is modulated within a cell by posttranscriptional gene silencing which polynucleotide sequence encodes a protein that is involved in the plants response to biotic or abiotic stresses, but preferably, to a polynucleotide segment encoding a protein as given in any one of SEQ ID NOs: 174; 234; 18; 226, 68; 32, 182; 168, 60; 212, 72; 184, 108; 230, 42; 38,322; 178, 24; 86; 204; 146; 320, 16, 316, 110, 106, 222, 238, 282, 102, 150, 94, 235, 1, 316, 110, and 186 respectively including sequences having at least 70% nucleotide sequence identity to the polynucleotide segments listed in the sequence listing.

In a preferred embodiment, the present invention relates to an isolated polynucleotide segment that is modulated within a cell by posttranscriptional gene silencing which polynucleotide sequence encodes a cytochrome P450 protein, but preferably, to a polynucleotide segment encoding a protein as given in any one of SEQ ID NOs: 234, 32, 182;146, 282, and 102, respectively including sequences having at least 70% nucleotide sequence identity to the polynucleotide segments listed in the sequence listing. Cytochrome P450s are involved in the oxidative degradation of various compounds, and are particularly well known for their role in the degradation of environmental toxins and mutagens. Structure is mostly alpha, and binds a heme cofactor. The cytochrome P450 enzymes usually act as terminal oxidases in multicomponent electron transfer chains, called P450-containing monooxygenase systems. P450-containing monooxygenase systems primarily fall into two major classes: bacterial/mitochondrial (type I), and microsomal (type II). All P450 enzymes can be categorised into two main groups, the so-called B- and E-classes: P450 proteins of prokaryotic 3-component systems and fungal P450nor (CYP55) belong to the B-class; all other known P450 proteins from distinct systems are of the E-class. This family contains a number of subtypes of both B and E classes.

In another preferred embodiment, the present invention relates to an isolated polynucleotide segment that is modulated within a cell by posttranscriptional gene silencing which polynucleotide sequence encodes a glucanase or anendochitinase or a class I or IV chitinase, but, preferably, to a polynucleotide segment encoding a protein as given in any one of SEQ ID NOs: and 222, 106, 226, 68; 212, 72 and 238, respectively including sequences having at least 70% nucleotide sequence identity to the polynucleotide segments listed in the sequence listing.

Chitinases (EC 3.2.1.14) are enzymes that catalyze the hydrolysis of the beta-1,4-N-acetyl-D-glucosamine linkages in chitin polymers. From the view point of sequence similarity chitinases belong to either family 18 or 19 in the classification of glycosyl hydrolases. Chitinases of family 18 (also known as classes III or V) groups a variety of chitinases and other proteins. Site-directed mutagenesis experiments and crystallographic data, have shown that a conserved glutamate is involved in the catalytic mechanism and probably acts as a proton donor. This glutamate is at the extremity of the best conserved region in these proteins.

In still another preferred embodiment, the present invention relates to an isolated polynucleotide segment that is modulated within a cell by posttranscriptional gene silencing which polynucleotide sequence encodes an peroxidase protein, but, preferably, to a polynucleotide segment encoding a protein as given in any one of SEQ ID NOs: 314, 62; 18; 168, 60; 186; 320, and 16 including sequences having at least 70% nucleotide sequence identity to the polynucleotide segments listed in the sequence listing.

Peroxidases are haem-containing enzymes that use hydrogen peroxide as the electron acceptor to catalyse a number of oxidative reactions. Most haem peroxidases follow the reaction scheme in which the enzyme reacts with one equivalent of H₂O₂ to give [Fe⁴⁺═O]R′ (compound I). This is a two-electron oxidation/reduction reaction where H₂O₂ is reduced to water and the enzyme is oxidised. One oxidising equivalent resides on iron, giving the oxyferryl intermediate, while in many peroxidases the porphyrin (R) is oxidised to the porphyrin pi-cation radical (R′). Compound I then oxidises an organic substrate to give a substrate radical. Peroxidases are found in bacteria, fungi, plants and animals and can be viewed as members of a superfamily consisting of 3 major classes. Class I, the intracellular peroxidases, includes: yeast cytochrome c peroxidase (CCP), a soluble protein found in the mitochondrial electron transport chain, where it probably protects against toxic peroxides; ascorbate peroxidase (AP), the main enzyme responsible for hydrogen peroxide removal in chloroplasts and cytosol of higher plants, and bacterial catalase-peroxidases, exhibiting both peroxidase and catalase activities. It is thought that catalase-peroxidase provides protection to cells under oxidative stress. Class II consists of secretory fungal peroxidases: ligninases, or lignin peroxidases (LiPs), and manganese-dependent peroxidases (MnPs). These are monomeric glycoproteins involved in the degradation of lignin. In MnP, Mn (2+) serves as the reducing substrate. Class II proteins contain four conserved disulphide bridges and two conserved calcium-binding sites. Class III consists of the secretory plant peroxidases, which have multiple tissue-specific functions: e.g., removal of hydrogen peroxide from chloroplasts and cytosol; oxidation of toxic compounds; biosynthesis of the cell wall; defence responses towards wounding; indole-3-acetic acid (IAA) catabolism; ethylene biosynthesis; and so on. Class III proteins are also monomeric glycoproteins, containing four conserved disulphide bridges and two calcium ions, although the placement of the disulphides differs from class II enzymes. The crystal structures of a number of these proteins show that they share the same architecture—two all-alpha domains between which the haem group is embedded.

In still another preferred embodiment, the present invention relates to an isolated polynucleotide segment that is modulated within a cell by posttranscriptional gene silencing which polynucleotide sequence encodes germin oxalate oxidase protein, but, preferably, to a polynucleotide segment encoding a protein as given in SEQ ID NOs: 235 and 1, respectively, including sequences having at least 70% nucleotide sequence identity to the polynucleotide segments listed in the sequence listing.

Germin and its relatives from barley are cereal glycoproteins expressed during germination, they are oxalate oxidase enzymes EC:1.2.3.4. The three conserved histidine residues are the ligands for the active site metal, a single manganese atom. The enzyme is a homohexamer. The structure of this family is predicted to be a beta-barrel protein based on similarity to the known structures of related Seedstore 7s and Seedstore 11s. These storage proteins are duplicated versions of germin, ie they have two linked beta-barrels whereas germin has a single domain. This family is a member of the ‘cupin’ superfamily on the basis of their conserved barrel domain (‘cupa’ is the Latin term for a small barrel). Germins are a family of homopentameric cereal glycoproteins expressed during germination which may play a role in altering the properties of cell walls during germinative growth. It has been shown that wheat and barley germins act as oxalate oxidases (EC:1.2.3.4), an enzyme that catalyzes the oxidative degradation of oxalate to carbonate and hydrogen peroxide. Germins are highly similar to Slime mold spherulins 1a and 1b and Germin-like proteins from various plants.

In still another preferred embodiment, the present invention relates to an isolated polynucleotide segment that is modulated within a cell by posttranscriptional gene silencing which polynucleotide sequence encodes an taumatin-like protein, but, preferably, to a polynucleotide segment encoding a protein as given in any one of SEQ ID NOs: 316 and 110, respectively, including sequences having at least 70% nucleotide sequence identity to the polynucleotide segments listed in the sequence listing.

The sweet-tasting protein thaumatin is a soluble protein located in the cytoplasm of the fruit cells of the tropical monocotyledon plant Thaumatococcus danielli Benth.,. Thaumatin shares amino acid sequence similarity with osmotin and some pathogenesis-related proteins. All these proteins have sixteen conserved cysteine residues that form eight disulfur bonds and share similar secondary structure. Thaumatin-like proteins have been detected in a variety of higher plants and their expression has been reported not only in response to phytopathogen infection but also during flower development, and fruit ripening.

Proteins with Unknown Function

Another important group of proteins comprises those proteins for which no function could be identified yet.

Within the scope of the present invention it could be demonstrated that the expression of these proteins is modulated within a cell by posttranscriptional gene silencing. An isolated polynucleotide segment that is modulated within a cell by posttranscriptional gene silencing which polynucleotide sequence encodes a protein within this group is provided in any one of SEQ ID NOs: 310, 30; 160, 50; 284; 296, 122; 242, 6; 164, 130; 280, 78; 218, 118; 336; 220, 76; 156, and 342, respectively including sequences having at least 70% nucleotide sequence identity to the polynucleotide segments listed in the sequence listing. These and the other polynucleotide sequence according to the invention identified hereinbefore can be used in a method to modulate gene expression by posttranscriptional gene silencing. The method involves transforming a cell with a polynucleic acid segment according to the invention that modulates gene expression by posttranscriptional gene silencing.

In accordance with the present invention, polypeptides encoded by the polynucleic acid segments of the invention and variants thereof are provided. These polypeptides are exemplified by those encoded by the nucleic acid sequences corresponding to genes modulated within a cell by posttranscriptional gene silencing from cereals, but especially from rice, identified in Tables 1 and 2 and listed in the sequence listing, polypeptides encoded by nucleic acid sequences having at least 70% sequence identity to the sequences in the sequence listing and variants and mutants thereof.

The polypeptides of the invention may be altered in various ways including amino acid substitutions, deletions, truncations, and insertions. Methods for such manipulations are generally known in the art. Methods for mutagenesis and nucleotide sequence alterations are well known in the art. See, for example, Kunkel, Proc. Natl. Acad. Sci. USA, 82:488, (1985); Kunkel et al., Methods in Enzymol., 154:367 (1987); U.S. Pat. No. 4,873,192; Walker and Gaastra, eds. (1983) Techniques in Molecular Biology (MacMillan Publishing Company, New York) and the references cited therein. Guidance as to appropriate amino acid substitutions that do not affect biological activity of the protein of interest may be found in the model of Dayhoff et al. (1978) Atlas of Protein Sequence and Structure (Natl. Biomed.Res. Found., Washington, D.C.), herein incorporated by reference. Conservative substitutions may be preferred.

The proteins of the invention encompass both naturally occurring polypeptides as well as variants and modified forms thereof. Obviously, the mutations that will be made in the DNA encoding the mutation must not place the sequence out of reading frame and preferably will not create complementary regions that could produce secondary mRNA structure. See, EP Patent Application Publication No. 75,444.

The invention also provides a method to determine the sequence of one or more regulatory elements that modulate gene expression within a cell by posttranscriptional gene silencing. In one embodiment, a genomic library may be probed with a polynucleic acid segment that is known to be modulated by posttranscriptional gene silencing within the cell in order to isolate the regulatory element that modulates expression of the gene. This procedure may be done according to methods known in the art (Sambrook et al., 1989). In one example meant for illustration and not to be limiting in any way, a chromosomal library of rice may be probed with a polynucleic acid segment described by in the sequence listing according to known hybridization techniques. Briefly, a bacterial artificial chromosome library containing rice genomic DNA may be constructed according to known methods (Choi et al, Plant Mol. Biol. Rep., 13:124 (1995)). A library such as this contains E. coli carrying single copy bacterial artificial chromosomes into which rice genomic DNA has been inserted. These inserts are approximately 150 kb in length. Filters or other supports known in the art are inoculated with the bacteria of the library. Colonies are formed on the support and the bacteria are lysed with the DNA becoming fixed to the support. The support may then be probed with a polynucleic acid segment corresponding to a gene that is modulated by posttranscriptional gene silencing. The nucleotide sequence of such a polynucleic acid segment is provided in the sequence listing. The polynucleic acid segment to be used as a probe may be labeled with radioactivity or other means know in the art, such as fluorescent groups, that allow the location of the probe to be determined. The probe is added to the support onto which the rice genomic DNA is fixed and allowed to anneal to the complementary nucleic acid sequence. The identity of the specific bacterial clone containing the rice genomic DNA to which the probe annealed is determined from the position of the probe as determined by radiography or other methods. This allows the bacterial artificial chromosome to be isolated from the selected bacterial clone and sequenced to determine elements regulated in response to posttranscriptional gene silencing. The isolated elements may be inserted into a construct containing a reporter gene, such as chloramphenicol acetyl transferase (CAT), luciferase, 9-glucuronidase (GUS) or green fluorescent protein (GFP), and then introduced into an appropriate cell to confirm the regulatory role of the element. GUS expression vectors and GUS gene cassettes are available from Clonetech Laboratories, Inc., Palo Alto, Calif. while luciferase expression vectors and luciferase gene cassettes are available from Promega Corp. (Madison, Wis.).

In another example, thermal asymmetric interlaced polymerase chain reaction (TAIL-PCR) may be used to identify elements that regulate gene expression by posttranscriptional gene silencing. Terauchi et al., Mol. Gen. Genet., 263:554 (2000). Briefly, total genomic DNA is extracted from a plant using methods known in the art, such as the CTAB (cetlytrimethylammonium bromide) method (Murray et al., Nuc. Acid. Res., 8:4321 (1980)). A primer is obtained that specifically anneals to a rice gene that is modulated by posttranscriptional gene silencing, such as those described in the sequence listing, and a degenerate primer or primers are constructed that will anneal to random nucleic acid sequences. The primers are constructed such that a PCR reaction conducted with the specific primer and the degenerate primer will amplify the region 5′ to the gene that is modulated within a cell by posttranscriptional gene silencing. The fragment produced can then be inserted into a vector and sequenced according to methods known in the art. In this way, regulatory elements that are 5′ of the gene modulated within a plant cell by posttranscriptional gene silencing can be identified. This method can also be used for identification of elements located 3′ of the PTGS-modulated gene. The regulatory role of elements isolated in this manner can be inserted into a vector through methods known in the art and described above. Also, the role of the elements in controlling gene expression can be confirmed by inserting the regulatory element into a construct containing a reporter gene and an appropriate cell as previously described.

The invention provides a construct containing a regulatory element that modulates the expression of a gene within a cell by posttranscriptional gene silencing. The regulatory element may be isolated according to the method of the invention as described in section IX of the detailed description. The construct contains the regulatory element inserted into a vector such that the regulatory element controls the expression of an open reading frame that is operably linked to the regulatory element. Methods to construct such a construct are well known in the art and are described herein (Sambrook et al., 1989). Thus, the expression of virtually any nucleic acid sequence placed under the control of the regulatory element in the construct may be regulated by posttranscriptional gene silencing. It is contemplated that the construct may be used to confer desired properties onto the cell of a plant that may be regulated by posttranscriptional gene silencing. Such properties include, but are not limited to, herbicide resistance, drought resistance or other properties known or described herein.

The invention also provides a method to block expression of endogenous genes of agricultural and other interest by transforming plants with sense constructs, particularly stem loop structures or inverted repeats to generate dsRNA. (Hamilton et al., Plant J., 15:737 (1998); Smith et al., Nature, 407:319 (2000); Chuang and Meyerowitz, Proc. Natl. Acad. Sci., USA, 97:4985 (2000); Schweizer et al., Plant J., 24:895 (2000)). This method may be used with constructs having regulatable promotors that are spread systemically. Accordingly, the method may be used with transgenic plants having inducable PTGS. Additionally, regulatable PTGS may be used to silence transcriptional repressors to activate expression of a target gene.

Cells may be produced that contain genomic mutations or deletions in genes corresponding to the polynucleotide sequences linked to both ends of the intervening sequence of the mutagenesis cassette as described. The method utilizes homologous recombination between the corresponding sequences to replace a section of a gene within the genome of a cell with the intervening sequence of the mutagenesis cassette. Methods for creating such mutations or deletions are well known in the art. See for example, Miao et al., Plant J., 7, 359 (1995); Rikkenink et al., Curr. Genet., 25: 202 (1994). Briefly, a mutagenesis cassette is introduced into a cell according to methods known in the art, such as through use of a A. tumefaciens T-DNA replacement vector. Finnemann et al., Plant Mol. Biol., 35: 523 (1997); Gallego et al., Plant Mol. Biol., 39: 83 (1999). Upon introduction into the cell, the mutagenesis cassette is inserted into the chromosome by the endogenous recombination system of the cell.

The invention provides a method to identify expression products that interact with expression products that are modulated within a cell by posttranscriptional gene silencing. In one embodiment a nucleic acid sequence encoding an expression product that is modulated is inserted into a vector for use in a yeast two-hybrid system. Such methods are well known in the art. Chien et al., P.N.A.S. (USA), 88:9578 (1991); Fields and Song, Nature, 340:245 (1989); Fields and Sternglanz, Trends Genet., 10:286 (1994). Briefly, in this method a protein is fused to a DNA-binding domain (the bait) and another protein is fused to a domain that activates RNA polymerase (here called the prey). These two constructs are expressed in two different haploid yeast strains of opposite mating type (MATa and MAT∀). The strains are mated to determine if the two proteins interact. Mating occurs when haploid yeast strains of opposite mating type come into contact, and results in fusion of the two haploids to form a diploid yeast strain. Thus, an interaction can be determined by measuring activation of a two-hybrid reporter gene in the diploid strain. The construct containing the nucleic acid sequence that encodes the interacting polypeptide may then be isolated and sequenced to determine the identity of the interacting polypeptide.

In another embodiment, an expression product that is modulated within a cell by posttranscriptional gene silencing is fused to a marker polypeptide. Marker polypeptides are well known in the art and include, but are not limited to, such polypeptides as antibody epitopes or glutathione S-transferase (GST). Kaelin, W. G. et al., Cell, 70:351 (1992). For example, a nucleic acid sequence encoding an expression product that is modulated within a cell by posttranscriptional gene silencing is inserted into a vector such that a fusion protein with GST is expressed. Such vectors are well known in the art and are commercially available. (Pharmacia, Piscataway, N.J.). The fusion protein formed may then be used to form a complex with other interacting proteins. This interaction may occur in vivo or in vitro. The complex may then be isolated through use of antibodies specific to the GST portion of the fusion protein. Such antibodies are commercially available. (Pharmacia, Piscataway, N.J.). Alternatively, the complex containing the GST fusion protein may be isolated through use of a glutathione agarose column (Sigma G-4510) or through other methods known in the art. The identity of the interacting expression product may then be determined by methods known in the art, such as for example, peptide sequencing.

The invention provides nucleic acid sequences that are modulated by PTGS that hybridize to nucleic acid segments corresponding to those listed in the sequence listing under low stringency conditions. Also provided are nucleic acid sequences that encode polypeptides that are substantially similar to those encoded by nucleic acid segments that correspond to those listed in the sequence listing. These orthologs may be determined through comparison of nucleic acid sequences that are modulated within a cell by PTGS to other sequences. The other sequences may be held in a database such as that maintained by the National Center for Biotechnology Information or other searchable databases. The comparison may be made visually or through methods well known in the art. Such methods include, for example, Blast searches and searches of the Swiss Protein Data Bank that are described herein. These orthologs may be used to transform cells and thereby confer onto the cells desired properties. Such properties include modulation of gene expression through use of PTGS. The orthologs may be introduced into many types of cells that include plant and animal cells. Cells and methods to introduce and express nucleic acids are contained herein. The transformed cells can be grown to produce transgenic plants and animals according to methods well known in the art and described herein. Accordingly, transgenic plants, animals, and other organisms can be propogated and used to produce products from the transgenic plants, animals and other organisms. Such products include seeds, fruits, progeny, products of the progeny, and the transgenic plants, animals, or other organisms themselves.

Also provided by the invention are methods to manipulate nucleic acid sequences that are modulated within a cell in response to contact of a virus with the cell. These sequences include variants, complements, and orthologs of those listed in the sequence listing. The following examples are for illustration only and are not meant to be limiting in any way.

An example of such manipulation is “gene shuffling” which may be used to prepare recombinant polypeptides having a particular activity. (See, for example, Crameri et al., Nature, 391, 288 (1998); Patten et al., Curr. Op. Biotech., 8, 724 (1997), U.S. Pat. Nos. 5,837,458; 5,834,252; 5,830,727; 5,811,238; 5,605,793; 6,132,970; 6,180,406). Briefly, a nucleic acid sequence may be fragmented and then religated to produce a polynucleic acid having an altered sequence. This altered nucleic acid may then be expressed and polypeptides encoded by the altered nucleic acid segment may be identified.

In another example, a nucleic acid sequence of the invention may be mutagenized to produce a polypeptide exhibiting altered activity. Such mutagenesis methods are well known in the art and are described herein. Further, a nucleic acid sequence of the invention may be mutagenized and shuffled to produce polypeptides having altered activities. See for example, U.S. Pat. No. 6,180,406.

The invention also provides a computer readable medium having stored thereon a data structure containing nucleic acid sequences that are modulated within a cell by PTGS. These sequences have at least 70% sequence identity to a nucleic acid sequence selected from those sequences corresponding to genes modulated within a cell by posttranscriptional gene silencing from cereals, but especially from rice, wheat and banana, identified in Tables 1 and 2 and listed in the sequence listing, as well as complementary, ortholog, and variant sequences thereof. Storage and use of nucleic acid sequences on a computer readable medium is well known in the art. (See for example U.S. Pat. Nos. 6,023,659; 5,867,402; 5,795,716) Examples of such medium include, but are not limited to, magnetic tape, optical disk, CD-ROM, random access memory, volitile memory, non-volitile memory and bubble memory. Accordingly, the nucleic acid sequences contained on the computer readable medium may be compared through use of a module that receives the sequence information and compares it to other sequence information. Examples of other sequences to which the nucleic acid sequences of the invention may be compared include those maintained by the National Center for Biotechnology Information (NCBI)(http://www.ncbi.nlm.nih.gov/) and the Swiss Protein Data Bank. A computer is an example of such a module that can read and compare nucleic acid sequence information. Accordingly, the invention also provides the method of comparing a nucleic acid sequence of the invention to another sequence. For example, a sequence of the invention may be submitted to the NCBI for a Blast search as described herein where the sequence is compared to sequence information contained within the NCBI database and a comparison is returned. The invention also provides nucleic acid sequence information in a computer readable medium that allows the encoded polypeptide to be optimized for a desired property. Examples of such properties include, but are not limited to, increased or decreased: thermal stability, chemical stability, hydrophylicity, hydrophobicity, and the like. Methods for the use of computers to model polypeptides and polynucleotides having altered activities are well known in the art and have been reviewed. (Lesyng B. and McCammon J A, Pharmocol. Ther., 60:149 (1993); Surles et al., Protein Sci., 3:198 (1994); Koehl P. and Delarue M., Curr. Opin. Struct. Biol., 6:222 (1996); Rossi et al., Biophys. J., 80:480 (2001)).

The polynucleic acid segments of the invention may be contained within a vector. A vector may include, but is not limited to, any plasmid, phagemid, F-factor, virus, cosmid, phage or Agrobacterium binary vector in double or single stranded linear or circular form which may or may not be self transmissible or mobilizable. The vector can also transform a prokaryotic or eukaryotic host either by integration into the cellular genome or exist extrachromosomally (e.g. autonomous replicating plasmid with an origin of replication).

Preferably the nucleic acid in the vector is under the control of, and operably linked to, an appropriate promoter or other regulatory elements for transcription in vitro or in a host cell such as a plant cell or microbe, e.g. bacteria. The vector may be a bi-functional expression vector which functions in multiple hosts. In the case of genomic DNA, this may contain its own promoter or other regulatory elements and in the case of cDNA this may be under the control of a promoter or other regulatory sequences for expression in a host cell.

Specifically included are shuttle vectors by which is meant a DNA vehicle capable, naturally or by design, of replication in two different host organisms, which may be selected from actinomycetes and related species, bacteria and eukaryotic cells (e.g. higher plant, mammalian, yeast or fungal).

The vector may also be a cloning vector which typically contain one or a small number of restriction endonuclease recognition sites at which foreign DNA sequences can be inserted in a determinable fashion. Such insertion can occur without loss of essential biological function of the cloning vector. A cloning vector may also contain a marker gene that is suitable for use in the identification and selection of cells transformed with the cloning vector. Examples of marker genes are tetracycline resistance, hygromycin resistance or ampicillin resistance. Many cloning vectors are commercially available (Stratagene, New England Biolabs, Clonetech).

The polynucleic acid segments of the invention may also be inserted into an expression vector. The selection of an appropriate expression vector will depend upon the method of introducing the expression vector into host cells. Typically an expression vector contains (1) prokaryotic DNA elements coding for a bacterial replication origin and an antibiotic resistance gene to provide for the amplification and selection of the expression vector in a bacterial host; (2) regulatory elements that control initiation of transcription such as a promoter; and (3) DNA elements that control the processing of transcripts such as introns, transcription termination/polyadenylation sequence; and (4) a reporter gene that is operatively linked to the DNA elements to control transcription initiation. Useful reporter genes include beta-glucuronidase, beta-galactosidase, chloramphenicol acetyl transferase, luciferase, green fluorescent protein (GFP) and the like. Preferably the reporter gene is either beta-glucuronidase (GUS), GFP or luciferase.

The general descriptions of plant expression vectors and reporter genes can be found in Gruber, et al., “Vectors for Plant Transformation, in Methods in Plant Molecular Biology & Biotechnology” in Glich et al., (Eds. pp. 89-119, CRC Press, 1993). Moreover GUS expression vectors and GUS gene cassettes are available from Clonetech Laboratories, Inc., Palo Alto, Calif. while luciferase expression vectors and luciferase gene cassettes are available from Promega Corp. (Madison, Wis.).

Methods to introduce a polynucleic acid segment into a vector are well known in the art (Sambrook et al., 1989).

The invention also provides an expression cassette which contains a DNA sequence capable of directing expression of a particular polynucleic acid segment of the invention either in vitro or in a host cell. Examples of such polynucleic acid segments are those segments corresponding to genes modulated within a cell by posttranscriptional gene silencing from cereals, but especially from rice, identified in Tables 1 and 2 and listed in the sequence listing or nucleic acid sequences having at least 70% nucleic acid identity to the sequences of in the sequence listing. Also, a polynucleic acid segment of the invention may be inserted into the expression cassette such that an anti-sense message is produced. The expression cassette is an isolatable unit such that the expression cassette may be in linear form and functional in in vitro transcription and translation assays. For example, the materials and procedures to conduct these assays are commercially available from Promega Corp. (Madison, Wis.). For example, an in vitro transcript may be produced by placing a polynucleotide sequence under the control of a T7 promoter and then using T7 RNA polymerase to produce an in vitro transcript. This transcript may then be translated in vitro through use of a rabbit reticulocyte lysate. Alternatively, the expression cassette can be incorporated into a vector allowing for replication and amplification of the expression cassette within a host cell or also in vitro transcription and translation of a polynucleotide sequence.

Such an expression cassette may contain one or a plurality of restriction sites allowing for placement of the polynucleic acid segment under the regulation of a regulatory sequence. The expression cassette can also contain a termination signal operably linked to the polynucleic acid segment as well as regulatory sequences required for proper translation of the polynucleic acid segment. The polynucleic acid segment may be one of the segments corresponding to genes modulated within a cell by posttranscriptional gene silencing from cereals, but especially from rice identified in Tables 1 and 2 and in the sequence listing, or a nucleic acid sequence having at least 70% sequence identity with any of in the sequence listing or a mutant thereof. The expression cassette containing the polynucleic acid segment may be chimeric, meaning that at least one of its components is heterologous with respect to at least one of its other components. The expression cassette may also be one which is naturally occurring but has been obtained in a recombinant form useful for heterologous expression. Expression of the polynucleic acid segment in the expression cassette may be under the control of a constitutive promoter or an inducible promoter which initiates transcription only when the host cell is exposed to some particular external stimulus.

The expression cassette may include in the 5′-3′ direction of transcription, a transcriptional and translational initiation region, a polynucleic acid segment and a transcriptional and translational termination region functional in vivo and/or in vitro. The termination region may be native with the transcriptional initiation region, may be native with the polynucleic acid segment, or may be derived from another source. Convenient termination regions are available from the Ti-plasmid of A. tumefaciens, such as the octopine synthase and nopaline synthase termination regions. See also, Guerineau et al., Mol. Gen. Genet., 262:141 (1991); Proudfoot, Cell, 64:671 (1991); Sanfacon et al., Genes Dev., 5:141 (1991); Mogen et al., Plant Cell, 2:1261 (1990); Munroe et al., Gene, 91:151 (1990); Ballas et al., Nucleic Acids Res., 17:7891 (1989); Joshi et al., Nucleic Acid Res., 15:9627 (1987).

The regulatory sequence can be a polynucleotide sequence located upstream (5′ non-coding sequences), within, or downstream (3′ non-coding sequences) of a coding sequence, and which influences the transcription, RNA processing or stability, or translation of the associated coding sequence. Regulatory sequences can include, but are not limited to, enhancers, promoters, repressor binding sites, translation leader sequences, introns, and polyadenylation signal sequences. They may include natural and synthetic sequences as well as sequences which may be a combination of synthetic and natural sequences. While regulatory sequences are not limited to promoters, some useful regulatory sequences include constitutive promoters, inducible promoters, regulated promoters, tissue-specific promoters, viral promoters and synthetic promoters.

A promoter is a nucleotide sequence which controls the expression of the coding sequence by providing the recognition for RNA polymerase and other factors required for proper transcription. A promoter includes a minimal promoter, consisting only of all basal elements needed for transcription initiation, such as a TATA-box and/or initiator that is a short DNA sequence comprised of a TATA-box and other sequences that serve to specify the site of transcription initiation, to which regulatory elements are added for control of expression. A promoter may be derived entirely from a native gene, or be composed of different elements derived from different promoters found in nature, or even be comprised of synthetic DNA segments. A promoter may contain DNA sequences that are involved in the binding of protein factors which control the effectiveness of transcription initiation in response to physiological or developmental conditions. A promoter may also include a minimal promoter plus a regulatory element or elements that are capable of controlling the expression of a coding sequence or functional RNA. This type of promoter sequence consists of proximal and more distal elements, the latter elements are often referred to as enhancers. The promoter may also be inducible.

Several inducible promoters have been reported. Many are described in a review by Gatz (Current Opinion in Biotechnology, 7:168 (1996); Gatz, C., Annu. Rev. Plant Physiol. Plant Mol. Biol., 48:89 (1997)). Examples include tetracycline repressor system, Lac repressor system, copper-inducible systems, salicylate-inducible systems (such as the PR1a system), glucocorticoid-(Aoyama T. et al., N-H Plant Journal, 11:605 (1997)) and ecdysome-inducible systems. Also included are the benzene sulphonamide-(U.S. Pat. No. 5,364,780) and alcohol-(WO 97/06269 and WO 97/06268) inducible systems and glutathione S-transferase promoters. In the case of a multicellular organism, the promoter can also be specific to a particular tissue or organ or stage of development.

An enhancer is a DNA sequence which can stimulate promoter activity and may be an innate element of the promoter or a heterologous element inserted to enhance the level or tissue specificity of a promoter. It is capable of operating in both orientations (normal or flipped), and is capable of functioning even when moved either upstream or downstream from the promoter. Both enhancers and other upstream promoter elements bind sequence-specific DNA-binding proteins that mediate their effects.

The expression cassette can contain a 5′ non-coding sequence which is a nucleotide sequence located 5′ (upstream) to the coding sequence. It is present in the fully processed mRNA upstream of the initiation codon and may affect processing of the primary transcript to mRNA, stability of the mRNA or translation efficiency. Turner et al., Molecular Biotechnology, 3:225 (1995).

The expression cassette may also contain a 3′ non-coding sequence which is a nucleotide sequence located 3′(downstream) to a coding sequence and includes polyadenylation signal sequences and other sequences encoding regulatory signals capable of affecting mRNA processing or gene expression. The polyadenylation signal is usually characterized by affecting the addition of polyadenylic acid tracts to the 3′ end of the mRNA precursor. The use of different 3′ non-coding sequences is exemplified by Ingelbrecht et al., Plant Cell, 1:671 (1989).

The invention also provides a construct containing a vector and an expression cassette. The vector may be selected from, but not limited to, any vector described herein. Into this vector may be inserted an expression cassette containing the nucleic acid sequences of the invention through methods known in the art and previously described (Sambrook et al., 1989). In one embodiment, the regulatory sequences of the expression cassette may be derived from a source other than the vector into which the expression cassette is inserted. In another embodiment, a construct containing a vector and an expression cassette is formed upon insertion of a polynucleic acid segment of the invention into a vector that itself contains regulatory sequences. Thus, an expression cassette is formed upon insertion of the polynucleic acid segment into the vector. Vectors containing regulatory sequences are available commercially and methods for their use are known in the art (Clonetech, Promega, Stratagene).

The invention also provides a mutagenesis cassette. In one embodiment, the mutagenesis cassette contains an intervening nucleotide sequence linked on both ends to a flanking nucleotide sequence that hybridizes under low stringency conditions to a gene that is modulated within a cell by posttranscriptional gene silencing. Preferably the flanking nucleotide sequence is a sequence having at least 70% nucleic acid sequence identity to a nucleic acid sequence corresponding to genes modulated within a cell by posttranscriptional gene silencing from cereals, but especially from rice, identified in Tables 1 and 2 and described by any of the sequences in the sequence listing or the complement, variant, or ortholog thereof. More preferably the flanking nucleotide sequence is a sequence described by any of in the sequence listing or the complement thereof. The intervening sequence may be any sequence that is of a length allowing it to be recombined into the DNA of a cell. Preferably the intervening sequence encodes a mutant of a gene that is modulated within a cell by posttranscriptional gene silencing. More preferably the intervening sequence encodes a mutant of a polynucleic acid segment corresponding to genes modulated within a cell by posttranscriptional gene silencing from cereals, but especially from rice, identified in Tables 1 and 2 and described by one of the sequences in the sequence listing. The intervening sequence may also encode a selectable marker. More preferably the intervening sequence confers chemical resistance.

The mutagenesis cassette may be constructed through methods known in the art (Sambrook et al., 1989). In one example put forth for illustration only and not meant to be limiting in any way, a polynucleic acid segment as described by in the sequence listing can be inserted into a vector through use of one or more restriction endonucleases and ligase as previously described. Next, one or more restriction endonuclease recognition sites are chosen that are within the inserted sequence. The inserted sequence is digested with one or more endonucleases to create a linearized nucleic acid sequence having the vector sequence linked on both ends to fragments of the inserted sequence. A nucleic acid sequence that is to be the intervening sequence of the mutagenesis cassette is then digested with restriction endonucleases to produce a polynucleic acid fragment having ends able to be ligated to the ends of the inserted sequence that are linked to the vector sequence. The ends of the intervening sequence are then ligated to the ends of the inserted sequence. This forms a circularized construct having an intervening sequence linked on both sides to fragments of the polynucleic acid segment corresponding to genes modulated within a cell by posttranscriptional gene silencing from cereals, but especially from rice, identified in Tables 1 and 2 and described by any of the sequences in the sequence listing. The construct may be further digested with restriction endonucleases that recognize sites within the vector sequence to produce a linear cassette containing the intervening sequence linked on both ends to a polynucleic acid segment of in the sequence listing. The mutagenesis cassette of the invention may also be incorporated into a vector. This may be done according to methods known in the art or as previously described (Sambrook et al., 1989). Preferably the vector is a plasmid. More preferably the vector is the Ti plasmid.

The expression and/or mutagenesis cassette according to the invention, or a vector construct containing the expression and/or mutagenesis cassette may be inserted into a cell. The expression cassette or vector construct may be carried episomally or integrated into the genome of the cell. The transformed cell may then be grown into a transgenic organism, such as a plant or animal. Accordingly, the invention provides the products of the transgenic plant, animal, or other organism. Such products may include, but are not limited to, seeds, fruits, progeny, products of the progeny, and the transgenic plant, animal or other organism.

A variety of techniques are available and known to those skilled in the art for introduction of constructs including vectors, expression cassettes, expression vectors, etc, into a cellular host. Transformation of bacteria and many eukaryotic cells may be accomplished through use of polyethylene glycol, calcium chloride, viral infection, phage infection, electroporation and other methods known in the art.

Techniques for transforming plant cells include transformation with DNA employing A. tumefaciens or A. rhizogenes as the transforming agent, electroporation, DNA injection, microprojectile bombardment, particle acceleration, etc. (See, for example, EP 295959 and EP 138341).

Many vectors are available for transformation using Agrobacterium tumefaciens. It is particularly preferred to use the binary type vectors of Ti and Ri plasmids of Agrobacterium spp. such as pBIN19. Bevan, Nucl. Acids Res. (1984). An additional vector useful for Agrobacterium-mediated transformation is the binary vector pCIB 10, which contains a gene encoding kanamycin resistance for selection in plants, T-DNA right and left border sequences and incorporates sequences from the wide host- range plasmid pRK252 allowing it to replicate in both E. coli and Agrobacterium. Its construction is described by Rothstein et al., Gene, 53, 153 (1987). Various derivatives of pCIB10 have been constructed which incorporate the gene for hygromycin B phosphotransferase described by Gritz et al., Gene, 25, 179 (1983). These derivatives enable selection of transgenic plant cells on hygromycin only (pCIB743), or hygromycin and kanamycin (pCIB715, pCIB717).

Ti-derived vectors transform a wide variety of higher plants, including monocotyledonous and dicotyledonous plants, such as soybean, cotton, rape, tobacco, and rice (Pacciotti et al. Bio/Technology, 3:241 (1985): Byrne et al. Plant Cell Tissue and Organ Culture, 8:3 (1987); Sukhapinda et al. Plant Mol. Biol., 8:209 (1987); Lorz et al. Mol. Gen. Genet., 199:178 (1985); Potrykus Mol. Gen. Genet., 199:183 (1985); Park et al., J. Plant Biol., 38:365 (1985): Hiei et al., i Plant J., 6:271(1994). The use of T-DNA to transform plant cells has received extensive study and is amply described (EP 120516; Hoekema, In: The Binary Plant Vector System. Offset-drukkerij Kanters B. V.; Alblasserdam (1985), Chapter V; Knauf, et al., Genetic Analysis of Host Range Expression by Agrobacterium In: Molecular Genetics of the Bacteria-Plant Interaction, Puhler, A. ed., Springer-Verlag, New York, 1983, p. 245; and An. et al., EMBO J., 4:277 (1985).

The expression cassettes and vectors of the present invention can be introduced into the plant cell in a number of art-recognized ways. Those skilled in the art will appreciate that the choice of method might depend on the type of plant, i.e., monocotyledonous or dicotyledonous, targeted for transformation. Suitable methods of transforming plant cells include, but are not limited to, microinjection (Crossway et al., BioTechniques, 4, 320 (1986)), electroporation (Riggs et al., Proc. Natl. Acad. Sci. USA, 83, 5602 (1986), Agrobacterium-mediated transformation (De Blaere et al., Meth. Enzymol., 143, 277 (1987); Hinchee et al., Biotechnology, 6, 915 (1988)), direct gene transfer (Paszkowski et al., EMBO J., 3, 2717 (1984)), and ballistic particle acceleration using devices available from Agracetus, Inc., Madison, Wis. and BioRad, Hercules, Calif. (see, for example, Sanford et al., U.S. Pat. No. 4,945,050; and McCabe et al., Biotechnology, 6, 923-926 (1988)). Also see, Weissinger et al., Annual Rev. Genet., 22, 421 (1988); Sanford et al., Particulate Science and Technology, 5, 27 (1987)(onion); Christou et al., Plant Physiol., 87, 671 (1988)(soybean); McCabe et al., Bio/Technology, 6, 923 (1988)(soybean); Datta et al., Bio/Technology, 8, 736 (1990)(rice); Klein et al., Proc. Natl. Acad. Sci. USA, 85, 4305 (1988)(maize); Klein et al., Bio/Technology, 6, 559 (1988)(maize); Klein et al., Plant Physiol., 91:440 (1988)(maize); Fromm et al., Bio/Technology, 8, 833 (1990)(maize); and Gordon-Kamm et al., Plant Cell, 2, 603 (1990)(maize); Svab et al., Proc. Natl. Acad. Sci. USA, 87, 8526 (1990)(tobacco chloroplast); Koziel et al., Biotechnology, 11, 194 (1993)(maize); Shimamoto et al., Nature, 338, 274 (1989)(rice); Christou et al., Biotechnology, 9, 957 (1991)(rice); European Patent Application EP 0 332 581 (orchardgrass and other Pooideae); Vasil et al., Biotechnology, 11, 1553 (1993)(wheat); Weeks et al., Plant Physiol., 102, 1077 (1993)(wheat). Methods in Moleculary Biology, 82. Arabidopsis Protocols Ed. Martinez-Zapater and Salinas 1998 Humana Press (Arabidopsis).

Transformation of plants can be undertaken with a single DNA molecule or multiple DNA molecules (i.e., co-transformation), and both these techniques are suitable for use with the expression cassettes of the present invention. Numerous transformation vectors are available for plant transformation, and the expression cassettes of this invention can be used in conjunction with any such vectors. The selection of vector will depend upon the preferred transformation technique and the target species for transformation.

For certain plant species, different antibiotic or herbicide selection markers may be preferred. Selection markers used routinely in transformation include the nptII gene which confers resistance to kanamycin and related antibiotics (Messing & Vierra, Gene, 19: 259 (1982); Bevan et al., Nature, 304: 184 (1983)), the bar gene which confers resistance to the herbicide phosphinothricin (White et al., Nucl Acids Res, 18; 1062 (1990), Spencer et al., Theor. Appl. Genet, 79: 625 (1990)), the hph gene which confers resistance to the antibiotic hygromycin (Blochinger & Diggelmann, Mol Cell Biol, 4: 2929), and the dhfr gene, which confers resistance to methotrexate (Bourouis et al., EMBO J., 2;1099 (1983)).

One such vector useful for direct gene transfer techniques in combination with selection by the herbicide Basta (or phosphinothricin) is pCIB3064. This vector is based on the plasmid pCIB246, which comprises the CaMV 35S promoter in operational fusion to the E. coli GUS gene and the CaMV 35S transcriptional terminator and is described in the PCT published application WO 93/07278, herein incorporated by reference. One gene useful for conferring resistance to phosphinothricin is the bar gene from Streptomyces viridochromogenes (Thompson et al., EMBO J, 6: 2519 (1987)). This vector is suitable for the cloning of plant expression cassettes containing their own regulatory signals. An additional transformation vector is pSOG35 which utilizes the E. coli gene dihydrofolate reductase (DHFR) as a selectable marker conferring resistance to methotrexate.

Plant species may be transformed with a construct of the present invention by the DNA-mediated transformation of plant cell protoplasts and subsequent regeneration of the plant from the transformed protoplasts in accordance with procedures well known in the art.

Any plant tissue capable of subsequent clonal propagation, whether by organogenesis or embryogenesis, may be transformed with a construct of the present invention. The term organogenesis means a process by which shoots and roots are developed sequentially from meristematic centers while the term embryogenesis means a process by which shoots and roots develop together in a concerted fashion (not sequentially), whether from somatic cells or gametes. The particular tissue chosen will vary depending on the clonal propagation systems available for, and best suited to, the particular species being transformed. Exemplary tissue targets include leaf disks, pollen, embryos, cotyledons, hypocotyls, megagametophytes, callus tissue, existing meristematic tissue (e.g., apical meristems, axillary buds, and root meristems), and induced meristem tissue (e.g., cotyledon meristem and hypocotyl meristem).

Plants of the present invention may take a variety of forms. The plants may be chimeras of transformed cells and non-transformed cells; the plants may be clonal transformants (e.g., all cells transformed to contain the expression cassette); the plants may comprise grafts of transformed and untransformed tissues (e.g., a transformed root stock grafted to an untransformed scion in citrus species). The transformed plants may be propagated by a variety of means, such as by clonal propagation or classical breeding techniques. For example, first generation (or T1) transformed plants may be selfed to give homozygous second generation (or T2) transformed plants, and the T2 plants further propagated through classical breeding techniques. A dominant selectable marker (such as npt II) can be associated with the expression cassette to assist in breeding.

The present invention may be used for transformation of any plant species, including, but not limited to, corn (Zea mays), Brassica sp. (e.g., B. napus, B. rapa, B. juncea), particularly those Brassica species useful as sources of seed oil, alfalfa (Medicago sativa), rice (Oryza sativa), rye (Secale cereale), sorghum (Sorghum bicolor, Sorghum vulgare), millet (e.g., pearl millet (Pennisetum glaucum), proso millet (Panicum miliaceum), foxtail millet (Setaria italica), finger millet (Eleusine coracana)), sunflower (Helianthus annuus), safflower (Carthamus tinctorius), wheat (Triticum aestivum), soybean (Glycine max), tobacco (Nicotiana tabacum), potato (Solanum tuberosum), peanuts (Arachis hypogaea), cotton (Gossypium barbadense, Gossypium hirsutum), sweet potato (Ipomoea batatus), cassava (Manihot esculenta), coffee (Cofea spp.), coconut (Cocos nucifera), pineapple (Ananas comosus), citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), olive (Olea europaea), papaya (Carica papaya), cashew (Anacardium occidentale), macadamia (Macadamia integrifolia), almond (Prunus amygdalus), sugar beets (Beta vulgaris), sugarcane (Saccharum spp.), oats, barley, vegetables, ornamentals, and conifers.

Vegetables include tomatoes (Lycopersicon esculentum), lettuce (e.g., Lactuca sativa), green beans (Phaseolus vulgaris), lima beans (Phaseolus limensis), peas (Lathyrus spp.), and members of the genus Cucumis such as cucumber (C. sativus), cantaloupe (C. cantalupensis), and musk melon (C. melo). Ornamentals include azalea (Rhododendron spp.), hydrangea (Macrophylla hydrangea), hibiscus (Hibiscus rosasanensis), roses (Rosa spp.), tulips (Tulipa spp.), daffodils (Narcissus spp.), petunias (Petunia hybrida), carnation (Dianthus caryophyllus), poinsettia (Euphorbia pulcherrima), and chrysanthemum. Conifers that may be employed in practicing the present invention include, for example, pines such as loblolly pine (Pinus taeda), slash pine (Pinus elliotii), ponderosa pine (Pinus ponderosa), lodgepole pine (Pinus contorta), and Monterey pine (Pinus radiata), Douglas-fir (Pseudotsuga menziesii); Western hemlock (Tsuga canadensis); Sitka spruce (Picea glauca); redwood (Sequoia sempervirens); true firs such as silver fir (Abies amabilis) and balsam fir (Abies balsamea); and cedars such as Western red cedar (Thuja plicata) and Alaska yellow-cedar (Chamaecyparis nootkatensis). Leguminous plants include beans and peas. Beans include guar, locust bean, fenugreek, soybean, garden beans, cowpea, mungbean, lima bean, fava bean, lentils, chickpea, etc. Legumes include, but are not limited to, Arachis, e.g., peanuts, Vicia, e.g., crown vetch, hairy vetch, adzuki bean, mung bean, and chickpea, Lupinus, e.g., lupine, trifolium, Phaseolus, e.g., common bean and lima bean, Pisum, e.g., field bean, Melilotus, e.g., clover, Medicago, e.g., alfalfa, Lotus, e.g., trefoil, lens, e.g., lentil, and false indigo. Preferred forage and turf grass for use in the methods of the invention include alfalfa, orchard grass, tall fescue, perennial ryegrass, creeping bent grass, and redtop.

Preferably, plants of the present invention are crop plants (for example, corn, alfalfa, sunflower, Brassica, soybean, cotton, safflower, peanut, sorghum, wheat, millet, tobacco, barley, rice, tomato, potato, squash, melons, legume crops, etc.).

Transgenic plant cells are then placed in an appropriate selective medium for selection of transgenic cells which are then grown to callus. Shoots are grown from callus and plantlets generated from the shoot by growing in rooting medium. The various constructs normally will be joined to a marker for selection in plant cells. Conveniently, the marker may be resistance to a biocide (particularly an antibiotic, such as kanamycin, G418, bleomycin, hygromycin, chloramphenicol, herbicide, or the like). The particular marker used will allow for selection of transformed cells as compared to cells lacking the DNA which has been introduced. Components of DNA constructs, including transcription/expression cassettes of this invention, may be prepared from sequences which are native (endogenous) or foreign (exogenous) to the host. By “foreign” it is meant that the sequence is not found in the wild-type host into which the construct is introduced. Heterologous constructs will contain at least one region which is not native to the gene from which the transcription-initiation-region is derived.

To confirm the presence of the transgenes in transgenic cells and plants, a Southern blot analysis can be performed using methods known to those skilled in the art. Integration of a polynucleic acid segment into the genome can be detected and quantitated by Southern blot, since they can be readily distinguished from constructs containing the segments through use of appropriate restriction enzymes. Expression products of the transgenes can be detected in any of a variety of ways, depending upon the nature of the product, and include Western blot and enzyme assay. One particularly useful way to quantitate protein expression and to detect replication in different plant tissues is to use a reporter gene, such as GUS. Once transgenic plants have been obtained, they may be grown to produce plant tissues or parts having the desired phenotype. The plant tissue or plant parts may be harvested, and/or the seed collected. The seed may serve as a source for growing additional plants with tissues or parts having the desired characteristics.

The present invention also provides for the production of transgenic non-human animal models in which post-transcriptional gene silencing is present, or in which post-transcriptional gene silencing has been inactivated (e.g., “knock-out” deletions). Animal species suitable for use in the animal models of the present invention include, but are not limited to, rats, mice, hamsters, guinea pigs, rabbits, dogs, cats, goats, sheep, pigs, and nonhuman primates (e.g., Rhesus monkeys, chimpanzees). For initial studies, transgenic rodents (e.g., mice) are preferred due to their relative ease of maintenance. Indeed, as noted above, transgenic yeast or invertebrates (e.g., nematodes, insects) may be preferred for some studies because they will allow for even more rapid and inexpensive screening.

To create an animal model (e.g., a transgenic mouse), a normal or mutant polynucleic acid segement corresponding the sequence identifiers numbered 1-145, or the complement, ortholog, or variant thereof, can be inserted into a germ line or stem cell using standard techniques of oocyte microinjection, or transfection or microinjection into embryonic stem cells. Animals produced by these or similar processes are referred to as transgenic. Similarly, if it is desired to inactivate or replace an endogenous gene, homologous recombination using embryonic stem cells may be employed. Animals produced by these or similar processes are referred to as “knock-out” (inactivation) or “knock-in” (replacement) models.

For oocyte injection, one or more copies of the recombinant DNA constructs of the present invention may be inserted into the pronucleus of a just-fertilized oocyte. This oocyte is then reimplanted into a pseudo-pregnant foster mother. The liveborn animals are screened for integrants using analysis of DNA (e.g., from the tail veins of offspring mice) for the presence of the inserted recombinant transgene sequences. The transgene may be either a complete genomic sequence injected as a YAC, BAC, PAC or other chromosome DNA fragment, a cDNA with either the natural promoter or a heterologous promoter, or a minigene containing all of the coding region and other elements found to be necessary for optimum expression.

Retroviral infection of early embryos can also be done to insert the recombinant DNA constructs of the invention. In this method, the transgene is inserted into a retroviral vector which is used to infect embryos (e.g., mouse or non-human primate embryos) directly during the early stages of development to generate chimeras, some of which will lead to germline transmission.

Homologous recombination using stem cells allows for the screening of gene transfer cells to identify the rare homologous recombination events. Once identified, these can be used to generate chimeras by injection of blastocysts, and a proportion of the resulting animals will show germline transmission from the recombinant line. This methodology is especially useful if inactivation of a gene is desired. Homologous recombination leads to the insertion of the marker sequences in the middle of an exon, causing inactivation of the target gene and/or deletion of internal sequences. DNA analysis of individual clones can then be used to recognize the homologous recombination events.

The techniques of generating transgenic animals, as well as the techniques for homologous recombination or gene targeting, are now widely accepted and practiced. A laboratory manual on the manipulation of the mouse embryo, for example, is available detailing standard laboratory techniques for the production of transgenic mice (Hogan et al. (1986) Manipulating the Mouse Embryo, Cold Spring Harbor Laboratory Press, Cold Spring Harbor, N.Y.). To create a transgene, the target sequence of interest is typically ligated into a cloning site located downstream of some promoter element which will regulate the expression of RNA from the sequence. Downstream of the coding sequence, there is typically an artificial polyadenylation sequence. An alternate approach to creating a transgene is to use an endogenous promoter and regulatory sequences to drive expression of the transgene. Finally, it is possible to create transgenes using large genomic DNA fragments such as YACs which contain the entire desired gene as well as its appropriate regulatory sequences. (Lamb et al.. Nature Genetics, 5:22 (1993)).

Within a plant promoter region there are several domains that are necessary for full function of the promoter. The first of these domains lies immediately upstream of the structural gene and forms the “core promoter region” containing consensus sequences, normally 70 base pairs immediately upstream of the gene. The core promoter region contains the characteristic CAAT and TATA boxes plus surrounding sequences, and represents a transcription initiation sequence that defines the transcription start point for the structural gene.

The presence of the core promoter region defines a sequence as being a promoter: if the region is absent, the promoter is non-functional. Furthermore, the core promoter region is insufficient to provide full promoter activity. A series of regulatory sequences upstream of the core constitute the remainder of the promoter. The regulatory sequences determine expression level, the spatial and temporal pattern of expression and, for an important subset of promoters, expression under inductive conditions (regulation by external factors such as light, temperature, chemicals, hormones).

A range of naturally-occurring promoters are known to be operative in plants and have been used to drive the expression of heterologous (both foreign and endogenous) genes in plants: for example, the constitutive 35S cauliflower mosaic virus (CaMV) promoter, the ripening-enhanced tomato polygalacturonase promoter (Bird et al., Plant Molecular Biology, 11:651 (1988)), the E8 promoter (Diekman & Fischer, EMBO, 7:3315 (1988)) and the fruit specific 2A1 promoter (Pear et al., Plant Molecular Biology, 13:639 (1989)) and many others.

Two principal methods for the control of expression are known, viz.: overexpression and underexpression. Overexpression can be achieved by insertion of one or more than one extra copy of the selected gene. It is, however, not unknown for plants or their progeny, originally transformed with one or more than one extra copy of a nucleotide sequence, to exhibit the effects of underexpression as well as overexpression. For underexpression there are two principle methods which are commonly referred to in the art as “antisense downregulation” and “sense downregulation” (sense downregulation is also referred to as “cosuppression”). Generically these processes are referred to as “gene silencing”Both of these methods lead to an inhibition of expression of the target gene.

Obtaining sufficient levels of transgene expression in the appropriate plant tissues is an important aspect in the production of genetically engineered crops. Expression of heterologous DNA sequences in a plant host is dependent upon the presence of an operably linked promoter that is functional within the plant host. Choice of the promoter sequence will determine when and where within the organism the heterologous DNA sequence is expressed.

Therefore, the selection of promoters for directing expression of a given transgene is critical. Promoters which are useful for plant transgene expression include those that are inducible, viral, synthetic, constitutive (Poszkowski et al., 1989; Odell et al., Nature, 313:810 (1985)), temporally regulated, spatially regulated, tissue-specific, and spatio-temporally regulated (Chau et al., 1989).

Where expression in specific tissues or organs is desired, tissue-specific promoters may be used. In contrast, where gene expression in response to a stimulus is desired, inducible promoters are the regulatory elements of choice. Where continuous expression is desired throughout the cells of a plant, constitutive promoters are utilized. Additional regulatory sequences upstream and/or downstream from the core promoter sequence may be included in expression constructs of transformation vectors to bring about varying levels of expression of heterologous nucleotide sequences in a transgenic plant.

A number of plant promoters have been described with various expression characteristics. Examples of some constitutive promoters which have been described include the rice actin 1 (Wang et al., Mol. Cell. Biol., 12:3399 (1992); U.S. Pat. No. 5,641,876), CaMV 35S (Odell et al., Nature, 313:810 (1985)), CaMV 19S (Lawton et al., 1987), nos (Ebert et al., 1987), Adh (Walker et al., 1987), sucrose synthase (Yang & Russell, 1990); and the ubiquitin promoters.

Examples of tissue specific promoters which have been described include the lectin (Vodkin, Prog. Clin. Biol. Res., 138;87 (1983); Lindstrom et al., Der. Genet., 11:160 (1990),) corn alcohol dehydrogenase 1 (Vogel et al., 1989; Dennis et al., Nucleic Acids Res., 12:3983 (1984)), corn light harvesting complex (Simpson, 1986; Bansal et al., Proc. Natl. Acad. Sci. USA, 89:3654 (1992)), corn heat shock protein (Odell et al., 1985; Rochester et al., 1986), pea small subunit RuBP carboxylase (Poulsen et al., 1986; Cashmore et al., 1983), Ti plasmid mannopine synthase (Langridge et al., 1989), Ti plasmid nopaline synthase (Langridge et al., 1989), petunia chalcone isomerase (vanTunen et al., EMBO J., 7;1257(1988)), bean glycine rich protein 1 (Keller et al., Genes Dev., 3:1639 (1989)), truncated CaMV 35s (Odell et al., Nature, 313:810 (1985)), potato patatin (Wenzler et al., Plant Mol. Biol., 13:347 (1989)), root cell (Yamamoto et al., Nucleic Acids Res., 18:7449 (1990)), maize zein (Reina et al., Nucleic Acids Res., 18:6425 (1990); Kriz et al., Mol. Gen. Genet., 207:90 (1987); Wandelt et al., Nucleic Acids Res., 17:2354 (1989); Langridge et al., Cell, 34:1015 (1983); Reina et al., Nucleic Acids Res., 18:7449 (1990)), globulin-I (Belanger et al., Genetics, 129:863 (1991)), α-tubulin, cab (Sullivan et al., Mol. Gen. Genet., 215:431 (1989)), PEPCase (Hudspeth & Grula, 1989), R gene complex-associated promoters (Chandler et al., Plant Cell, 1:1175 (1989)), and chalcone synthase promoters (Franken et al., EMBO J., 10:2605 (1991)).

Inducible promoters that have been described include the ABA- and turgor-inducible promoters, the promoter of the auxin-binding protein gene (Schwob et al., Plant J., 4:423 (1993)), the UDP glucose flavonoid glycosyl-transferase gene promoter (Ralston et al., Genetics, 119:185 (1988)), the MPI proteinase inhibitor promoter (Cordero et al., Plant J., 6:141 (1994)), and the glyceraldehyde-3-phosphate dehydrogenase gene promoter (Kohler et al., Plant Mol. Biol., 29;1293 (1995); Quigley et al., J. Mol. Evol., 29:412 (1989); Martinez et al., J. Mol. Biol., 208:551 (1989)).

Several tissue-specific regulated genes and/or promoters have been reported in plants. These include genes encoding the seed storage proteins (such as napin, cruciferin, beta-conglycinin, and phaseolin) zein or oil body proteins (such as oleosin), or genes involved in fatty acid biosynthesis (including acyl carrier protein, stearoyl-ACP desaturase. And fatty acid desaturases (fad 2-1)), and other genes expressed during embryo development (such as Bce4, see, for example. EP 255378 and Kridl et al., Seed Science Research, 1:209 (1991)). Particularly useful for seed-specific expression is the pea vicilin promoter (Czako et al., Mol. Gen. Genet., 235:33 (1992). (See also U.S. Pat. No. 5,625,136, herein incorporated by reference.) Other useful promoters for expression in mature leaves are those that are switched on at the onset of senescence, such as the SAG promoter from Arabidopsis (Gan et al., Science, 270:1986 (1995).

A class of fruit-specific promoters expressed at or during antithesis through fruit development, at least until the beginning of ripening, is discussed in U.S. Pat. No. 4,943,674, the disclosure of which is hereby incorporated by reference. cDNA clones that are preferentially expressed in cotton fiber have been isolated (John et al., Proc. Natl. Acad. Sci. USA, 89:5769 (1992). cDNA clones from tomato displaying differential expression during fruit development have been isolated and characterized (Mansson et al., Gen. Genet., 200:356 (1985), Slater et al., Plant Mol. Biol., 5:137 (1985)). The promoter for polygalacturonase gene is active in fruit ripening. The polygalacturonase gene is described in U.S. Pat. No. 4,535,060, U.S. Pat. No. 4,769,061, U.S. Pat. No. 4,801,590, and U.S. Pat. No. 5,107,065, which disclosures are incorporated herein by reference.

Other examples of tissue-specific promoters include those that direct expression in leaf cells following damage to the leaf (for example, from chewing insects), in tubers (for example, patatin gene promoter), and in fiber cells (an example of a developmentally-regulated fiber cell protein is E6 (John et al., Proc. Natl. Acad. Sci. USA, 89:5769 (1992). The E6 gene is most active in fiber, although low levels of transcripts are found in leaf, ovule and flower.

The tissue-specificity of some “tissue-specific” promoters may not be absolute and may be tested by one skilled in the art using the diphtheria toxin sequence. One can also achieve tissue-specific expression with “leaky” expression by a combination of different tissue-specific promoters (Beals et al., Plant Cell, 9:1527 (1997)). Other tissue-specific promoters can be isolated by one skilled in the art (see U.S. 5,589,379). Several inducible promoters (“gene switches”) have been reported. Many are described in the review by Gatz (Current Opinion in Biotechnology, 7:168 (1996); Gatz, C., Annu. Rev. Plant Physiol. Plant Mol. Biol., 48:89 (1997)). These include tetracycline repressor system, Lac repressor system, copper-inducible systems, salicylate-inducible systems (such as the PR1a system), glucocorticoid-(Aoyama T. et al., N-H Plant Journal, 11:605 (1997)) and ecdysome-inducible systems. Also included are the benzene sulphonamide-(U.S. Pat. No. 5364,780) and alcohol-(WO 97/06269 and WO 97/06268) inducible systems and glutathione S-transferase promoters. Other studies have focused on genes inducibly regulated in response to environmental stress or stimuli such as increased salinity. Drought, pathogen and wounding. (Graham et al., J. Biol. Chem., 260:6555 (1985); Graham et al., J. Biol. Chem., 260:6561 (1985), Smith et al., Planta, 168:94 (1986)). Accumulation of metallocarboxypeptidase-inhibitor protein has been reported in leaves of wounded potato plants (Graham et al., Biochem. Biophys. Res. Comm., 101:1164 (1981)). Other plant genes have been reported to be induced methyl jasmonate, elicitors, heat-shock, anaerobic stress, or herbicide safeners.

Regulated expression of the chimeric transacting viral replication protein can be further regulated by other genetic strategies. For example, Cre-mediated gene activation as described by Odell et al. Mol. Gen. Genet., 113:369 (1990). Thus, a DNA fragment containing 3′ regulatory sequence bound by lox sites between the promoter and the replication protein coding sequence that blocks the expression of a chimeric replication gene from the promoter can be removed by Cre-mediated excision and result in the expression of the trans-acting replication gene. In this case, the chimeric Cre gene, the chimeric trans-acting replication gene, or both can be under the control of tissue-and developmental-specific or inducible promoters. An alternate genetic strategy is the use of tRNA suppressor gene. For example, the regulated expression of a tRNA suppressor gene can conditionally control expression of a trans-acting replication protein coding sequence containing an appropriate termination codon as described by Ulmasov et al. Plant Mol. Biol., 35:417 (1997). Again, either the chimeric tRNA suppressor gene, the chimeric transacting replication gene, or both can be under the control of tissue- and developmental-specific or inducible promoters.

Frequently it is desirable to have continuous or inducible expression of a DNA sequence throughout the cells of an organism in a tissue-independent manner. For example, increased resistance of a plant to infection by soil- and air borne pathogens might be accomplished by genetic manipulation of the plant's genome to comprise a continuous promoter operably linked to a heterologous pathogen-resistance gene such that pathogen-resistance proteins are continuously expressed throughout the plant's tissues.

Alternatively, it might be desirable to inhibit expression of a native DNA sequence within a plant's tissues to achieve a desired phenotype. In this case, such inhibition might be accomplished with transformation of the plant to comprise a constitutive, tissue-independent promoter operably linked to an antisense nucleotide sequence, such that constitutive expression of the antisense sequence produces an RNA transcript that interferes with translation of the mRNA of the native DNA sequence.

To define a minimal promoter region, a DNA segment representing the promoter region is removed from the 5′ region of the gene of interest and operably linked to the coding sequence of a marker (reporter) gene by recombinant DNA techniques well known to the art. The reporter gene is operably linked downstream of the promoter, so that transcripts initiating at the promoter proceed through the reporter gene. Reporter genes generally encode proteins which are easily measured, including, but not limited to, chloramphenicol acetyl transferase (CAT), beta-glucuronidase (GUS), green fluorescent protein (GFP), beta-galactosidase (beta-GAL), and luciferase.

The construct containing the reporter gene under the control of the promoter is then introduced into an appropriate cell type by transfection techniques well known to the art. To assay for the reporter protein, cell lysates are prepared and appropriate assays, which are well known in the art, for the reporter protein are performed. For example, if CAT were the reporter gene of choice, the lysates from cells transfected with constructs containing CAT under the control of a promoter under study are mixed with isotopically labeled chloramphenicol and acetyl-coenzyme A (acetyl-CoA). The CAT enzyme transfers the acetyl group from acetyl-CoA to the 2- or 3-position of chloramphenicol. The reaction is monitored by thin-layer chromatography, which separates acetylated chloramphenicol from unreacted material. The reaction products are then visualized by autoradiography.

The level of enzyme activity corresponds to the amount of enzyme that was made, which in turn reveals the level of expression from the promoter of interest. This level of expression can be compared to other promoters to determine the relative strength of the promoter under study. In order to be sure that the level of expression is determined by the promoter, rather than by the stability of the mRNA, the level of the reporter mRNA can be measured directly, such as by Northern blot analysis.

Once activity is detected, mutational and/or deletional analyses may be employed to determine the minimal region and/or sequences required to initiate transcription. Thus, sequences can be deleted at the 5′ end of the promoter region and/or at the 3′ end of the promoter region, and nucleotide substitutions introduced. These constructs are then introduced to cells and their activity determined.

In addition to the use of a particular promoter, other types of elements can influence expression of transgenes. In particular, introns have demonstrated the potential for enhancing transgene expression. For example, Callis et al., Genes Dev., 1:1183 (1987) described an intron from the corn alcohol dehydrogenase gene, which is capable of enhancing the expression of transgenes in transgenic plant cells. Similarly, Vasil et al., Mol. Microbiol., 3:371 (1989) described an intron from the corn sucrose synthase gene having similar enhancing activity. The rice actin 1 intron, has been widely used in the enhancement of transgene expression in a number of different transgenic crops. McElroy et al., Mol. Gen. Genet., 231:150 (1991).

Other elements include those that can be regulated by endogenous or exogenous agents, e.g., by zinc finger proteins, including naturally occurring zinc finger proteins or chimeric zinc finger proteins. See, e.g., U.S. Pat. No. 5,789,538, WO 99/48909; WO 99/45132; WO 98/53060; WO 98/53057; WO 98/53058; WO 00/23464; WO 95/19431; and WO 98/54311.

Virtually any DNA composition may be used for delivery to recipient monocotyledonous cells to ultimately produce fertile transgenic plants in accordance with the present invention. For example, DNA segments in the form of vectors and plasmids, or linear DNA fragments, in some instances containing only the DNA element to be expressed in the plant, and the like, may be employed.

In certain embodiments, it is contemplated that one may wish to employ replication-competent viral vectors in monocot transformation. Such vectors include, for example, wheat dwarf virus (WDV) “shuttle” vectors, such as pW1 -11 and PW1-GUS (Ugaki et al., Nucl. Acids Res., 19:371 (1991)). These vectors are capable of autonomous replication in maize cells as well as E. coli , and as such may provide increased sensitivity for detecting DNA delivered to transgenic cells. A replicating vector may also be useful for delivery of genes flanked by DNA sequences from transposable elements such as Ac, Ds, or Mu. It is also contemplated that transposable elements would be useful for introducing DNA fragments lacking elements necessary for selection and maintenance of the plasmid vector in bacteria, e.g., antibiotic resistance genes and origins of DNA replication. It is also proposed that use of a transposable element such as Ac, Ds, or Mu would actively promote integration of the desired DNA and hence increase the frequency of stably transformed cells.

Vectors, plasmids, cosmids, YACs (yeast artificial chromosomes) BACs (bacterial artificial chromosomes) and DNA segments for use in transforming such cells will generally comprise the cDNA, gene or genes which one desires to introduce into the cells. These DNA constructs can further include structures such as promoters, enhancers, polylinkers, or even regulatory genes as desired. The DNA segment or gene chosen for cellular introduction will often encode a protein which will be expressed in the resultant recombinant cells, such as will result in a screenable or selectable trait and/or which will impart an improved phenotype to the regenerated plant. However, this may not always be the case, and the present invention also encompasses transgenic plants incorporating non-expressed transgenes.

DNA useful for introduction into plant cells includes that which has been derived or isolated from any source, that may be subsequently characterized as to structure, size and/or function, chemically altered, and later introduced into plants. An example of DNA “derived” from a source, would be a DNA sequence that is identified as a useful fragment within a given organism, and which is then chemically synthesized in essentially pure form. An example of such DNA “isolated” from a source would be a useful DNA sequence that is excised or removed from said source by chemical means, e.g., by the use of restriction endonucleases, so that it can be further manipulated, e.g., amplified, for use in the invention, by the methodology of genetic engineering. Such DNA is commonly referred to as “recombinant DNA.”

Therefore useful DNA includes completely synthetic DNA, semi-synthetic DNA, DNA isolated from biological sources, and DNA derived from introduced RNA. Generally, the introduced DNA is not originally resident in the plant genotype which is the recipient of the DNA, but it is within the scope of the invention to isolate a gene from a given plant genotype, and to subsequently introduce multiple copies of the gene into the same genotype, e.g., to enhance production of a given gene product such as a storage protein or a protein that confers tolerance or resistance to water deficit.

The introduced DNA includes but is not limited to, DNA from plant genes, and non-plant genes such as those from bacteria, yeasts, animals or viruses. The introduced DNA can include modified genes, portions of genes, or chimeric genes, including genes from the same or different maize genotype. The term “chimeric gene” or “chimeric DNA” is defined as a gene or DNA sequence or segment comprising at least two DNA sequences or segments from species which do not combine DNA under natural conditions, or which DNA sequences or segments are positioned or linked in a manner which does not normally occur in the native genome of untransformed plant.

The introduced DNA used for transformation herein may be circular or linear, double-stranded or single-stranded. Generally, the DNA is in the form of chimeric DNA, such as plasmid DNA, that can also contain coding regions flanked by regulatory sequences which promote the expression of the recombinant DNA present in the resultant plant. For example, the DNA may itself comprise or consist of a promoter that is active in a plant which is derived from a source other than that plant, or may utilize a promoter already present in a plant genotype that is the transformation target.

Generally, the introduced DNA will be relatively small, i.e., less than about 30 kb to minimize any susceptibility to physical, chemical, or enzymatic degradation which is known to increase as the size of the DNA increases. As noted above, the number of proteins, RNA transcripts or mixtures thereof which is introduced into the plant genome is preferably preselected and defined, e.g., from one to about 5-10 such products of the introduced DNA may be formed.

The construction of vectors which may be employed in conjunction with the present invention will be known to those of skill of the art in light of the present disclosure (see e.g., Sambrook et al., Molecular Cloning, A Laboratory Manual (1989); Gelvin et al., Plant Molecular Biology Manual (1990)).

Constructs will also include the gene of interest along with a 3′ end DNA sequence that acts as a signal to terminate transcription and allow for the poly-adenylation of the resultant mRNA. The preferred 3′ elements are contemplated to be those from the nopaline synthase gene of Agrobacterium tumefaciens (Bevan et al., Nucl. Acids Res., 11:369 (1983)), the terminator for the T7 transcript from the octopine synthase gene of Agrobacterium tumefaciens, and the 3′ end of the protease inhibitor I or II genes from potato or tomato. Regulatory elements such as Adh intron 1 (Callis et al., Genes and Develop., 1:1183 (1987)), sucrose synthase intron (Vasil et al., Plant Physiol., 91:1575 (1989)) or TMV omega element (Gallie, et al., The Plant Cell, 1:301(1989)), may further be included where desired.

As the DNA sequence between the transcription initiation site and the start of the coding sequence, i.e., the untranslated leader sequence, can influence gene expression, one may also wish to employ a particular leader sequence. Preferred leader sequences are contemplated to include those which include sequences predicted to direct optimum expression of the attached gene, i.e., to include a preferred consensus leader sequence which may increase or maintain mRNA stability and prevent inappropriate initiation of translation. The choice of such sequences will be known to those of skill in the art in light of the present disclosure. Sequences that are derived from genes that are highly expressed in plants will be most preferred.

Vectors for use in accordance with the present invention may be constructed to include the ocs enhancer element. This element was first identified as a 16 bp palindromic enhancer from the octopine synthase (ocs) gene of Agrobacterium (Ellis et al., EMBO Journal, 6:3203 (1987)), and is present in at least 10 other promoters (Bouchez et al., EMBO Journal, 8:4197 (1989)). The use of an enhancer element, such as the ocs element and particularly multiple copies of the element, will act to increase the level of transcription from adjacent promoters when applied in the context of monocot transformation.

Ultimately, the most desirable DNA segments for introduction into a monocot genome may be homologous genes or gene families which encode a desired trait (e.g., increased yield per acre) and which are introduced under the control of novel promoters or enhancers, etc., or perhaps even homologous or tissue specific (e.g., root-, collar/sheath-, whorl-, stalk-, earshank-, kernel- or leaf-specific) promoters or control elements. Indeed, it is envisioned that a particular use of the present invention will be the targeting of a gene in a constitutive manner or a virus-modulated manner.

Vectors for use in tissue-specific targeting of genes in transgenic plants will typically include tissue-specific promoters and may also include other tissue-specific control elements such as enhancer sequences. Promoters which direct specific or enhanced expression in certain plant tissues will be known to those of skill in the art in light of the present disclosure. These include, for example, the rbcS promoter, specific for green tissue; the ocs, nos and mas promoters which have higher activity in roots or wounded leaf tissue; a truncated (−90 to +8) 35S promoter which directs enhanced expression in roots, an alpha-tubulin gene that directs expression in roots and promoters derived from zein storage protein genes which direct expression in endosperm.

Tissue specific expression may be functionally accomplished by introducing a constitutively expressed gene (all tissues) in combination with an antisense gene that is expressed only in those tissues where the gene product is not desired. For example, a gene coding for the crystal toxin protein from B. thuringiensis (Bt) may be introduced such that it is expressed in all tissues using the 35S promoter from Cauliflower Mosaic Virus. Expression of an antisense transcript of the Bt gene in a maize kernel, using for example a zein promoter, would prevent accumulation of the Bt protein in seed. Hence the protein encoded by the introduced gene would be present in all tissues except the kernel.

Expression of some genes in transgenic plants will be desired only under specified conditions. For example, it is proposed that expression of certain genes that confer resistance to environmental stress factors such as drought will be desired only under actual stress conditions. It is contemplated that expression of such genes throughout a plants development may have detrimental effects. It is known that a large number of genes exist that respond to the environment. For example, expression of some genes such as rbcS, encoding the small subunit-of ribulose bisphosphate carboxylase, is regulated by light as mediated through phytochrome. Other genes are induced by secondary stimuli. For example, synthesis of abscisic acid (ABA) is induced by certain environmental factors, including but not limited to water stress. A number of genes have been shown to be induced by ABA (Skriver and Mundy, Plant Cell, 2:503 (1990)). It is also anticipated that expression of genes conferring resistance to viral infection would be desired only under conditions of actual viral infection. Therefore, for some desired traits inducible expression of genes in transgenic plants will be desired.

Expression of a gene in a transgenic plant may be desired only in a certain time period during the development of the plant. Developmental timing is frequently correlated with tissue specific gene expression. For example, expression of zein storage proteins is initiated in the endosperm about 15 days after pollination.

Additionally, vectors may be constructed and employed in the intracellular targeting of a specific gene product within the cells of a transgenic plant or in directing a protein to the extracellular environment. This will generally be achieved by joining a DNA sequence encoding a transit or signal peptide sequence to the coding sequence of a particular gene. The resultant transit, or signal, peptide will transport the protein to a particular intracellular, or extracellular destination, respectively, and will then be post-translationally removed. Transit or signal peptides act by facilitating the transport of proteins through intracellular membranes, e.g., vacuole, vesicle, plastid and mitochondrial membranes, whereas signal peptides direct proteins through the extracellular membrane.

A particular example of such a use concerns the direction of a herbicide resistance gene, such as the EPSPS gene, to a particular organelle such as the chloroplast rather than to the cytoplasm. This is exemplified by the use of the rbcs transit peptide which confers plastid-specific targeting of proteins. In addition, it is proposed that it may be desirable to target certain genes responsible for male sterility to the mitochondria, or to target certain genes for resistance to phytopathogenic organisms to the extracellular spaces, or to target proteins to the vacuole.

It may be useful to target DNA itself within a cell. For example, it may be useful to target introduced DNA to the nucleus as this may increase the frequency of transformation. Within the nucleus itself it would be useful to target a gene in order to achieve site specific integration. For example, it would be useful to have an gene introduced through transformation replace an existing gene in the cell.

In order to improve the ability to identify transformants, one may desire to employ a selectable or screenable marker gene as, or in addition to, the expressible gene of interest. “Marker genes” are genes that impart a distinct phenotype to cells expressing the marker gene and thus allow such transformed cells to be distinguished from cells that do not have the marker. Such genes may encode either a selectable or screenable marker, depending on whether the marker confers a trait which one can select for by chemical means, i.e., through the use of a selective agent (e.g., a herbicide, antibiotic, or the like), or whether it is simply a trait that one can identify through observation or testing, i.e., by screening (e.g., the R-locus trait). Of course, many examples of suitable marker genes are known to the art and can be employed in the practice of the invention.

Included within the terms selectable or screenable marker genes are also genes which encode a “secretable marker” whose secretion can be detected as a means of identifying or selecting for transformed cells. Examples include markers which encode a secretable antigen that can be identified by antibody interaction, or even secretable enzymes which can be detected by their catalytic activity. Secretable proteins fall into a number of classes, including small, diffusible proteins detectable, e.g., by ELISA; small active enzymes detectable in extracellular solution (e.g., alpha-amylase, beta-lactamase, phosphinothricin acetyltransferase); and proteins that are inserted or trapped in the cell wall (e.g., proteins that include a leader sequence such as that found in the expression unit of extensin or tobacco PR-S).

With regard to selectable secretable markers, the use of a gene that encodes a protein that becomes sequestered in the cell wall, and which protein includes a unique epitope is considered to be particularly advantageous. Such a secreted antigen marker would ideally employ an epitope sequence that would provide low background in plant tissue, a promoter-leader sequence that would impart efficient expression and targeting across the plasma membrane, and would produce protein that is bound in the cell wall and yet accessible to antibodies. A normally secreted wall protein modified to include a unique epitope would satisfy all such requirements.

One example of a protein suitable for modification in this manner is extensin, or hydroxyproline rich glycoprotein (HPRG). For example, the maize HPRG (Steifel et al., The Plant Cell, 2:785 (1990)) molecule is well characterized in terms of molecular biology, expression and protein structure. However, any one of a variety of extensins and/or glycine-rich wall proteins (Keller et al., EMBO Journal, 8:1309 (1989)) could be modified by the addition of an antigenic site to create a screenable marker.

Selectable Markers

Possible selectable markers for use in connection with the present invention include, but are not limited to, a neo gene (Potrykus et al., Mol. Gen. Genet., 199:183 (1985)) which codes for kanamycin resistance and can be selected for using kanamycin, G418, and the like; a bar gene which codes for bialaphos resistance; a gene which encodes an altered EPSP synthase protein (Hinchee et al., Biotech., 6:915 (1988)) thus conferring glyphosate resistance; a nitrilase gene such as bxn from Klebsiella ozaenae which confers resistance to bromoxynil (Stalker et al., Science, 242:419 (1988)); a mutant acetolactate synthase gene (ALS) which confers resistance to imidazolinone, sulfonylurea or other ALS-inhibiting chemicals (European Patent Application 154,204, 1985); a methotrexate-resistant DHFR gene (Thillet et al., J. Biol. Chem., 263:12500 (1988)); a dalapon dehalogenase gene that confers resistance to the herbicide dalapon; or a mutated anthranilate synthase gene that confers resistance to 5-methyl tryptophan. Where a mutant EPSP synthase gene is employed, additional benefit may be realized through the incorporation of a suitable chloroplast transit peptide, CTP (European Patent Application 0,218,571, 1987).

An illustrative embodiment of a selectable marker gene capable of being used in systems to select transformants are the genes that encode the enzyme phosphinothricin acetyltransferase, such as the bar gene from Streptomyces hygroscopicus or the pat gene from Streptomyces viridochromogenes (U.S. patent application Ser. No. 07/565,844, which is incorporated by reference herein). The enzyme phosphinothricin acetyl transferase (PAT) inactivates the active ingredient in the herbicide bialaphos, phosphinothricin (PPT). PPT inhibits glutamine synthetase, (Murakami et al., Mol. Gen. Genet., 205:42 (1986); Twell et al., Plant Physiol., 91:1270 (1989)) causing rapid accumulation of ammonia and cell death. The success in using this selective system in conjunction with monocots was particularly surprising because of the major difficulties which have been reported in transformation of cereals (Potrykus, Trends Biotech., 7:269 (1989)).

Where one desires to employ a bialaphos resistance gene in the practice of the invention, a particularly useful gene for this purpose is the bar or pat genes obtainable from species of Streptomyces (e.g., ATCC No. 21,705). The cloning of the bar gene has been described (Murakami et al., Mol. Gen. Genet., 205:42 (1986); Thompson et al., EMBO Journal, 6:2519 (1987)) as has the use of the bar gene in the context of plants other than monocots (De Block et al., EMBO Journal, 6:2513 (1987); De Block et al., Plant Physiol., 91:694 (1989)).

Screenable Markers

Screenable markers that may be employed include, but are not limited to, a beta-glucuronidase or uidA gene (GUS) which encodes an enzyme for which various chromogenic substrates are known; an R-locus gene, which encodes a product that regulates the production of anthocyanin pigments (red color) in plant tissues (Dellaporta et al., in Chromosome Structure and Function, pp. 263-282 (1988)); a beta-lactamase gene (Sutcliffe, PNAS USA, 75:3737 (1978)), which encodes an enzyme for which various chromogenic substrates are known (e.g., PADAC, a chromogenic cephalosporin); a xylE gene (Zukowsky et al., PNAS USA, 80:1101 (1983)) which encodes a catechol dioxygenase that can convert chromogenic catechols; an α-amylase gene (Ikuta et al., Biotech., 8:241 (1990)); a tyrosinase gene (Katz et al., J. Gen. Microbiol., 129:2703 (1983)) which encodes an enzyme capable of oxidizing tyrosine to DOPA and dopaquinone which in turn condenses to form the easily detectable compound melanin; a β-galactosidase gene, which encodes an enzyme for which there are chromogenic substrates; a luciferase (lux) gene (Ow et al., Science, 234:856 (1986)), which allows for bioluminescence detection; or even an aequorin gene (Prasher et al., Biochem. Biophys. Res. Comm., 126:1259 (1985)), which may be employed in calcium-sensitive bioluminescence detection, or a green fluorescent protein gene (Niedz et al., Plant Cell Reports, 14: 403 (1995)).

Genes from the maize R gene complex are contemplated to be particularly useful as screenable markers. The R gene complex in maize encodes a protein that acts to regulate the production of anthocyanin pigments in most seed and plant tissue. A gene from the R gene complex was applied to maize transformation, because the expression of this gene in transformed cells does not harm the cells. Thus, an R gene introduced into such cells will cause the expression of a red pigment and, if stably incorporated, can be visually scored as a red sector. If a maize line carries dominant allelles for genes encoding the enzymatic intermediates in the anthocyanin biosynthetic pathway (C2, A1, A2, Bz1 and Bz2), but carries a recessive allele at the R locus, transformation of any cell from that line with R will result in red pigment formation. Exemplary lines include Wisconsin 22 which contains the rg-Stadler allele and TR112, a K55 derivative which is r-g, b, P1. Alternatively any genotype of maize can be utilized if the C1 and R alleles are introduced together.

A further screenable marker contemplated for use in the present invention is firefly luciferase, encoded by the lux gene. The presence of the lux gene in transformed cells may be detected using, for example, X-ray film, scintillation counting, fluorescent spectrophotometry, low-light video cameras, photon counting cameras or multiwell luminometry. It is also envisioned that this system may be developed for populational screening for bioluminescence, such as on tissue culture plates, or even for whole plant screening.

Genes of interest are reflective of the commercial markets and interests of those involved in the development of the crop. Crops and markets of interest changes, and as developing nations open up world markets, new crops and technologies will also emerge. In addition, as the understanding of agronomic traits and characteristics such as yield and heterosis increase, the choice of genes for transformation will change accordingly. General categories of genes of interest include, for example, those genes involved in information, such as zinc fingers, those involved in communication, such as kinases, and those involved in housekeeping, such as heat shock proteins. More specific categories of transgenes, for example, include genes encoding important traits for agronomics, insect resistance, disease resistance, herbicide resistance, sterility, grain characteristics, and commercial products. Genes of interest include, generally, those involved in starch, oil, carbohydrate, or nutrient metabolism, as well as those affecting kernel size, sucrose loading, zinc finger proteins, see, e.g., U.S. Pat. No. 5,789,538, WO 99/48909; WO 99/45132; WO 98/53060; WO 98/53057; WO 98/53058; WO 00/23464; WO 95/19431; and WO 98/54311, and the like.

Other sequences which may be linked to the gene of interest which encodes a polypeptide are those which can target to a specific organelle, e.g., to the mitochondria, nucleus, or plastid, within the plant cell. Targeting can be achieved by providing the polypeptide with an appropriate targeting peptide sequence, such as a secretory signal peptide (for secretion or cell wall or membrane targeting, a plastid transit peptide, a chloroplast transit peptide, a mitochondrial target peptide, a vacuole targeting peptide, or a nuclear targeting peptide, and the like. For examples of plastid organelle targeting sequences (see WO 00/12732). Plastids are a class of plant organelles derived from proplastids and include chloroplasts, leucoplasts, aravloplasts, and chromoplasts. The plastids are major sites of biosynthesis in plants. In addition to photosynthesis in the chloroplast, plastids are also sites of lipid biosynthesis, nitrate reduction to ammonium, and starch storage. And while plastids contain their own circular genome, most of the proteins localized to the plastids are encoded by the nuclear genome and are imported into the organelle from the cytoplasm.

Transgenes used with the present invention will often be genes that direct the expression of a particular protein or polypeptide product, but they may also be non-expressible DNA segments. As used herein, an “expressible gene” is any gene that is capable of being transcribed into RNA (e.g., mRNA, antisense RNA, etc.) or translated into a protein, expressed as a trait of interest, or the like, etc., and is not limited to selectable, screenable or non-selectable marker genes. The invention also contemplates that, where both an expressible gene that is not necessarily a marker gene is employed in combination with a marker gene, one may employ the separate genes on either the same or different DNA segments for transformation. In the latter case, the different vectors are delivered concurrently to recipient cells to maximize cotransformation.

The choice of the particular DNA segments to be delivered to the recipient cells will often depend on the purpose of the transformation. One of the major purposes of transformation of crop plants is to add some commercially desirable, agronomically important traits to the plant. Such traits include, but are not limited to, herbicide resistance or tolerance; insect resistance or tolerance; disease resistance or tolerance (viral, bacterial, fungal, nematode); stress tolerance and/or resistance, as exemplified by resistance or tolerance to drought, heat, chilling, freezing, excessive moisture, salt stress; oxidative stress; increased yields; food content and makeup; physical appearance; male sterility; drydown; standability; prolificacy; starch properties; oil quantity and quality; and the like. One may desire to incorporate one or more genes conferring any such desirable trait or traits, such as, for example, a gene or genes encoding herbicide resistance.

In certain embodiments, the present invention contemplates the transformation of a recipient cell with more than one advantageous transgene. Two or more transgenes can be supplied in a single transformation event using either distinct transgene-encoding vectors, or using a single vector incorporating two or more gene coding sequences. For example, plasmids bearing the bar and aroA expression units in either convergent, divergent, or colinear orientation, are considered to be particularly useful. Further preferred combinations are those of an insect resistance gene, such as a Bt gene, along with a protease inhibitor gene such as pinII, or the use of bar in combination with either of the above genes. Of course, any two or more transgenes of any description, such as those conferring herbicide, insect, disease (viral, bacterial, fungal, nematode) or drought resistance, male sterility, drydown, standability, prolificacy, starch properties, oil quantity and quality, or those increasing yield or nutritional quality may be employed as desired.

Herbicide Resistance

The genes encoding phosphinothricin acetyltransferase (bar and pat), glyphosate tolerant EPSP synthase genes, the glyphosate degradative enzyme gene gox encoding glyphosate oxidoreductase, deh (encoding a dehalogenase enzyme that inactivates dalapon), herbicide resistant (e.g., sulfonylurea and imidazolinone) acetolactate synthase, and bxn genes (encoding a nitrilase enzyme that degrades bromoxynil) are good examples of herbicide resistant genes for use in transformation. The bar and pat genes code for an enzyme, phoshinothricin acetyltransferase (PAT), which inactivates the herbicide phosphinothricin and prevents this compound from inhibiting glutamine synthetase enzymes. The enzyme 5-enolpyruvylshikimate 3-phosphate synthase (EPSP Synthase), is normally inhibited by the herbicide N-(phosphonomethyl)glycine (glyphosate). However, genes are known that encode glyphosate-resistant EPSP Synthase enzymes.

These genes are particularly contemplated for use in monocot transformation. The deh gene encodes the enzyme dalapon dehalogenase and confers resistance to the herbicide dalapon. The bxn gene codes for a specific nitrilase enzyme that converts bromoxynil to a non-herbicidal degradation product.

Insect Resistance An important aspect of the present invention concerns the introduction of insect resistance-conferring genes into plants. Potential insect resistance genes which can be introduced include Bacillus thuringiensis crystal toxin genes or Bt genes (Watrud et al., in Engineered Organisms and the Environment (1985)). Bt genes may provide resistance to lepidopteran or coleopteran pests such as European Corn Borer (ECB). Preferred Bt toxin genes for use in such embodiments include the CryIA(b) and CryIA(c) genes. Endotoxin genes from other species of B. thuringiensis which affect insect growth or development may also be employed in this regard.

The poor expression of prokaryotic Bt toxin genes in plants is a well-documented phenomenon, and the use of different promoters, fusion proteins, and leader sequences has not led to significant increases in Bt protein expression (Vaeck et al., Nature, 328:33 (1989); Barton et al., Plant Physiol., 85:1103 (1987)). It is therefore contemplated that the most advantageous Bt genes for use in the transformation protocols disclosed herein will be those in which the coding sequence has been modified to effect increased expression in plants, and more particularly, those in which maize preferred codons have been used. Examples of such modified Bt toxin genes include the variant Bt CryIA(b) gene termed IAb6 (Perlak et al., PNAS-USA, 88:3324 (1991)) and the synthetic CryIA(c) genes termed 1800a and 1800b.

Protease inhibitors may also provide insect resistance (Johnson et al., PNAS-USA, 86:9871 (1989)), and will thus have utility in plant transformation. The use of a protease inhibitor II gene, pinII, from tomato or potato is envisioned to be particularly useful. Even more advantageous is the use of a pinII gene in combination with a Bt toxin gene, the combined effect of which has been discovered by the present inventors to produce synergistic insecticidal activity. Other genes which encode inhibitors of the insects' digestive system, or those that encode enzymes or co-factors that facilitate the production of inhibitors, may also be useful. This group may be exemplified by oryzacystatin and amylase inhibitors, such as those from wheat and barley.

Also, genes encoding lectins may confer additional or alternative insecticide properties. Lectins (originally termed phytohemagglutinins) are multivalent carbohydrate-binding proteins which have the ability to agglutinate red blood cells from a range of species. Lectins have been identified recently as insecticidal agents with activity against weevils, ECB and rootworm (Murdock et al., Phytochemistry, 29:85 (1990); Czapla and Lang, J. Econ. Entomol., 83:2480 (1990)). Lectin genes contemplated to be useful include, for example, barley and wheat germ agglutinin (WGA) and rice lectins (Gatehouse et al., J. Sci. Food Agric., 35:373 (1984)), with WGA being preferred.

Genes controlling the production of large or small polypeptides active against insects when introduced into the insect pests, such as, e.g., lytic peptides, peptide hormones and toxins and venoms, form another aspect of the invention. For example, it is contemplated that the expression of juvenile hormone esterase, directed towards specific insect pests, may also result in insecticidal activity, or perhaps cause cessation of metamorphosis (Hammock et al., Nature, 344:458 (1990)).

Transgenic plants expressing genes which encode enzymes that affect the integrity of the insect cuticle form yet another aspect of the invention. Such genes include those encoding, e.g., chitinase, proteases, lipases and also genes for the production of nikkomycin, a compound that inhibits chitin synthesis, the introduction of any of which is contemplated to produce insect resistant maize plants. Genes that code for activities that affect insect molting, such those affecting the production of ecdysteroid UDP-glucosyl transferase, also fall within the scope of the useful transgenes of the present invention.

Genes that code for enzymes that facilitate the production of compounds that reduce the nutritional quality of the host plant to insect pests are also encompassed by the present invention. It may be possible, for instance, to confer insecticidal activity on a plant by altering its sterol composition. Sterols are obtained by insects from their diet and are used for hormone synthesis and membrane stability. Therefore alterations in plant sterol composition by expression of novel genes, e.g., those that directly promote the production of undesirable sterols or those that convert desirable sterols into undesirable forms, could have a negative effect on insect growth and/or development and hence endow the plant with insecticidal activity. Lipoxygenases are naturally occurring plant enzymes that have been shown to exhibit anti-nutritional effects on insects and to reduce the nutritional quality of their diet. Therefore, further embodiments of the invention concern transgenic plants with enhanced lipoxygenase activity which may be resistant to insect feeding.

The present invention also provides methods and compositions by which to achieve qualitative or quantitative changes in plant secondary metabolites. One example concerns transforming plants to produce DIMBOA which, it is contemplated, will confer resistance to European corn borer, rootworm and several other maize insect pests. Candidate genes that are particularly considered for use in this regard include those genes at the bx locus known to be involved in the synthetic DIMBOA pathway (Dunn et al., Can. J. Plant Sci., 61:583 (1981)).

Further genes encoding proteins characterized as having potential insecticidal activity may also be used as transgenes in accordance herewith. Such genes include, for example, the cowpea trypsin inhibitor (CpTI; Hilder et al., Nature, 330:160 (1987)) which may be used as a rootworm deterrent; genes encoding avermectin (Avermectin and Abamectin., Campbell, W. C., Ed., 1989; Ikeda et al., J. Bacteriol., 169:5612 (1987)) which may prove particularly useful as a corn rootworm deterrent; ribosome inactivating protein genes; and even genes that regulate plant structures.

Environment or Stress Resistance

Improvement of a plant's ability to tolerate various environmental stresses such as, but not limited to, drought, excess moisture, chilling, freezing, high temperature, salt, and oxidative stress, can also be effected through expression of heterologous, or overexpression of homologous genes. Benefits may be realized in terms of increased resistance to freezing temperatures through the introduction of an “antifreeze” protein such as that of the Winter Flounder (Cutler et al., J. Plant Physiol., 135:351 (1989)) or synthetic gene derivatives thereof. Improved chilling tolerance may also be conferred through increased expression of glycerol-3-phosphate acetyltransferase in chloroplasts (Murata et al., 1992; Wolter et al., EMBO Journal, 11:4685 (1992)). Resistance to oxidative stress (often exacerbated by conditions such as chilling temperatures in combination with high light intensities) can be conferred by expression of superoxide dismutase (Gupta et al., PNAS, 90:1629 (1993)), and may be improved by glutathione reductase (Bowler et al., Ann. Rev. Plant Physiol., 43:83 (1992)). Such strategies may allow for tolerance to freezing in newly emerged fields as well as extending later maturity higher yielding varieties to earlier relative maturity zones.

Expression of novel genes that favorably effect plant water content, total water potential, osmotic potential, and turgor can enhance the ability of the plant to tolerate drought. As used herein, the terms “drought resistance” and “drought tolerance” are used to refer to a plants increased resistance or tolerance to stress induced by a reduction in water availability, as compared to normal circumstances, and the ability of the plant to function and survive in lower-water environments, and perform in a relatively superior manner. In this aspect of the invention it is proposed, for example, that the expression of a gene encoding the biosynthesis of osmotically-active solutes can impart protection against drought. Within this class of genes are DNAs encoding mannitol dehydrogenase (Lee and Saier, J. Bacteriol., 153 (1982)) and trehalose-6-phosphate synthase (Kaasen et al., J. Bacteriol., 174:889 (1992)). Through the subsequent action of native phosphatases in the cell or by the introduction and coexpression of a specific phosphatase, these introduced genes will result in the accumulation of either mannitol or trehalose, respectively, both of which have been well documented as protective compounds able to mitigate the effects of stress. Mannitol accumulation in transgenic tobacco has been verified and preliminary results indicate that plants expressing high levels of this metabolite are able to tolerate an applied osmotic stress (Tarczynski et al., cited supra (1992), (1993)).

Similarly, the efficacy of other metabolites in protecting either enzyme function (e.g. alanopine or propionic acid) or membrane integrity (e.g., alanopine) has been documented (Loomis et al., J. Expt. Zool., 252:9 (1989)), and therefore expression of gene encoding the biosynthesis of these compounds can confer drought resistance in a manner similar to or complimentary to mannitol. Other examples of naturally occurring metabolites that are osmotically active and/or provide some direct protective effect during drought and/or desiccation include sugars and sugar derivatives such as fructose, erythritol (Coxson et al., Biotropica, 24:121 (1992)), sorbitol, dulcitol (Karsten et al., Botanica Marina, 35:11 (1992)), glucosylglycerol (Reed et al., J. Gen. Microbiol., 130:1 (1984); Erdmann et al., J. Gen. Microbiol., 138:363 (1992)), sucrose, stachyose (Koster and Leopold, Plant Physiol., 88:829 (1988); Blackman et al., Plant Physiol., 100:225 (1992)), ononitol and pinitol (Vernon and Bohnert, EMBO J., 11:2077 (1992)), and raffinose (Bernal-Lugo and Leopold, Plant Physiol., 98:1207 (1992)). Other osmotically active solutes which are not sugars include, but are not limited to, proline (Rensburg et al., 1993) and glycine-betaine (Wyn-Jones and Storey, In: Physiology and Biochemistry of Drought Resistance in Plants, Paleg et al. (eds.), pp. 171-204 (1981)). Continued canopy growth and increased reproductive fitness during times of stress can be augmented by introduction and expression of genes such as those controlling the osmotically active compounds discussed above and other such compounds, as represented in one exemplary embodiment by the enzyme myoinositol 0-methyltransferase.

It is contemplated that the expression of specific proteins may also increase drought tolerance. Three classes of Late Embryogenic Proteins have been assigned based on structural similarities (see Dure et al., Plant Mol. Biol., 12:475 (1989)). All three classes of these proteins have been demonstrated in maturing (i.e., desiccating) seeds. Within these 3 types of proteins, the Type-II (dehydrin-type) have generally been implicated in drought and/or desiccation tolerance in vegetative plant parts (i.e. Mundy and Chua, EMBO J., 7, 2279 (1988); Piatkowski et al., Plant Physiol., 94:1682 (1990); Yamaguchi-Shinozaki et al., Plant Cell Physiol., 33:217 (1992)). Recently, expression of a Type-III LEA (HVA-1) in tobacco was found to influence plant height, maturity and drought tolerance (Fitzpatrick, Gen. Engineering News, 22:7 (1993)). Expression of structural genes from all three groups may therefore confer drought tolerance. Other types of proteins induced during water stress include thiol proteases, aldolases and transmembrane transporters (Guerrero et al., Plant Mol. Biol., 15:11 (1990)), which may confer various protective and/or repair-type functions during drought stress. The expression of a gene that effects lipid biosynthesis and hence membrane composition can also be useful in conferring drought resistance on the plant.

Many genes that improve drought resistance have complementary modes of action. Thus, combinations of these genes might have additive and/or synergistic effects in improving drought resistance in maize. Many of these genes also improve freezing tolerance (or resistance); the physical stresses incurred during freezing and drought are similar in nature and may be mitigated in similar fashion. Benefit may be conferred via constitutive expression of these genes, but the preferred means of expressing these novel genes may be through the use of a turgor-induced promoter (such as the promoters for the turgor-induced genes described in Guerrero et al. (Plant Molecular Biology, 15:11 (1990)) and Shagan et al., Plant Physiol., 101:1397 (1993), which are incorporated herein by reference). Spatial and temporal expression patterns of these genes may enable maize to better withstand stress.

Expression of genes that are involved with specific morphological traits that allow for increased water extractions from drying soil would be of benefit. For example, introduction and expression of genes that alter root characteristics may enhance water uptake. Expression of genes that enhance reproductive fitness during times of stress would be of significant value. For example, expression of DNAs that improve the synchrony of pollen shed and receptiveness of the female flower parts, i.e., silks, would be of benefit. In addition, expression of genes that minimize kernel abortion during times of stress would increase the amount of grain to be harvested and hence be of value. Regulation of cytokinin levels in monocots, such as maize, by introduction and expression of an isopentenyl transferase gene with appropriate regulatory sequences can improve monocot stress resistance and yield (Gan et al., Science, 270:1986 (1995)).

Given the overall role of water in determining yield, it is contemplated that enabling plants to utilize water more efficiently, through the introduction and expression of novel genes, will improve overall performance even when soil water availability is not limiting. By introducing genes that improve the ability of plants to maximize water usage across a full range of stresses relating to water availability, yield stability or consistency of yield performance may be realized.

Disease Resistance

It is proposed that increased resistance to diseases may be realized through introduction of genes into a plant. It is possible to produce resistance to diseases caused by viruses, bacteria, fungi, root pathogens, insects and nematodes. It is also contemplated that control of mycotoxin producing organisms may be realized through expression of introduced genes.

Resistance to viruses may be produced through gene expression. For example, it has been demonstrated that expression of a viral coat protein in a transgenic plant can impart resistance to infection of the plant by that virus and perhaps other closely related viruses (Cuozzo et al., Bio/Technology, 6:549 (1988), Hemenway et al., EMBO Journal, 7:1273 (1988), Abel et al., Science, 232:738 (1986)). It is contemplated that expression of antisense genes targeted at essential viral functions may impart resistance to the virus. For example, an antisense gene targeted at the gene responsible for replication of viral nucleic acid may inhibit the replication and provide a plant with resistance to the virus. It is believed that interference with other viral functions through the use of antisense genes may also increase resistance to viruses.

It is proposed that increased resistance to diseases caused by bacteria and fungi may be realized through introduction of exogenous genes. It is contemplated that genes encoding so-called “peptide antibiotics,” pathogenesis related (PR) proteins, toxin resistance, and proteins affecting host-pathogen interactions such as morphological characteristics will be useful. Peptide antibiotics are polypeptide sequences which are inhibitory to growth of bacteria and other microorganisms. For example, the classes of peptides referred to as cecropins and magainins inhibit growth of many species of bacteria and fungi. It is proposed that expression of PR proteins in plants may be useful in conferring resistance to bacterial disease. These genes are induced following pathogen attack on a host plant and have been divided into at least five classes of proteins (Bol et al., Ann. Rev. Phytopath., 28:113 (1990)). Included amongst the PR proteins are beta-1,3-glucanases, chitinases, and osmotin and other proteins that are believed to function in plant resistance to disease organisms. Other genes have been identified that have antifungal properties, e.g., UDA (stinging nettle lectin) and hevein (Broakgert et al., Science, 245:1110 (1989); Barkai-Golan et al., Arch. Microbiol., 116:119 (1978)).

Grain Composition or Quality

Genes may be introduced into plants, particularly commercially important cereals such as maize, wheat or rice, to improve the grain for which the cereal is primarily grown. A wide range of novel transgenic plants produced in this manner may be envisioned depending on the particular end use of the grain.

For example, the largest use of maize grain is for feed or food. Introduction of genes that alter the composition of the grain may greatly enhance the feed or food value. The primary components of maize grain are starch, protein, and oil. Each of these primary components of maize grain may be improved by altering its level or composition. Several examples may be mentioned for illustrative purposes but in no way provide an exhaustive list of possibilities.

The protein of many cereal grains is suboptimal for feed and food purposes especially when fed to pigs, poultry, and humans. The protein is deficient in several amino acids that are essential in the diet of these species, requiring the addition of supplements to the grain. Limiting essential amino acids may include lysine, methionine, tryptophan, threonine, valine, arginine, and histidine. Some amino acids become limiting only after the grain is supplemented with other inputs for feed formulations. For example, when the grain is supplemented with soybean meal to meet lysine requirements, methionine becomes limiting. The levels of these essential amino acids in seeds and grain may be elevated by mechanisms which include, but are not limited to, the introduction of genes to increase the biosynthesis of the amino acids, decrease the degradation of the amino acids, increase the storage of the amino acids in proteins, or increase transport of the amino acids to the seeds or grain.

One mechanism for increasing the biosynthesis of the amino acids is to introduce genes that deregulate the amino acid biosynthetic pathways such that the plant can no longer adequately control the levels that are produced. This may be done by deregulating or bypassing steps in the amino acid biosynthetic pathway which are normally regulated by levels of the amino acid end product of the pathway. Examples include the introduction of genes that encode deregulated versions of the enzymes aspartokinase or dihydrodipicolinic acid (DHDP)-synthase for increasing lysine and threonine production, and anthranilate synthase for increasing tryptophan production. Reduction of the catabolism of the amino acids may be accomplished by introduction of DNA sequences that reduce or eliminate the expression of genes encoding enzymes that catalyse steps in the catabolic pathways such as the enzyme lysine-ketoglutarate reductase.

The protein composition of the grain may be altered to improve the balance of amino acids in a variety of ways including elevating expression of native proteins, decreasing expression of those with poor composition, changing the composition of native proteins, or introducing genes encoding entirely new proteins possessing superior composition. DNA may be introduced that decreases the expression of members of the zein family of storage proteins. This DNA may encode ribozymes or antisense sequences directed to impairing expression of zein proteins or expression of regulators of zein expression such as the opaque-2 gene product. The protein composition of the grain may be modified through the phenomenon of cosupression, i.e., inhibition of expression of an endogenous gene through the expression of an identical structural gene or gene fragment introduced through transformation (Goring et al., PNAS, 88:1770 (1991)). Additionally, the introduced DNA may encode enzymes which degrade zeins. The decreases in zein expression that are achieved may be accompanied by increases in proteins with more desirable amino acid composition or increases in other major seed constituents such as starch. Alternatively, a chimeric gene may be introduced that comprises a coding sequence for a native protein of adequate amino acid composition such as for one of the globulin proteins or 10 kD zein of maize and a promoter or other regulatory sequence designed to elevate expression of said protein. The coding sequence of said gene may include additional or replacement codons for essential amino acids. Further, a coding sequence obtained from another species, or, a partially or completely synthetic sequence encoding a completely unique peptide sequence designed to enhance the amino acid composition of the seed may be employed.

The introduction of genes that alter the oil content of the grain may be of value. Increases in oil content may result in increases in metabolizable energy content and density of the seeds for uses in feed and food. The introduced genes may encode enzymes that remove or reduce rate-limitations or regulated steps in fatty acid or lipid biosynthesis. Such genes may include, but are not limited to, those that encode acetyl-CoA carboxylase, ACP-acyltransferase, beta-ketoacyl-ACP synthase, plus other well known fatty acid biosynthetic activities. Other possibilities are genes that encode proteins that do not possess enzymatic activity such as acyl carrier protein. Genes may be introduced that alter the balance of fatty acids present in the oil providing a more healthful or nutritive feedstuff. The introduced DNA may also encode sequences that block expression of enzymes involved in fatty acid biosynthesis, altering the proportions of fatty acids present in the grain such as described below.

Genes may be introduced that enhance the nutritive value of the starch component of the grain, for example by increasing the degree of branching, resulting in improved utilization of the starch in cows by delaying its metabolism.

Besides affecting the major constituents of the grain, genes may be introduced that affect a variety of other nutritive, processing, or other quality aspects of the grain as used for feed or food. For example, pigmentation of the grain may be increased or decreased. Enhancement and stability of yellow pigmentation is desirable in some animal feeds and may be achieved by introduction of genes that result in enhanced production of xanthophylls and carotenes by eliminating rate-limiting steps in their production. Such genes may encode altered forms of the enzymes phytoene synthase, phytoene desaturase, or lycopene synthase. Alternatively, unpigmented white corn is desirable for production of many food products and may be produced by the introduction of DNA which blocks or eliminates steps in pigment production pathways.

Feed or food comprising some cereal grains possesses insufficient quantitities of vitamins and must be supplemented to provide adequate nutritive value. Introduction of genes that enhance vitamin biosynthesis in seeds may be envisioned including, for example, vitamins A, E, B.sub.12, choline, and the like. For example, maize grain also does not possess sufficient mineral content for optimal nutritive value. Genes that affect the accumulation or availability of compounds containing phosphorus, sulfur, calcium, manganese, zinc, and iron among others would be valuable. An example may be the introduction of a gene that reduced phytic acid production or encoded the enzyme phytase which enhances phytic acid breakdown. These genes would increase levels of available phosphate in the diet, reducing the need for supplementation with mineral phosphate.

Numerous other examples of improvement of cereals for feed and food purposes might be described. The improvements may not even necessarily involve the grain, but may, for example, improve the value of the grain for silage. Introduction of DNA to accomplish this might include sequences that alter lignin production such as those that result in the “brown midrib” phenotype associated with superior feed value for cattle.

In addition to direct improvements in feed or food value, genes may also be introduced which improve the processing of grain and improve the value of the products resulting from the processing. The primary method of processing certain grains such as maize is via wetmilling. Maize may be improved though the expression of novel genes that increase the efficiency and reduce the cost of processing such as by decreasing steeping time.

Improving the value of wetmilling products may include altering the quantity or quality of starch, oil, corn gluten meal, or the components of corn gluten feed. Elevation of starch may be achieved through the identification and elimination of rate limiting steps in starch biosynthesis or by decreasing levels of the other components of the grain resulting in proportional increases in starch. An example of the former may be the introduction of genes encoding ADP-glucose pyrophosphorylase enzymes with altered regulatory activity or which are expressed at higher level. Examples of the latter may include selective inhibitors of, for example, protein or oil biosynthesis expressed during later stages of kernel development.

The properties of starch may be beneficially altered by changing the ratio of amylose to amylopectin, the size of the starch molecules, or their branching pattern. Through these changes a broad range of properties may be modified which include, but are not limited to, changes in gelatinization temperature, heat of gelatinization, clarity of films and pastes, and the like. To accomplish these changes in properties, genes that encode granule-bound or soluble starch synthase activity or branching enzyme activity may be introduced alone or combination. DNA such as antisense constructs may also be used to decrease levels of endogenous activity of these enzymes. The introduced genes or constructs may possess regulatory sequences that time their expression to specific intervals in starch biosynthesis and starch granule development. Furthermore, it may be advisable to introduce and express genes that result in the in vivo derivatization, or other modification, of the glucose moieties of the starch molecule. The covalent attachment of any molecule may be envisioned, limited only by the existence of enzymes that catalyze the derivatizations and the accessibility of appropriate substrates in the starch granule. Examples of important derivations may include the addition of functional groups such as amines, carboxyls, or phosphate groups which provide sites for subsequent in vitro derivatizations or affect starch properties through the introduction of ionic charges. Examples of other modifications may include direct changes of the glucose units such as loss of hydroxyl groups or their oxidation to aldehyde or carboxyl groups.

Oil is another product of wetmilling of corn and other grains, the value of which may be improved by introduction and expression of genes. The quantity of oil that can be extracted by wetmilling may be elevated by approaches as described for feed and food above. Oil properties may also be altered to improve its performance in the production and use of cooking oil, shortenings, lubricants or other oil-derived products or improvement of its health attributes when used in the food-related applications. Novel fatty acids may also be synthesized which upon extraction can serve as starting materials for chemical syntheses. The changes in oil properties may be achieved by altering the type, level, or lipid arrangement of the fatty acids present in the oil. This in turn may be accomplished by the addition of genes that encode enzymes that catalyze the synthesis of novel fatty acids and the lipids possessing them or by increasing levels of native fatty acids while possibly reducing levels of precursors. Alternatively DNA sequences may be introduced which slow or block steps in fatty acid biosynthesis resulting in the increase in precursor fatty acid intermediates. Genes that might be added include desaturases, epoxidases, hydratases, dehydratases, and other enzymes that catalyze reactions involving fatty acid intermediates. Representative examples of catalytic steps that might be blocked include the desaturations from stearic to oleic acid and oleic to linolenic acid resulting in the respective accumulations of stearic and oleic acids.

Improvements in the other major cereal wetmilling products, gluten meal and gluten feed, may also be achieved by the introduction of genes to obtain novel plants. Representative possibilities include but are not limited to those described above for improvement of food and feed value.

In addition it may further be considered that the plant be used for the production or manufacturing of useful biological compounds that were either not produced at all, or not produced at the same level, in the plant previously. The novel plants producing these compounds are made possible by the introduction and expression of genes by transformation methods. The possibilities include, but are not limited to, any biological compound which is presently produced by any organism such as proteins, nucleic acids, primary and intermediary metabolites, carbohydrate polymers, etc. The compounds may be produced by the plant, extracted upon harvest and/or processing, and used for any presently recognized useful purpose such as pharmaceuticals, fragrances, industrial enzymes to name a few.

Further possibilities to exemplify the range of grain traits or properties potentially encoded by introduced genes in transgenic plants include grain with less breakage susceptibility for export purposes or larger grit size when processed by dry milling through introduction of genes that enhance gamma-zein synthesis, popcorn with improved popping quality and expansion volume through genes that increase pericarp thickness, corn with whiter grain for food uses though introduction of genes that effectively block expression of enzymes involved in pigment production pathways, and improved quality of alcoholic beverages or sweet corn through introduction of genes which affect flavor such as the shrunken gene (encoding sucrose synthase) for sweet corn.

Non-Protein-Expressing Sequences

DNA may be introduced into plants for the purpose of expressing RNA transcripts that function to affect plant phenotype yet are not translated into protein. Two examples are antisense RNA and RNA with ribozyme activity. Both may serve possible functions in reducing or eliminating expression of native or introduced plant genes.

Genes may be constructed or isolated, which when transcribed, produce antisense RNA that is complementary to all or part(s) of a targeted messenger RNA(s). The antisense RNA reduces production of the polypeptide product of the messenger RNA. The polypeptide product may be any protein encoded by the plant genome. The aforementioned genes will be referred to as antisense genes. An antisense gene may thus be introduced into a plant by transformation methods to produce a novel transgenic plant with reduced expression of a selected protein of interest. For example, the protein may be an enzyme that catalyzes a reaction in the plant. Reduction of the enzyme activity may reduce or eliminate products of the reaction which include any enzymatically synthesized compound in the plant such as fatty acids, amino acids, carbohydrates, nucleic acids and the like. Alternatively, the protein may be a storage protein, such as a zein, or a structural protein, the decreased expression of which may lead to changes in seed amino acid composition or plant morphological changes respectively. The possibilities cited above are provided only by way of example and do not represent the full range of applications.

Genes may also be constructed or isolated, which when transcribed produce RNA enzymes, or ribozymes, which can act as endoribonucleases and catalyze the cleavage of RNA molecules with selected sequences. The cleavage of selected messenger RNA's can result in the reduced production of their encoded polypeptide products. These genes may be used to prepare novel transgenic plants which possess them. The transgenic plants may possess reduced levels of polypeptides including but not limited to the polypeptides cited above that may be affected by antisense RNA.

It is also possible that genes may be introduced to produce novel transgenic plants which have reduced expression of a native gene product by a mechanism of cosuppression. It has been demonstrated in tobacco, tomato, and petunia (Goring et al, PNAS, 88, 1770-1774 (1991); Smith et al. Mol. Gen. Genet., 224:447 (1990); Napoli et al., Plant Cell, 2:279 (1990); van der Krol et al., Plant Cell, 2:291 (1990)) that expression of the sense transcript of a native gene will reduce or eliminate expression of the native gene in a manner similar to that observed for antisense genes. The introduced gene may encode all or part of the targeted native protein but its translation may not be required for reduction of levels of that native protein.

The transgenic plants produced herein are thus expected to be useful for a variety of commercial and research purposes. Transgenic plants can be created for use in traditional agriculture to possess traits beneficial to the grower (e.g., agronomic traits such as resistance to water deficit, pest resistance, herbicide resistance or increased yield), beneficial to the consumer of the grain harvested from the plant (e.g., improved nutritive content in human food or animal feed; increased vitamin, amino acid, and antioxidant content; the production of antibodies (passive immunization) and nutriceuticals), or beneficial to the food processor (e.g., improved processing traits). In such uses, the plants are generally grown for the use of their grain in human or animal foods. All parts of the plants, including stalks, husks, vegetative parts, and the like, may also have utility, including use as part of animal silage or for ornamental purposes. Often, chemical constituents (e.g., oils or starches) of maize and other crops are extracted for foods or industrial use and transgenic plants may be created which have enhanced or modified levels of such components.

Transgenic plants may also find use in the commercial manufacture of proteins or other molecules, where the molecule of interest is extracted or purified from plant parts, seeds, and the like. Cells or tissue from the plants may also be cultured, grown in vitro, or fermented to manufacture such molecules.

The transgenic plants may also be used in commercial breeding programs, or may be crossed or bred to plants of related crop species. Improvements encoded by the expression cassette may be transferred, e.g., from maize cells to cells of other species, e.g., by protoplast fusion.

The transgenic plants may have many uses in research or breeding, including creation of new mutant plants through insertional mutagenesis, in order to identify beneficial mutants that might later be created by traditional mutation and selection. An example would be the introduction of a recombinant DNA sequence encoding a transposable element that may be used for generating genetic variation. The methods of the invention may also be used to create plants having unique “signature sequences” or other marker sequences which can be used to identify proprietary lines or varieties.

Thus, the transgenic plants and seeds according to the invention can be used in plant breeding which aims at the development of plants with improved properties conferred by the expression cassette, such as tolerance of drought, disease, or other stresses. The various breeding steps are characterized by well-defined human intervention such as selecting the lines to be crossed, directing pollination of the parental lines, or selecting appropriate descendant plants. Depending on the desired properties different breeding measures are taken. The relevant techniques are well known in the art and include but are not limited to hybridization, inbreeding, backcross breeding, multiline breeding, variety blend, interspecific hybridization, aneuploid techniques, etc. Hybridization techniques also include the sterilization of plants to yield male or female sterile plants by mechanical, chemical or biochemical means. Cross pollination of a male sterile plant with pollen of a different line assures that the genome of the male sterile but female fertile plant will uniformly obtain properties of both parental lines. Thus, the transgenic seeds and plants according to the invention can be used for the breeding of improved plant lines which for example increase the effectiveness of conventional methods such as herbicide or pesticide treatment or allow to dispense with said methods due to their modified genetic properties. Alternatively new crops with improved stress tolerance can be obtained which, due to their optimized genetic “equipment”, yield harvested product of better quality than products which were not able to tolerate comparable adverse developmental conditions.

The invention will be further described by the following examples which are not intended to limit the scope of the invention.

### EXAMPLES

The invention will be further described by reference to the following detailed examples. These examples are provided for purposes of illustration only, and are not intended to be limiting unless otherwise specified. Standard recombinant DNA and molecular cloning techniques used here are well known in the art and are described in detail in Sambrook et al. (Molecular Cloning: A Laboratory Manual, Cold Spring Harbor Laboratory Press (1989)) and by Ausubel et al. (Current Protocols in Molecular Biology, Greene Publishing (1992)).

### Example 1

### Expression Profiling

1.1 Creation of a GFP Marker Plasmid

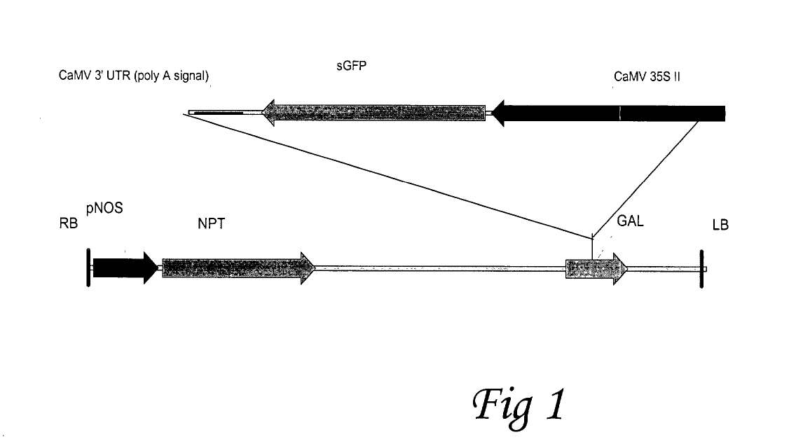

A Green fluorescence protein (GFP) expression cassette is constructed which consists of a sGFP (Reichel et al., P.N.A.S., 93:5888 (1996)), regulated by a duplicated CaMV 35S RNA promoter and CaMV 35S terminator (Goodall G. J., Cell, 58:473 (1989)). The expression cassette is cloned into the GAL polylinker of pBIN19 (Bevan M., N.A.R., 12:8711 (1984)). The plasmid is designated p35S-GFP and shown in FIG. 1.

1.2 Plant Transformation

The GFP vector described in Example 1 is transformed into Arabidopis thaliana plants of ecotype Columbia. The transformation is carried out by taking an “in-Planta Agrobacterium-mediated transformation” approach as described by N. Bechtold (“In planta Agrobacterium-mediated transformation of adult Arabidopsis thaliana plants by vacuum infiltration”; Methods in Molecular Biology, 82: 259-266, (1998)).

1.3 Plant Growth Conditions

Plants are raised axenically on a medium consisting of ½-strength MS salts, 1% (w/v) sucrose, 500 mg/l MES, pH=5.7. The plants are grown from seed in a Phytotron (day length, 10 h; day temperature, 20° C.; night temperature, 16° C.; light Source, Biolux, Osram L58 W/72). Five weeks after germination, leaf tissue is harvested, frozen in liquid nitrogen, and stored at −70 C.

1.4 Evaluation of PTGS in 35S-GFP Transformants

To evaluate PTGS in the resultant 35S-GFP transformants, GFP expression is monitored in transgenic plants by GFP excitation with UV light (approximate range of wavelengths 390 to 480 nm). Selection of transgenic lines showing PTGS is based on absence of GFP expression in mature plants that showed normal GFP expression in earlier stages of plant development. Based on this criterion, two lines designated as 8Z-2 and 5, which are homozygous for the T-DNA insert, show PTGS associated with greatly reduced GFP-mRNA levels detected by RNA blot hybridization as described by Sambrook et al. (Molecular Cloning, 2^{nd} edition. 1989). Line 8Z-2 shows PTGS in approximately 90-96% of sibling plants. Line 5 shows PTGS in approximately 30-50% of sibling plants.

DNA blot hybridization as described by Sambrook et al. (Molecular Cloning. 2^{nd} edition, 1989) reveals that post-transcriptionally silenced line 8Z-2 carries two copies of T-DNA. Further analysis based on polymerase chain reaction (PCR) and utilizing combinations of T-DNA specific primers (Kumar and Fladung (2000) BioTechniques 28: 1128-1137) shows that these two copies are arranged in a direct tandem repeat. Similarly, line 5 is shown to carry one full-length T-DNA and a second, truncated T-DNA copy arranged in an inverted tandem repeat. The genomic position of the T-DNA copies in line 8Z-2 is determined to be chromosome I, BAC F22L4, gene #11 by thermal asymmetric interlaced polymerase chain reaction (TAIL-PCR) (Liu et al. (1995) Plant Journal 8: 457-463) using the T-DNA specific primers LB1 (5′-ttc gga acc acc atc aaa cag g-3′, SEQ ID NO:253), LB2 (5′-ttg ctg caa ctc tct cag ggc c-3′, SEQ ID NO:254), and LB3 (5′-tca gct gtt gcc cgt ctc act-3′, SEQ ID NO:255) and the degenerate primer AD3 (5′-wgt gna gwa nca nag a-3′, where W=A/T and N=G/A/T/C, SEQ ID NO:256). The genomic position of the T-DNA copies in line 5 is determined to be linked to BAC F22L4 on chromosome 1.

1.5 Preparation of RNA

Total RNA is prepared from the frozen samples using Qiagen RNeasy columns (Valencia, Calif.) and precipitated overnight at −20° C. after the addition of 0.25 volumes of 10M LiCl₂. Pellets are washed with 70% EtOH, air dried and resuspended in RNase-free water.

Alternatively, total RNA is prepared using the “Pine Tree method” (Chang et al., 1993) where 1 gram of the ground frozen sample is added to 5 ml of extraction buffer (2% hexadectltrimethylamminium bromide, 2% polyvilylpyrrolidone K 30, 100 mM Tris-HCl (pH 8.0), 25 mM EDTA, 2.0 M NaCl, 0.5 g/L spermidine and 2% beta-mercaptoethanol, previously warmed to 65° C.) and mixed by inversion and vortexing. The solution is extracted two times with an equal volume of chloroform:isoamyl alcohol and precipitated overnight at −20° C. after the addition of 0.25 volumes of 10M LiCl₂. Pellets are washed with 70% EtOH, air dried and resuspended in RNase-free water.

1.6 Preparation of cDNA

First strand cDNA synthesis is accomplished at 42° C. for one hour using 5 μg of total RNA from Arabidopsis tissue, 100 pmol of an oligo dT₍₂₄₎ primer containing a 5′ T7 RNA polymerase promoter sequence [5′-GGCCAGTGAATTGTAATACGACTCACT-ATAGGGAGGCGG-(dT)₂₄-3′] (SEQ ID NO 780) synthesized by Genosys, and SuperScript II reverse transcriptase (RT) (Gibco/BRL).

First strand CDNA synthesis reactions performed with SuperScript II RT are carried out according to the manufacturer's recommendations using 50 mM Tris-HCl (pH 8.3), 75 mM KCl, 3 mM MgCl₂, 10 mM dithiotreitol (DTT), 0.5 mM dNTPs, and 200 units of RT enzyme.

The second cDNA strand is synthesized using 40 units of E. coli DNA polymerase I, 10 units of E. coli DNA ligase, and 2 units of RNase H in a reaction containing 25 mM Tris-HCl (pH 7.5), 100 mM KCl, 5 mM MgCl₂, 10 mM (NH4)SO₄, 0.15 mM β-NAD⁺, 1 mM dNTPs, and 1.2 mM DTT. The reaction proceeded at 16° C. for 2 hours and is terminated using EDTA. Double-stranded cDNA products are purified by phenol/chloroform extraction and ethanol precipitation.

1. 7 Preparation of Biotinvlated cRNA Probes

Synthesized cDNAs (approximately 0.1 μg) are used as templates to produce biotinylated cRNA probes by in vitro transcription using T7 RNA Polymerase (ENZO BioArray High Yield RNA Transcript Labeling Kit). Labeled cRNAs are purified using affinity resin (Qiagen RNeasy Spin Columns) and randomly fragmented to produce molecules of approximately 35 to 200 bases. Fragmentation is achieved by incubation at 94° C. for 35 minutes in a buffer containing 40 mM Tris-acetate, pH 8.1, 100 mM potassium acetate, and 30 mM magnesium acetate.

1.8 Array Hybridization

The labeled samples are mixed with 0.1 mg/mL sonicated herring sperm DNA in a hybridization buffer containing 100 mM 2-N-Morpholino-ethane-sulfonic acid (MES), 1 M NaCl, 20 mM EDTA, 0.01% Tween 20, denatured at 99° C. for 5 min, and equilibrated at 45° C. for 5 min before hybridization. The hybridization mix is then transferred to the Arabidopsis GeneChip genome array (Affymetrix) cartridge and hybridized at 45° C. for 16 h on a rotisserie at 60 rpm.

The hybridized arrays are then rinsed and stained in a fluidics station (Affymetrix). They are first rinsed with wash buffer A (6×SSPE (0.9 M NaCl, 0.06 M NaH₂PO₄, 0.006 M EDTA), 0.01% Tween 20, 0.005% Antifoam) at 25° C. for 10 min and incubated with wash buffer B (100 mM MES, 0.1 M NaCl, 0.01% Tween 20) at 50° C. for 20 min, then stained with Streptavidin Phycoerythrin (SAPE) (100 mM MES, 1 M NaCl, 0.05% Tween 20, 0.005% Antifoam, 10 mg/mL SAPE 2 mg/mL BSA) at 25° C. for 10 min, washed with wash buffer A at 25° C. for 20 min and stained with biotinylated anti-streptavidin antibody at 25° C. for 10 min. After staining, arrays are stained with SAPE at 25° C. for 10 min and washed with wash buffer A at 30° C. for 30 min. The probe arrays are scanned twice and the intensities are averaged with a Hewlett-Packard GeneArray Scanner.

1.9 Data Analysis

GeneChip Suite 3.2 (Affymetrix) is used for data normalization. The overall intensity of all probe sets of each array is scaled to 100 so hybridization intensity of all arrays is equivalent. False positives are defined based on experiments in which samples are split, hybridized to GeneChip expression arrays and the results compared. A false positive is indicated if a probe set is scored qualitatively as an “Increase” or “Decrease” and quantitatively as changing by at least two fold and average difference is greater than 25. A significant change is defined as 2-fold change or above with an expression baseline of 25, which is determined as the threshold level according to the scaling.

The expression data of selected genes are then normalized. Briefly, the median of the expression level within each chip is calculated, and the difference between the average difference and median average difference is used as new value to measure the gene expression level. The expression data are also adjusted across different chip experiments according to the calculated medium. Normalized data (genes and arrays) are analysed by the self organization map (SOM) method (Tamayo et al., P.N.A.S., 96:2907 (1999), and then subject to heirachy cluster analysis (Eisen et al., P.N.A.S., 95:14863 (1998). By the cluster analysis, genes and chip experiments are clustered according to the expression levels.

1.10 Identification of Gene Products that are Modulated by Posttranscriptional Gene Silencing

DNA microarray technology can be used to compare expression patterns of RNA in silent and high-expression tissues of well-characterized 35S-GFP Arabidopsis lines.

Two transgenic lines were generated using the same construct as described in Examples 1 and 2 above. Further selection identified individuals with high expression of transgene GFP, and with low expression of transgene, named H and L (designated as 8Z-2 and 5), respectively. There are two repeats, named 1 and 2. So, for example, AH1 refers to one of the replicates of a transgenic line from event A with high expression of GFP.

By this approach, those genes could be identified which show reproducible expression difference between the H and L lines, regardless of their genetic background (A and B).

RNA expression patterns were determined by comparision of replicate RNA samples from different pairs of high-expressing and silent leaves in comparable physiological and developmental states. RNA expression patterns were also compared from samples obtained from tissues harvested at different times in the silencing process to detect genes expressed at specific stages during initiation, maintenance and systemic spread of PTGS.

1.11 Analysis of RNA Profiling Results

1.11.1 Expression Profiles of the Two Transgenic Lines

A comparitive analysis was conducted among eight samples using AH1 as a baseline. Genes displaying significant changes were collected. These genes showed at least a 2-fold change, average difference above 25 (determined noise level), and were present in at least 1 sample. The expression data of selected genes were then normalized as described above.

By this approach, a total of 1830 genes from four samples were clustered into two groups according to the genetic background of the cell lines rather than the degree of PTGS. Two cell lines showed very distinct gene expression patterns. However, no obvious difference in gene expression pattern was observed between highly expressed lines and silent lines. A similar pattern was discovered using more stringent criteria: at least a 5-fold change, average difference above 50, and present at least in one sample. The clustering was again based on the genetic background of the transgenic lines. 44 genes were analyzed according to this method. The results indicated that a different approach was needed to identify genes associated with PTGS.

1.11.2 Difference in Gene Expression Between Silent and Highly Expressed Lines

In order to detect the differences in gene expression in silent and highly expressed lines, a different strategy was used. Pair-wise comparisons were conducted between each silent and highly expressed cell line pair using the above stated selection criteria. Selected genes were pooled together, and SOM and heirachy cluster analyses were performed to identify the common expression pattern in different cell lines and replicates.

Using low stringent criteria, a total of 823 genes were selected. Four very distinct clusters of genes were identified. Two of the clusters contain genes preferentially expressed in silent lines, while the other two clusters contain genes preferentially expressed in the highly expressed lines. The range and average of the expression levels within each of the clusters were calculated and plotted in the inserted graph. An additional cluster analysis was performed with the selected data containing genes from these four clusters. 28 genes preferentially expressed in silent lines and 21 genes preferentially expressed in highly expressed lines were selected.

Using the high stringent criteria, a total of 145 genes were selected and analyzed.

1.11.3 Statistical Analysis

The relative expression of genes in high-expressing (H) and silent (S) Arabidopsis plants transformed with a 35S-green fluorescent protein (GFP) reporter gene and in wild-type (W) Arabidopsis was compared. Normalized expression values were used to calculate average expression values for each probe set. Probe sets were selected that showed significant differences (one-sided t-test of means, p<0.05 [*] and p<0.005 [**], 3 replicates) between S and W samples or S and H samples. The relative expression levels S/W and S/H were calculated for the data subsets.

Two classes of PTGS-related genes were identified: 1) Genes induced in association with PTGS showing ca. 2-fold higher expression in S relative to H or relative to W (upregulated in silent lines). 2) Genes down-regulated in association with PTGS showing ca. 2-fold lower expression in S relative to H or relative to W (downregulated in silent lines).

43 candidate genes were identified that showed increased expression in silent plants and 51 candidate genes showing decreased expression in silent plants.

1.12 Gene Classes

Based on blast searches of DNA databases, these genes encode proteins or protein domains which fall into several classes: 1) Silencing-related RNA and DNA metabolism (e.g., RNA helicases, RNAses, reverse transcriptase, histones, histone acetyltransferases), 2) signal transduction (protein kinases, receptors, and calmodulin), 3) transcription factors, 4) stress-related and pathogenesis-related proteins, 5) general metabolism, and 6) Proteins without known function. Strongly induced genes that encode a reverse transcriptase-like protein (CAB43904) implicate synthesis of novel DNAs in PTGS. Induced genes that encode a histone acetyltransferase-like protein (CAA18725) provide a link between PTGS and transcriptional gene silencing. The strong correlation between silencing and changes in expression of a variety of stress-related and pathogenesis-related proteins indicates that PTGS involves plant responses similar to those associated with defense against microbial pathogens.

### Example 2

### Identification of Ortholozous Genes in Rice, Wheat Banana and Maize

In addition, the identification of the Arabidopsis genes provides a means to identify the corresponding homologs and orthologs in other plants, including commercially valuable food crops such as wheat, rice, banana, soy, and barley, maize and ornamental plants. BLASTN and BLASTP searches can be performed to identify such sequences. Searches to identify such sequences revealed the polynucleotide sequences set forth in Table 6

The Arabidopsis sequences were compared to two sets or rice sequences to identify homologs. One contained rice gene prediction mRNA sequences and the other rice cDNA ORF sequences. The comparison algorithm was a translated BLAST search, tblastx. The BLAST results were post processed using SCAN software with the default parameters. This processed data was then parsed to retrieve the top rice hit based on E-value. These rice sequences were then compared to sets of wheat, maize and banana clustered cDNA's using the same software process to retrieve the top hits from each set.

TABLE 1
Genes up-regulated in silent lines

| At | Gene | | Rice |
|---|---|---|---|
| SEQ | Chip | | SEQ |
| ID NO: | Probe No: | Description and public homologs | ID NO |
| 5 | 20297_AT | : gb\|AAD30627.1\|AC007153_19 (AC007153) Similar to | 201 |
| | | indole-3-acetate beta-glucosyltransferase [Arabidopsis | |
| | | thaliana] | |
| | | -AAF61647.1 AF190634 Nicotiana tabacum UDP- | |
| | | glucose:salicylic acid glucosyltransferase. | |
| | | -AAL09350.1 AF304430 Brassica napus | |
| | | thiohydroximate S-glucosyltransferase. | |
| 9 | 19983_AT | : Similar to Rab protein [Human] | 157 |
| 9 | 19983_AT | : Similar to Rab protein [Human] | 133 |
| 11 | 12951_AT | : gb\|AAD32867.1\|AC005489_5 (AC005489) F14N23.5 | |
| | | [Arabidopsis thaliana] | |
| 17 | 14176_AT | : No hits found less than or equal to 1e-15. | |
| 21 | 14170_AT | : gb\|AAF29406.1\|AC022354_5 (AC022354) unknown | 309 |
| | | protein [Arabidopsis thaliana] | |
| 21 | | | 29 |
| 23 | 18115_AT | : gb\|AAC64891.1\| (AC005388) Similar to T11J7.13 | 311 |
| | | gi\|2880051 putative protein kinase from Arabidopsis | |
| | | thaliana BAC gb\|AC002340 | |
| 23 | | | 39 |
| 35 | 14636_S_{—} | : gb\|AAF21072.1\|AC013258_10 (AC013258) thaumatin- | 315 |
| | AT, | like protein [Arabidopsis thaliana] | |
| | 16153_S_{—} | -AAF06347.1 AF195654 Vitis vinifera SCUTL2. | |
| | AT | thaumatin-like protein. | |
| | | -BAA28872.1 AB006009 Pyrus pyrifolia thaumatin- | |
| | | like protein precursor. PsTL1. | |
| 35 | | | 109 |
| 39 | 17073_S_{—} | : gb\|AAD20078.1\| (AC006836) putative steroid | 113 |
| | AT14643_{—} | sulfotransferase [Arabidopsis thaliana] | |
| | S_AT | -AAC63113.1 AF000307 Brassica napus steroid | |
| | | sulfotransferase 3. BnST3. | |
| | | -AAC63112.1 AF000306 Brassica napus steroid | |
| | | sulfotransferase 2. BnST2. | |
| 39 | | | 331 |
| 41 | 15950_AT | : gb\|AAD26911.1\|AC006429_1 (AC006429) unknown | 159 |
| | | protein [Arabidopsis thaliana] | |
| | | -AAD03487.1 AF028841 Medicago sativa proline- | |
| | | rich cell wall protein. | |
| | | -CAA47812.1 X67427 Pisum sativum ptxA. | |
| 41 | | | 49 |
| 45 | 15846_AT | : gb\| AAD15461.1\| (AC006067) unknown protein | |
| | , | [Arabidopsis thaliana] | |
| | 14704_S_{—} | | |
| | AT15847_{—} | | |
| | G_AT | | |
| 59 | 17894_AT | : gb\|AAD08938.1\| (AC005724) unknown protein | |
| | | [Arabidopsis thaliana] | |
| 61 | 13706_S_{—} | : gb\|AAD08939.1\| (AC005724) putative trehalose-6- | 329 |
| | AT | phosphate synthase [Arabidopsis thaliana] | |
| 61 | | | 81 |
| 65 | 20079_AT | : gb\|AAD21753.1\| (AC006569) putative AP2 domain | 327 |
| | | transcription factor [Arabidopsis thaliana] | |
| | | -BAA97123.1 AB016265 Nicotiana sylvestris ERF | |
| | | (EREBP); ethylene-responsive element binding factor for | |
| | | basic PR (Pathogenesis-related) gene of higher plant, | |
| | | ethylene-responsive element binding factor. nserf3. | |
| 65 | | | 139 |
| 69 | 20676_G_{—} | : gb\|AAD20920.1\| (AC006234) beta-expansin | 275 |
| | AT | [Arabidopsis thaliana] | |
| | | -AAA50175.1 U03860 Glycine max cytokinin | |
| | | induced message. cim1. submitter comments: has | |
| | | homology to a perennial ryegrass pollen allergen | |
| | | (M57476); extracellular protein;cytokinin induced mRNA. | |
| | | -AAG52887.1 AF333386 Nicotiana tabacum beta- | |
| | | expansin-like protein. PPAL. pollen allergen-like protein. | |
| 69 | | | 43 |
| 73 | 17386_AT | : gb\|AAD29802.1\|AC006264_10 (AC006264) putative | |
| | | proline-rich protein [Arabidopsis thaliana] | |
| | | -CAA04449.1 AJ000997 Solanum tuberosum | |
| | | proline-rich protein. gpp1 | |
| 75 | 15863_AT | : gb\|AAD20401.1\| (AC007019) unknown protein | 295 |
| | | [Arabidopsis thaliana] | |
| 75 | | | 121 |
| 81 | 14423_S_{—} | : gb\|AAD23672.1\|AC007070_21 (AC007070) unknown | 163 |
| | AT | protein [Arabidopsis thaliana] | |
| 81 | | | 129 |
| 83 | 15576_S_{—} | : gb\|AAC42256.1\| (AC005395) putative CCCH-type zinc | 65 |
| | AT | finger protein [Arabidopsis thaliana] | |
| 83 | | | 263 |
| 85 | 19951_AT | : gb\|AAC42241.1\| (AC005395) unknown protein | |
| | | [Arabidopsis thaliana] | |
| 91 | 12497_AT | : putative receptor-like protein kinase [Arabidopsis | 337 |
| | | thaliana] | |
| | | -CAC20842.1 AJ250467 Pinus sylvestris receptor | |
| | | protein kinase. upk. | |
| 91 | | | 131 |
| 107 | 19135_AT | : gb\|AAF14581.1\|AF188363_1 (AF188363) AnnAt4 | 289 |
| | | [Arabidopsis thaliana] | |
| | | -CAA06492.1 AJ005347 Cicer arietinum calcium- | |
| | | binding protein. annexin. | |
| 107 | | | 55 |
| 111 | 19848_S_{—} | : dbj\|BAA08282.1\| (D45848) calmodulin-related protein | 251 |
| | AT | [Arabidopsis thaliana] | |
| | | -AAF73157.1 AF150059 Brassica napus | |
| | | calmodulin. CaM1. involved in seed germination. | |
| | | -CAA61980.1 X89890 Bidens pilosa Calmodulin. | |
| 111 | | | 3 |
| 125 | 19315_AT | : gb\|AAC62875.1\| (AC005397) putative AP2 domain | 175 |
| | | transcription factor [Arabidopsis thaliana] | |
| 125 | | | 21 |
| 129 | 13019_S_{—} | : gb\|AAC34233.1\| (AC00441 1) putative RAV-like B3 | 317 |
| | AT | domain DNA binding protein [Arabidopsis thaliana] | |
| 129 | | | 57 |
| 131 | 14062_AT | : gb\|AAC63633.1\| (AC005309) unknown protein | 219 |
| | | [Arabidopsis thaliana] | |
| | | -AAD51854.1 AF178990 Vitis riparia stress | |
| | | related protein. SRP. | |
| | | -CAA11305.1 AJ223390 Hevea brasiliensis Hev b 3. hev | |
| | | b 3. | |
| 131 | | | 75 |
| 141 | 12541_AT | : emb\|CAA61485.1\| (X89192) DNA binding protein | 215 |
| | | [Arabidopsis thaliana] | |
| | | -CAB89831.1 AJ242853 Solanum tuberosum DNA | |
| | | binding protein. Dof zinc finger protein, dof1. | |
| | | -BAA78575.1 AB028132 Oryza sativa Dof zinc | |
| | | finger protein. | |
| 141 | | | 115 |
| 145 | 13212_S_{—} | : “emb\|CAB68132.1\| (AL137080) beta-1, 3-glucanase 2 | 221 |
| | AT, | (BG2) [Arabidopsis thaliana]” | |
| | 16578_S_{—} | -AAL30426.1 AF435089 Prunus persica beta-1,3- | |
| | AT | glucanase. Gns3. | |
| | | -AAL30425.1 AF435088 Prunus persica beta-1,3- | |
| | | glucanase. Gns2. | |
| 145 | | | 105 |
| 147 | 20345_AT | : “gb\|AAC72865.1\| (AF104919) similar to class I | 237 |
| | | chitinases (Pfam: PF00182, E = 1.2e-142, N = 1) | |
| | | [Arabidopsis thaliana]” | |
| | | -CAA57773.1 X82329 Arachis hypogaea chitinase | |
| | | (class II). chi2;1. | |
| | | -AAD54935.1 AF141373 Petroselinum crispum | |
| | | random hydrolysis of1,4-beta-acetamido-2-deoxy-D- | |
| | | glucoside linkages in chitin. chitinase precursor. Chi1-1. | |
| | | class II. | |
| 151 | 19181_S_{—} | : gb\|AAC39464.1\| (AF053065) late embryogenesis | 261 |
| | AT | abundant protein homolog [Arabidopsis thaliana] | |
| 151 | | | 47 |
| 153 | 17834_AT | : gb\|AAC28197.1\| (AF075598) contains similarity to | 243 |
| | | reverse transcriptases [Arabidopsis thaliana] | |
| | | -CAA73821.1 Y13391 Beta lomatogona reverse | |
| | | transcriptase. | |
| 167 | 18983_S_{—} | : emb\|CAB41722.1\| (AL049730) pEARLI 1-like protein | |
| | AT | [Arabidopsis thaliana] | |
| | | -BAA11855.1 D83227 Populus nigra extensin like | |
| | | protein. | |
| | | -BAA11854.1 D83226 Populus nigra extensin like | |
| | | protein. | |
| 169 | 20604_AT | : emb\|CAB40776.1\| (AL049608) putative protein | 291 |
| | | [Arabidopsis thaliana] | |
| | | -AAL25128.1 AF432499 Oryza sativa cellulose | |
| | | synthase-like protein OsCs1A9. | |
| | | -AAL25127.1 AF432498 Oryza sativa cellulose | |
| | | synthase-like protein OsCs1A6. | |
| 171 | 12540_S_{—} | : emb\|CAB10218.1\| (Z97336) hypothetical protein | 283 |
| | AT | [Arabidopsis thaliana] | |
| 179 | 14552_S_{—} | : emb\|CAA18725.1\| (AL022603) Lsd1 like protein | 151 |
| | AT | [Arabidopsis thaliana] | |
| 179 | | | 141 |
| 181 | 14114_F_{—} | : emb\|CAB36808.1\| (AL035527) subtilisin-like protease | 271 |
| | AT | [Arabidopsis thaliana] | |
| | | -AAG09442.1 AF200467 Oryza sativa subtilase. | |
| | | serine protease; subtilisin-like serine protease;SP1. | |
| | | -CAA10987.1 AJ222782 Hordeum vulgare | |
| | | subtilisin-like protease. | |
| 183 | 15319_AT | : emb\|CAA20471.1\| (AL031326) putative protein | 267 |
| | | [Arabidopsis thaliana] | |
| | | -BAA90634.1 AP001129 Oryza sativa ESTs | |
| | | AU029388(E30287),D49277(S16474) correspond to a | |
| | | region of the predicted gene.; Similar to | |
| | | Arabidopsis thaliana DNA chromosome 4, BAC clone | |
| | | F16G20, picA protein.(AL031326). | |
| 183 | | | 79 |
| 191 | 12302_AT | : emb\|CAB43904.1\| (AL078469) putative protein | 301 |
| | | [Arabidopsis thaliana] | |
| | | -AAL31076.1 AC091749 Oryza sativa putaive copia- | |
| | | like retrotransposon polyprotein,5′-partial. | |
| | | OSJNBb0008A05.28. | |
| 205 | 18355_AT | : emb\|C AA21470.1\| (AL031986) putative protein | 301 |
| | | [Arabidopsis thaliana] | |
| | | -AAL33552.1 AF436851 Cucumis melo RING-H2 | |
| | | zinc finger protein. | |
| 205 | | | 301 |
| 207 | 14032_AT | : emb\|CAB38204.1\| (AL035601) cytochrome P450-like | 145 |
| | | protein [Arabidopsis thaliana] | |
| | | -BAA22422.1 AB001379 Glycyrrhiza echinata | |
| | | cytochrome P450. CYP81E1. | |
| | | -BAA74465.1 AB022732 Glycyrrhiza echinata | |
| | | cytochrome P450. CYP Ge-31. | |
| 215 | 14646_S_{—} | : emb\|CAB43637.1\| (AL050351) AtRer1A [Arabidopsis | 325 |
| | AT | thaliana] | |
| 215 | | | 125 |
| 217 | : | : emb\|CAA18763.1\| (AL022605) puative protein | 245 |
| | 15243_AT | [Arabidopsis thaliana] | |
| 219 | 20480_S_{—} | : emb\|CAB38908.1\| (AL035708) cytochrome P450-like | 281 |
| | AT, | protein [Arabidopsis thaliana] | |
| | 20479_I_{—} | -AAD03415.1 AF069494 Sinapis alba converts | |
| | AT | tyrosine topara-hydrophenylacetaldoxime inpara- | |
| | | hydroxybenzylglucosinolate biosynthesis. cytochrome | |
| | | P450. CYP79B1. | |
| | | -AAA85440.1 U32624 Sorghum bicolor cytochrome | |
| | | P-450. CYP79. P450TYR;N-hydroxylase. | |
| 219 | | | 101 |
| 223 | 13710_AT | : No hits found less than or equal to 1e-15. | |
| 225 | 13277_I_{—} | : emb\|CAA74399.1\| (Y14070) Heat Shock Protein 17.6A | 273 |
| | AT | [Arabidopsis thaliana] | |
| | | -AAC14577.1 U72396 Lycopersicon esculentum | |
| | | class II small heat shock protein Le-HSP17.6. heat | |
| | | treatment. | |
| | | -AAA33670.1 M33901 Pisum sativum 17.7 kDa | |
| | | heat shock protein (hsp17.7). | |
| 225 | | | 89 |
| 229 | 15141_S_{—} | : dbj\|BAA22096.1\| (D85191) vegetative storage protein | 171 |
| | AT | [Arabidopsis thaliana] | |
| | | -CAA11075.1 AJ223074 Glycine max acid | |
| | | phosphatase. | |
| | | -AAA33967.1 M76981 Glycine max vegetative | |
| | | storage protein. vspA. | |
| 233 | 14116_AT | : “gb\|AAC26243.1\| (AF077407) contains similarity to | 247 |
| | | sugar transporters (Pfam: sugar_tr.hmm, score: 395.39) | |
| | | [Arabidopsis thaliana]” | |
| | | -CAB52689.1 AJ132224 Lycopersicon esculentum | |
| | | hexose transporter. ht2. | |
| | | -CAA09419.1 AJ010942 Lycopersicon esculentum | |
| | | hexose transporter protein. | |
| 233 | | | 51 |
| 235 | 18206_AT | : dbj\|BAA85261.1\| (AB033294) dihydroflavonol 4- | 149 |
| | | reductase [Arabidopsis thaliana] | |
| | | -CAA72420.1 Y11749 Vitis vinifera | |
| | | dihydroflavonol 4-reductase. dfr1. | |
| | | -CAA53578.1 X75964 Vitis vinifera | |
| | | dihydroflavonol reductase. DFR | |
| 235 | | | 93 |
| 237 | 20034_I_{—} | : emb\|CAB42601.1\| (A71607) unnamed protein product | 297 |
| | AT | [Arabidopsis thaliana] | |
| | | -BAB33033.1 AB056448 Vigna unguiculata CPRD2. | |
| | | -BAB68539.1 AB071704 Daucus carota (S)- | |
| | | reticuline oxidase-like protein | |
| 243 | 16620_S_{—} | : gb\|AAC05572.1\| (AF051338) xyloglucan | 149 |
| | AT | endotransglycosylase related protein [Arabidopsis | |
| | | thaliana] | |
| | | -AAA81350.1 L22162 Glycine max brassinosteroid- | |
| | | regulated protein. This translated sequence has significant | |
| | | homologyto MER5 _ARATH (pir JQ1022) and | |
| | | TMNXG1A_1 (gp X68254). | |
| 243 | | | 93 |
| 251 | 20471_AT | : AP2 domain containing protein RAP2.1 [Arabidopsis | 269 |
| | | thaliana] | |
| | | | 11 |
| | 12345_at | gb\|AAB67985.1\| (L36246) anoxia-induced protein | |
| | (L36246.2 | [Arabidopsis thaliana] | |
| | _AT) | -BAA78738.1 AB023482 Oryza sativa EST | |
| | | AU055776(S20048) corresponds to a region of | |
| | | thepredicted gene.; Similar to Arabidopsis thaliana | |
| | | AP2domain containing protein RAP2.10 mRNA, | |
| | | partialcds.(AF003103). | |
| | | -AAF76898.1 AF274033 Atriplex hortensis apetala2 | |
| | | domain-containing protein. | |

TABLE 2
Genes down-regulated in silent lines

| AT | Gene | | Rice |
|---|---|---|---|
| SEQ | Chip | | SEQ |
| ID NO | Probe No: | Description | ID NO |
| 1 | 16053_I_{—} | : emb\|CAA74639.1\| (Y14251) glutathione S-transferase | 189 |
| | AT, | [Arabidopsis thaliana] | |
| | 16054_S_{—} | -CAA55039.1 X78203 Hyoscyamus muticus | |
| | AT | glutathione transferase. | |
| 1 | | | 111 |
| 3 | 13604_AT | : gb\|AAB70431.1\| (AC000104) F19P19.10 [Arabidopsis | 339 |
| | | thaliana] | |
| 7 | 19078_AT | : gb\|AAC14036.1\| (AC003981) F22O13.10 [Arabidopsis | 249 |
| | | thaliana] | |
| 13 | 14043_AT | : gb\|AAD39282.1\|AC007576_5 (AC007576) Similar to | |
| | | DNA-binding proteins [Arabidopsis thaliana] | |
| | | -AAD16139.1 AF096299 Nicotiana tabacum DNA- | |
| | | binding protein 2. WRKY2. transcription factor; contains | |
| | | WRKY DNA-binding domain. | |
| | | -AAC37515.1 L44134 Cucumis sativus SPF1-like | |
| | | DNA-binding protein. | |
| 15 | 19424_AT | : gb\|AAC00588.1\| (AC002396) glucose-6-phosphate 1- | 209 |
| | | dehydrogenase [Arabidopsis thaliana] | |
| | | -AAB69317.1 AF012861 Petroselinum crispum | |
| | | plastidic glucose-6-phosphate dehydrogenase. pG6PDH. | |
| | | -AAF87216.1 AF231351 Nicotiana tabacum plastidic | |
| | | glucose 6-phosphate dehydrogenase. G6PDHP2. | |
| 19 | 18217_G_{—} | : gb\|AAF24959.1\|AC012375_22 (AC012375) T22C5.18 | 187 |
| | AT | [Arabidopsis thaliana] | |
| | | -AAD26942.1 AF119050 Datisca glomerata zinc- | |
| | | finger protein 1. zfp1. DgZFP1. | |
| | | -BAA05079.1 D26086 Petunia x hybrida zinc-finger | |
| | | protein. | |
| 19 | | | 123 |
| 25 | 16537_S_{—} | : dbj\|BAA28953.1\| (AB008111) Atrboh F [Arabidopsis | 259 |
| | AT | thaliana] | |
| | | -BAB68079.1 AP003560 Oryza sativa cytochrome | |
| | | b245 beta chain homolog rbohA. B1060H01.12. contains | |
| | | ESTsD40466(S2474), C72826(E2330), AU085905(E2330). | |
| | | -BAB70750.1 AB050660 Solanum tuberosum | |
| | | respiratory burst oxidase homolog. StrbohA. | |
| 25 | | | 13 |
| 27 | 14964_AT | : gb\|AAB60905.1\| (AC001229) F5I14.4 gene product | |
| | | [Arabidopsis thaliana] | |
| 29 | 15116_F_{—} | : gb\|AAD28243.1\|AF121356_1 (AF121356) | 205 |
| | AT | peroxiredoxin TPx2 [Arabidopsis thaliana] | |
| | | -AAD33602.1 AF133302 Brassica rapa subsp. | |
| | | pekinensis type 2 peroxiredoxin. PrxII. new type. | |
| | | -AAL35363.1 AF442385 Capsicum annuum | |
| | | thioredoxin peroxidase. CAPOT1 | |
| 29 | | | 7 |
| 31 | 14656_I_{—} | : gb\|AAC41678.1\| (L41244) thionin [Arabidopsis | |
| | AT | thaliana] | |
| | | -AAF21800.1 AF090836 Brassica rapa subsp. | |
| | | pekinensis | |
| | | : thionin. | |
| 33 | 17077_S_{—} | : gb\|AAA67927.1\| (U13949) AtHSP101 [Arabidopsis | 253 |
| | AT, | thaliana] | |
| | 13274_S_{—} | -AAA66338.1 L35272 Glycine max heat shock | |
| | AT | protein. SB100. 100 kDa. | |
| | | -AAC83688.2 AF083343 Nicotiana tabacum 101 kDa | |
| | | heat shock protein. HSP101 | |
| 33 | | | 119 |
| 37 | 14679_S_{—} | : gb\|AAD17441.1\| (AC006284) putative WRKY DNA- | 195 |
| | AT | binding protein [Arabidopsis thaliana] | |
| | | -AAD16139.1 AF096299 Nicotiana tabacum DNA- | |
| | | binding protein 2. WRKY2. transcription factor; contains | |
| | | WRKY DNA-binding domain. | |
| | | -AAC37515.1 L44134 Cucumis sativus SPF1-like | |
| | | DNA-binding protein. | |
| 37 | | | 63 |
| 43 | 14377_I_{—} | : gb\|AAD20103.1\| (AC006304) hypothetical protein | |
| | AT | [Arabidopsis thaliana] | |
| 47 | 17128_S_{—} | : gb\|AAC69381.1\| (AC005398) pathogenesis-related PR- | 173 |
| | AT, | 1-like protein [Arabidopsis | |
| | 14635_S_{—} | -AAB06458.1 U64806 Brassica napus | |
| | AT | pathogenesis-related protein PR1. Ypr1. | |
| | | -AAB01666.1 U21849 Brassica napus PR-1a. | |
| | | LSC94. | |
| 49 | 12768_AT | : gb\|AAD41977.1\|AC006438_9 (AC006438) unknown | |
| | | protein [Arabidopsis thaliana] | |
| 51 | 17832_S_{—} | : gb\|AAB82769.1\| (U94998) class 1 non-symbiotic | 165 |
| | AT | hemoglobin [Arabidopsis thaliana] | |
| | | -AAL09463.1 AF329368 Gossypium hirsutum non- | |
| | | symbiotic hemoglobin class 1. GLB1. | |
| | | -AAG29748.1 AF172172 Medicago sativa non- | |
| | | symbiotic hemoglobin. MHB1. | |
| 51 | | | 87 |
| 53 | 14965_AT | : gb\|AAB86507.1\| (AC002329) unknown protein | |
| | | [Arabidopsis thaliana] | |
| 55 | 19554_AT | : gb\|AAD32908.1\|AC007584_6 (AC007584) putative | 169 |
| | | mitotic control protein dis3 [Arabidopsis thaliana] | |
| | | -BAA85401.1 AP000615 Oryza sativa EST | |
| | | AU068209(C12438) corresponds to a region of | |
| | | the predicted gene.; similar to Dis3p protein - | |
| | | human.(JE0110). | |
| 57 | 15695_S_{—} | : gb\|AAD20121.1\| (AC006201) histoneb H1 [Arabidopsis | 277 |
| | AT | thaliana] | |
| | | -AAF64525.1 AF253416 Lycopersicon chilense | |
| | | histone H1 variant. | |
| | | -AAB03076.1 U01890 Lycopersicon pennellii: histone | |
| | | H1. | |
| 57 | | | 91 |
| 63 | 15132_S_{—} | : gb\|AAD30449.1\|AF121878_1 (AF121878) cytidine | 323 |
| | AT | deaminase [Arabidopsis thaliana] | |
| 67 | 12656_AT | : gb\|AAD21751.1\| (AC006569) unknown protein | 283 |
| | | [Arabidopsis thaliana] | |
| 71 | 12096_AT | : gb\|AAD20908.1\| (AC006234) unknown protein | |
| | | [Arabidopsis thaliana] | |
| 77 | 19386_AT | : No hits found less than or equal to 1e-15. | |
| | | -BAB41080.1 AB052729 Pisum sativum DNA- | |
| | | binding protein DF1.DF1 | |
| 79 | 17237_AT | : gb\|AAC32431.1\| (AC004786) unknown protein | 241 |
| | | [Arabidopsis thaliana] | |
| 79 | | | 5 |
| 87 | 12431_AT | : gb\|AAC31222.1\| (AC004747) putative heat shock | 35 |
| | | transcription factor [Arabidopsis thaliana] | |
| | | -CAA47870.1 X67601 Lycopersicon peruvianum | |
| | | heat stress transcription factor HSF30. hsf30. | |
| | | -CAA87076.1 Z46952 Glycine max heat shock | |
| | | transcription factor 21. HSF. | |
| 87 | | | 197 |
| 89 | 20491_AT | : gb\|AAC95203.1\| (AC004561) putative tropinone | 193 |
| | | reductase [Arabidopsis thaliana] | |
| | | -AAA33280.1 L20475 Datura stramonium 29kDa | |
| | | protein; high homology to aa sequence of tropinone | |
| | | reductases. | |
| | | -AAA33281.1 L20473 Datura stramonium catalyses | |
| | | a stereospecific reduction oftropinone to tropine. tropinone | |
| | | reductase-I. | |
| 89 | | | 127 |
| 93 | 15792_AT | : gb\|AAB67625.1\| (AC002341) hypothetical protein | 279 |
| | | [Arabidopsis thaliana] | |
| | | -BAA94212.1 AP001633 Oryza sativa Similar to | |
| | | Arabidopsis thaliana chromosome 1 BACF9K20 genomic | |
| | | sequence; F9K20.25 (AC005679). | |
| 93 | | | 77 |
| 95 | 14856_S_{—} | : gb\|AAC26691.1\| (AC004077) putative cytochrome | 233 |
| | AT | P450 [Arabidopsis thaliana] | |
| 97 | 17051_S_{—} | : gb\|AAD09952.1\| (AF098947) CTF2B [Arabidopsis | 287 |
| | AT | thaliana] | |
| 97 | | | 53 |
| 99 | 19051_AT | : gb\|AAD31582.1\|AC006922_14 (AC006922) putative | 135 |
| | | glucosyltransferase [Arabidopsis thaliana] | |
| 99 | | | 305 |
| 101 | 15982_S_{—} | : emb\|CAA66863.1\| (X98190) peroxidase ATP2a | 313 |
| | AT | [Arabidopsis thaliana] | |
| | | -BAB16317.1 AB049589 Avicennia marina secretory | |
| | | peroxidase. PER. | |
| | | -AAD37374.1 AF145348 Glycine max peroxidase. | |
| | | Prx2b | |
| 101 | | | 61 |
| 103 | 16461_I_{—} | : gb\|AAC28766.1\| (AC004683) peroxidase [Arabidopsis | 17 |
| | AT | thaliana] | |
| | 16462_S_{—} | -BAA14144.1 D90116 Armoracia rusticana: | |
| | AT | peroxidase isozyme. | |
| | | -AAA33377.1 M37156 Armoracia rusticana: | |
| | | HRPC1. | |
| 105 | 16963_AT | : gb\|AAC28765.1\| (AC004683) peroxidase [Arabidopsis | |
| | | thaliana] | |
| | | -BAA14144.1 D90116 Armoracia rusticana: | |
| | | peroxidase isozyme. | |
| | | -AAA33377.1 M37156 Armoracia rusticana: | |
| | | HRPC1. | |
| 109 | 17379_AT | : gb\|AAF18728.1\|AC018721_3 (AC018721) putative | 19 |
| | | CCCH-type zinc finger protein [Arabidopsis thaliana] | |
| 109 | | | 199 |
| 113 | 19363_AT | : gb\|AAD22993.1\|AC007087_12 (AC007087) unknown | 217 |
| | | protein [Arabidopsis thaliana] | |
| 113 | | | 117 |
| 115 | 20174_S_{—} | : gb\|AAD22125.1\|AC006224_7 (AC006224) unknown | 335 |
| | AT | protein [Arabidopsis thaliana] | |
| 117 | 19171_AT | : gb\|AAB64325.1\| (AC002335) putative trypsin inhibitor | |
| | | [Arabidopsis thaliana] | |
| | | -CAA58994.1 X84208 Sinapis alba trypsin inhibitor | |
| | | 2. mti-2. | |
| 119 | 17840_S_{—} | : gb\|AAB64049.1\| (AC002333) putative endochitinase | |
| | AT | [Arabidopsis thaliana] | |
| | | -AAK62047.1 AY035389 Brassica napus chitinase | |
| | | class 4-like protein. | |
| | | -AAB01665.1 U21848 Brassica napus chitinase | |
| | | class IV. LSC222 | |
| 121 | 15137_S_{—} | : gb\|AAB47973.1\| (U57320) blue copper-binding protein | 217 |
| | AT | II [Arabidopsis thaliana] | |
| | | -CAC39044.1 AJ307662 Oryza sativa uclacyanin 3- | |
| | | like protein. C345ERIPDM. | |
| | | -CAA80963.1 Z25471 Pisum sativum blue copper | |
| | | protein | |
| 123 | 20269_AT | : gb\|AAB82640.1\| (AC002387) putative pectinesterase | 117 |
| | | [Arabidopsis thaliana] | |
| | | -CAB57457.2 AJ249786 Nicotiana tabacum tobacco | |
| | | mosaic virus movement protein receptor. pectin | |
| | | methylesterase. pectin methylesterase. | |
| | | -CAA69206.1 Y07899 Carica papaya de- | |
| | | esterification of cell wall pectin, pectinesterase. spe1 | |
| 127 | 19999_S_{—} | : gb\|AAD20160.1\| (AC006418) unknown protein | |
| | AT, | [Arabidopsis thaliana] | |
| | 14094_S_{—} | | |
| | AT | | |
| 133 | 12332_S_{—} | : dbj\|BAA82810.1\| (AB023448) basic endochitinase | 225 |
| | AT, | [Arabidopsis thaliana] | |
| | 13211_S_{—} | -BAA82826.1 AB023464 Arabis gemmifera basic | |
| | AT | endochitinase. ChiB. | |
| | | -AAF69793.1 AF135153 Arabis parishii class I | |
| | | chitinase. | |
| 133 | | | 67 |
| 135 | 20239_G_{—} | : emb\|CAA52619.1\| (X74514) beta-fructofuranosidase | 333 |
| | AT, | [Arabidopsis thaliana] | |
| | 20238_AT | -AAA03516.1 M58362 Daucus carota beta- | |
| | | fructosidase. cell wall. | |
| | | -CAA79676.1 Z21486 Solanum tuberosum cleavage | |
| | | of sucrose to glucose and fructose. beta-fructofuranosidase. | |
| 137 | 14248_AT | : gb\|AAD31062.1\|AC007357_11 (AC007357) Strong | 31 |
| | | similarity to gb\|X97864 cytochrome P450 from A. thaliana | |
| | | -AAL24049.1 AF426451 Citrus sinensis cytochrome | |
| | | P450. | |
| | | -AAC39318.1 AF029858 Sorghum bicolor second | |
| | | multifunctional cytochrome P450 in the biosynthetic | |
| | | pathway of the cyanogenic glucoside dhurrin. Catalyzes the | |
| | | conversion of p-hydroxyphenylacetaldoxime top- | |
| | | hydroxymandelonitrile. cytochrome P450 CYP71E1. | |
| | | CYP71E1. No EST#s identified | |
| 137 | | | 181 |
| 139 | 15969_S_{—} | : emb\|CAB37193.1\| (AJ133036) peroxidase [Arabidopsis | 167 |
| | AT | thaliana] | |
| | | -AAA33378.1 M37157 Armoracia rusticana: | |
| | | HRPC2. | |
| | | -AAA33377.1 M37156 Armoracia rusticana: HRPC1 | |
| 139 | | | 59 |
| 143 | 13219_S_{—} | : emb\|CAA74930.1\| (Y14590) class IV chitinase | 211 |
| | AT | [Arabidopsis thaliana] | |
| | | -CAA40474.1 X57187 Phaseolus vulgaris chitinase. | |
| | | Chi4. | |
| | | -AAB65776.1 U97521 Vitis vinifera class IV | |
| | | endochitinase. VvChi4A | |
| 143 | | | 71 |
| 149 | 15924_AT | : gb\|AAD22658.1\|AC007138_22 (AC007138) predicted | 155 |
| | | protein of unknown function [Arabidopsis thaliana] | |
| 155 | 20716_AT | : “gb\|AAC35547.1\| (AF080120) contains similarity to | 179 |
| | | protein kinases (Pfam: pkinase.hmm, score: 24.94) | |
| | | [Arabidopsis thaliana]” | |
| | | -AAG31173.1 AF315714 Ipomoea nil COP1. | |
| | | -CAB89693.1 AJ276591 Pisum sativum represser of | |
| | | photomorphogenesis. constitutively photomorphogenic 1 | |
| | | protein. cop1 | |
| 157 | 12748_F_{—} | : emb\|CAB51416.1\| (AL096882) drought-inducible | 183 |
| | AT | cysteine proteinase RD21A precursor-like protein | |
| | | [A.thaliana] | |
| | | -CAB17076.1 Z99954 Phaseolus vulgaris protein | |
| | | hydrolysis. cysteine proteinase precursor. | |
| | | -CAA05894.1 AJ003137 Lycopersicon esculentum: | |
| | | cysteine protease. CYP1. C14 | |
| 157 | | | 107 |
| 159 | 16914_S_{—} | : emb\|CAB39936.1\| (AL049500) osmotin precursor | 229 |
| | AT | [Arabidopsis thaliana] | |
| | | -CAA09228.1 AJ010501 Cicer arietinum thaumatin- | |
| | | like protein PR-5b. | |
| | | -AAD55090.1 AF178653 Vitis riparia thaumatin. | |
| | | osmotin; pathogenesis-related protein. | |
| 159 | | | 41 |
| 161 | 14598_AT | : emb\|CAB45970.1\| (AL080318) putative protein | |
| | | [Arabidopsis thaliana] | |
| 163 | 17963_AT | : emb\|CAB41717.1\| (AL049730) pEARLI 1-like protein | 137 |
| | | [Arabidopsis thaliana] | |
| | | -AAD01800.1 AF026382 Fragaria x ananassa: | |
| | | HyPRP. proline-rich protein. | |
| | | -BAA11855.1 D83227 Populus nigra extensin like | |
| | | protein. | |
| 165 | 16150_S_{—} | : emb\|CAB41718.1\| (AL049730) pEARLI 1 [Arabidopsis | 25 |
| | AT | thaliana] | |
| | | -AAD01800.1 AF026382 Fragaria x ananassa: | |
| | | HyPRP. proline-rich protein. | |
| | | -AAC60566.1 S68113 Brassica napus proline-rich | |
| | | SAC51. This sequence comes from FIG. 3. | |
| 165 | | | 257 |
| 173 | 20421_AT | : emb\|CAB10242.1\| (Z97336) germin precursor oxalate | 235 |
| | | oxidase [Arabidopsis thaliana] | |
| | | -CAB55394.1 AL117264 Oryza sativa zwh0010.1. | |
| | | similar to Arabidopsis germin-like protein 6(AF032976); | |
| | | Method: conceptual translation with partialpeptide | |
| | | sequencing. | |
| | | -BAA78563.1 AB024338 Atriplex lentiformis: germin- | |
| | | like protein. | |
| 173 | | | 1 |
| 175 | 17899_AT | : emb\|CAB10339.1\| (Z97339) hypothetical protein | 341 |
| | | [Arabidopsis thaliana] | |
| 177 | 17485_S_{—} | : “emb\|CAB10405.1\| (Z97340) beta-1, 3-glucanase class I | 37 |
| | AT | precursor [Arabidopsis thaliana]” | |
| | | -CAB38443.1 AJ133470 Hevea brasiliensis: beta-1,3- | |
| | | glucanase. hgn1. | |
| | | -AAA87456.1 U22147 Hevea brasiliensis: beta-1,3- | |
| | | glucanase. HGN1. hydrolytic enzyme. | |
| 177 | | | 321 |
| 185 | 19850_AT | : emb\|CAA23068.1\| (AL035396) putative protein | 227 |
| | | [Arabidopsis thaliana] | |
| 187 | 12815_AT | : emb\|CAB43877.1\| (AL078467) putative protein | 293 |
| | | [Arabidopsis thaliana] | |
| 187 | | | 103 |
| 189 | 16003_S_{—} | : “emb\|CAA16877.1\| (AL021749) ADP, ATP carrier-like | 191 |
| | AT | protein [Arabidopsis thaliana]” | |
| | | -CAA41812.1 X59086 Zea mays adenine | |
| | | nucleotide translocator. MANT2. | |
| | | -AAB72047.1 AF006489 Gossypium hirsutum | |
| | | adenine nucleotide translocator 1. CANT1 | |
| 189 | | | 27 |
| 193 | 19182_AT | : emb\|CAA21214.1\| (AL031804) putative protein | 307 |
| | | [Arabidopsis thaliana] | |
| 195 | 17920_S_{—} | : emb\|CAA21216.1\| (AL031804) pyruvate | 223 |
| | AT | decarboxylase-1 (Pdc1) [Arabidopsis thaliana] | |
| | | -AAL37492.1 AF333772 Fragaria x ananassa: | |
| | | pyruvate decarboxylase. pdc1. | |
| | | -AAG13131.1 AF193791 Fragaria x ananassa: | |
| | | pyruvate decarboxylase. PDC | |
| 195 | | | 95 |
| 197 | 12933_R_{—} | : emb\|CAA65420.1\| (X96600) pathogenesis-related | 177 |
| | AT | protein 1 [Arabidopsis thaliana] | |
| | | -CAA30017.1 X06930 Nicotiana tabacum PR-1a | |
| | | protein (AA 1-168). | |
| | | -CAA31010.1 X12487 Nicotiana tabacum PR1c | |
| | | preprotein | |
| 197 | | | 23 |
| 199 | 20308_S_{—} | : “emb\|CAA20585.1\| (AL031394) pathogenesis-related | 85 |
| | AT | protein 1 precursor, 19.3K [Arabidopsis thaliana]” | |
| | | -CAA47374.1 X66942 Nicotiana tabacum prb-1b. | |
| | | PRB-1B. | |
| | | -AAK30143.1 AF348141 Capsicum annuum | |
| | | pathogenesis-related protein PR-1 precursor. | |
| 199 | | | 203 |
| 201 | 12070_S_{—} | : emb\|CAA18845.1] (AL023094) putative protein | 153 |
| | AT | [Arabidopsis thaliana] | |
| 201 | | | 143 |
| 203 | 19846_AT | : emb\|CAA18742.1\| (AL022604) putative protein | 255 |
| | | [Arabidopsis thaliana] | |
| | | -CAB61243.1 AJ239041 Lotus japonicus nodule | |
| | | organogenesis. nodule inception protein. nin. | |
| 209 | 17930_S_{—} | : emb\|CAA07352.1\| (AJ006960) peroxidase [Arabidopsis | 185 |
| | AT | thaliana] | |
| | | -CAA09881.1 AJ011939 Trifolium repens peroxidase. | |
| | | prx2. | |
| 211 | 16514_AT | : emb\|CAB37548.1\| (AL035538) putative protein | |
| | | [Arabidopsis thaliana] | |
| 213 | 19845_G_{—} | : emb\|CAB37510.1\| (AL035540) monooxygenase 2 | |
| | AT | (MO2) | |
| | | [Arabidopsis thaliana] | |
| 221 | 16916_S_{—} | : emb\|CAA54420.1\| (X77199) heat shock cognate 70-2 | 213 |
| | AT | [Arabidopsis thaliana] | |
| | | -AAB88009.1 AF035414 Brassica napus heat shock | |
| | | cognate protein HSC70. Hsc70. | |
| | | -CAA47948.1 X67711 Oryza sativa heat shock | |
| | | protein 70. hsp70 | |
| 221 | | | 97 |
| 227 | 17589_AT | : gb\|AAF00612.1\|AF156783_1 (AF156783) apyrase | 285 |
| | | [Arabidopsis thaliana] | |
| | | -AAG22044.1 AF305783 Pisum sativum apyrase 2. | |
| | | apy2. phosphatase. | |
| | | -AAF00610.1 AF156781 Dolichos biflorus apyrase. | |
| | | apyrase-2 | |
| 227 | | | 73 |
| 231 | 15125_F_{—} | : dbj\|BAA22095.1\| (D85190) vegetative storage protein | 231 |
| | AT | [Arabidopsis thaliana] | |
| | | -CAA11075.1 AJ223074 Glycine max acid | |
| | | phosphatase. | |
| | | -AAA33937.1 M37530 Glycine max 28 kDa | |
| | | protein | |
| 231 | | | 33 |
| 239 | 14621_AT | : gb\|AAC31244.1\| (AC004747) putative antifungal | |
| | | protein [Arabidopsis thaliana] | |
| | | -AAA69541.1 U18557 Raphanus sativus antifungal, | |
| | | fungistatic. antifungal protein 1 preprotein. Rs-AFP1. | |
| | | -BAB19054.1 AB012871 Wasabia japonica gamma- | |
| | | thionin1. | |
| 241 | 16091_S_{—} | : gb\|AAA32822.1\| (M62984) heat shock protein 83 | |
| | AT, | [Arabidopsis thaliana] | |
| | 13285_S_{—} | -AAA33748.1 M99431 Ipomoea nil heat shock | |
| | AT | protein 83. Hsp83A. | |
| | | -AAB26482.2 S59780 Zea mays heat shock protein | |
| | | HSP82. hsp82. 82 kda; This sequence comes from FIG. 3; | |
| | | conceptual translation differs from published | |
| | | sequence;mismatches(66[K->N],67[L->V],68[D->N]). | |
| 245 | 12876_S_{—} | : gb\|AAA97403.1\| (U33473) AGL8 [Arabidopsis | 303 |
| | AT | thaliana] | |
| | | -AAB41525.1 U25695 Sinapis alba transcription | |
| | | factor SaMADS B. MADS box protein. | |
| | | -CAA67969.1 X99655 Betula pendula MADS5 | |
| | | protein. MADS5 | |
| 247 | 15985_AT | : emb\|CAA67340.1\| (X98808) peroxidase ATP3a | 319 |
| | | [Arabidopsis thaliana] | |
| | | -CAA64413.1 X94943 Lycopersicon esculentum: | |
| | | peroxidase. cevi16. | |
| | | -AAA32676.1 M37637 Arachis hypogaea cationic | |
| | | peroxidase. PNC2. | |
| 247 | | | 15 |
| 249 | 20320_AT | : gb[AAC00628.1\| (AC002291) Similar to ‘MADS box’ | 161 |
| | | transcription factors [Arabidopsis thaliana] | |
| | | -CAB97354.1 AJ249146 Hordeum vulgare MADS- | |
| | | box protein 8. m8. | |
| | | -AAG43200.1 AF112150 Zea mays MADS box | |
| | | protein 3. mads3 | |
| 249 | | | 45 |

### Example 3

### Rice Orthologs of Arabidopsis PTGS Genes Identified by Reverse Genetics

Understanding the function of every gene is the major challenge in the age of completely sequenced eukaryotic genomes. Sequence homology can be helpful in identifying possible functions of many genes. However, reverse genetics, the process of identifying the function of a gene by obtaining and studying the phenotype of an individual containing a mutation in that gene, is another approach to identify the function of a gene.

Reverse genetics in Arabidopsis has been aided by the establishment of large publicly available collections of insertion mutants (Krysan et al., (1999) Plant Cell 11, 2283-2290; Tisser et al., (1999) Plant Cell 11, 1841-1852; Speulman et al., (1999). Plant Cell 11, 1853-1866; Parinov et al., (1999). Plant Cell 11, 2263-2270; Parinov and Sundaresan, 2000; Biotechnology 11, 157-161). Mutations in genes of interest are identified by screening the population by PCR amplification using primers derived from sequences near the insert border and the gene of interest to screen through large pools of individuals. Pools producing PCR products are confirmed by Southern hybridization and further deconvoluted into subpools until the individual is identified (Sussman et al., (2000) Plant Physiology 124, 1465-1467).

Recently, some groups have begun the process of sequencing insertion site flanking regions from individual plants in large insertion mutant populations, in effect prescreening a subset of lines for genomic insertion sites (Parinov et al., (1999). Plant Cell 11, 2263-2270; Tisser et al., (1999). Plant Cell 11, 1841-1852). The advantage to this approach is that the laborious and time-consuming process of PCR-based screening and deconvolution of pools is avoided.

A large database of insertion site flanking sequences from approximately 100,000 T-DNA mutagenized Arabidopsis plants of the Columbia ecotype (GARLIC lines) is prepared. T-DNA left border sequences from individual plants are amplified using a modified thermal asymmetric interlaced-polymerase chain reaction (TAIL-PCR) protocol (Liu et al., (1995). Plant J. 8, 457-463). Left border TAIL-PCR products are sequenced and assembled into a database that associates sequence tags with each of the approximately 100,000 plants in the mutant collection. Screening the collection for insertions in genes of interest involves a simple gene name or sequence BLAST query of the insertion site flanking sequence database, and search results point to individual lines. Insertions are confirmed using PCR.

Analysis of the GARLIC insert lines suggests that there are 76,856 insertions that localize to a subset of the genome representing coding regions and promoters of 22,880 genes. Of these, 49,231 insertions lie in the promoters of over 18,572 genes, and an additional 27,625 insertions are located within the coding regions of 13,612 genes. Approximately 25,000 T-DNA left border mTAIL-PCR products (25% of the total 102,765) do not have significant matches to the subset of the genome representing promoters and coding regions, and are therefore presumed to lie in noncoding and/or repetitive regions of the genome.

The Arabidopsis T-DNA GARLIC insertion collection is used to investigate the roles of certain genes involved in posttranscriptional gene silencing (“PTGS”). Target genes are chosen using a variety of criteria, including public reports of mutant phenotypes, RNA profiling experiments, and sequence similarity to genes implicated in PTGS. Plant lines with insertions in genes of interest are then identified. Each T-DNA insertion line is represented by a seed lot collected from a plant that is hemizygous for a particular T-DNA insertion. Plants homozygous for insertions of interest are identified using a PCR assay. The seed produced by these plants is homozygous for the T-DNA insertion mutation of interest.

Homozygous mutant plants are tested for altered posttranscriptional silencing behavior. The genes interrupted in these mutants contribute to the observed phenotype. The genes interrupted in these mutants interfere with the normal silencing status of the plant.

Rice orthologs of the Arabidopsis genes involved in posttranscriptional gene silencing are identified by similarity searching of a rice database using the Double-Affine Smith-Waterman algorithm (BLASP with e values better than ⁻¹⁰).

### Example 4

### Cloning and Sequencing of Nucleic Acid Molecules from Rice

4.1 Genomic DNA: Plant genomic DNA samples are isolated from a collection of tissues. Individual tissues are collected from a minimum of five plants and pooled. DNA can be isolated according to one of the three procedures, e.g., standard procedures described by Ausubel et al. (1995), a quick leaf prep described by Klimyuk et al. (1993), or using FTA paper (Life Technologies).

For the latter procedure, a piece of plant tissue such as, for example, leaf tissue is excised from the plant, placed on top of the FTA paper and covered with a small piece of parafilm that serves as a barrier material to prevent contamination of the crushing device. In order to drive the sap and cells from the plant tissue into the FTA paper matrix for effective cell lysis and nucleic acid entrapment, a crushing device is used to mash the tissue into the FTA paper. The FTA paper is air dried for an hour. For analysis of DNA, the samples can be archived on the paper until analysis. Two mm punches are removed from the specimen area on the FTA paper using a 2 mm Harris Micro Punch and placed into PCR tubes. Two hundred (200) microliters of FTA purification reagent is added to the tube containing the punch and vortexed at low speed for 2 seconds. The tube is then incubated at room temperature for 5 minutes. The solution is removed with a pipette so as to repeat the wash one more time. Two hundred (200) microliters of TE (10 mM Tris, 0.1 mM EDTA, pH 8.0) is added and the wash is repeated two more times. The PCR mix is added directly to the punch for subsequent PCR reactions.

4.2 Cloning of Candidate cDNA: A candidate cDNA is amplified from total RNA isolated from rice tissue after reverse transcription using primers designed against the computationally predicted cDNA. Primers designed based on the genomic sequence can be used to PCR amplify the full-length CDNA (start to stop codon) from first strand cDNA prepared from rice cultivar Nipponbare tissue.

The Qiagen RNeasy kit (Qiagen, Hilden, Germany) is used for extraction of total RNA. The Superscript II kit (Invitrogen, Carlsbad, USA) is used for the reverse transcription reaction. PCR amplification of the candidate CDNA is carried out using the reverse primer sequence located at the translation start of the candidate gene in 5′-3′ direction. This is performed with high-fidelity Taq polymerase (Invitrogen, Carlsbad, USA).

The PCR fragment is then cloned into pCR2.1-TOPO (Invitrogen) or the pGEM-T easy vector (Promega Corporation, Madison, Wis., USA) per the manufacturer's instructions, and several individual clones are subjected to sequencing analysis.

4.3 DNA sequencing: DNA preps for 2-4 independent clones are miniprepped following the manufacturer's instructions (Qiagen). DNA is subjected to sequencing analysis using the BigDye™ Terminator Kit according to manufacturer's instructions (ABI). Sequencing makes use of primers designed to both strands of the predicted gene of interest. DNA sequencing is performed using standard dye-terminator sequencing procedures and automated sequencers (models 373 and 377; Applied Biosystems, Foster City, Calif.). All sequencing data are analyzed and assembled using the Phred/Phrap/Consed software package (University of Washington) to an error ratio equal to or less than 10⁻⁴ at the consensus sequence level.

The consensus sequence from the sequencing analysis is then to be validated as being intact and the correct gene in several ways. The coding region is checked for being full length (predicted start and stop codons present) and uninterrupted (no internal stop codons). Alignment with the gene prediction and BLAST analysis is used to ascertain that this is in fact the right gene.

The clones are sequenced to verify their correct amplification.

### Example 5

### Functional Analysis in Plants

### Example 5.1

### Plant Complementation Assay

A plant complementation assay can be used for the functional characterization of the PTGS genes according to the invention.

Rice and Arabidopsis putative orthologue pairs are identified using BLAST comparisons, TFASTXY comparisons, and Double-Affine Smith-Waterman similarity searches. Constructs containing a rice cDNA or genomic clone inserted between the promoter and terminator of the Arabidopsis orthologue are generated using overlap PCR (Gene 77, 61-68 (1989)) and GATEWAY cloning (Life Technologies Invitrogen). For ease of cloning, rice cDNA clones are preferred to rice genomic clones. A three stage PCR strategy is used to make these constructs.

(1) In the first stage, primers are used to PCR amplify: (i) 2 Kb upstream of the translation start site of the Arabidopsis orthologue, (ii) the coding region or cDNA of the rice orthologue, and (iii) the 500 bp immediately downstream of the Arabidopsis orthogue's translation stop site. Primers are designed to incorporate onto their 5′ ends at least 16 bases of the 3′ end of the adjacent fragment, except in the case of the most distal primers which flank the gene construct (the forward primer of the promoter and the reverse primer of the terminator). The forward primer of the promoters contains on their 5′ ends partial AttB1 sites, and the reverse primer of the terminators contains on their 5′ ends partial AttB2 sites, for Gateway cloning.

(2) In the second stage, overlap PCR is used to join either the promoter and the coding region, or the coding region and the terminator.

(3) In the third stage either the promoter-coding region product can be joined to the terminator or the coding region-terminator product can be joined to the promoter, using overlap PCR and amplification with fulll Att site-containing primers, to link all three fragments, and put full Att sites at the construct termini.

The fused three-fragment piece flanked by Gateway cloning sites are introduced into the LTI donor vector pDONR201 (Invitrogen) using the BP clonase reaction, for confirmation by sequencing. Confirmed sequenced constructs are introduced into a binary vector containing Gateway cloning sites, using the LR clonase reaction such as, for example, pAS200.

The pAS200 vector was created by inserting the Gateway cloning cassette RfA into the Acc65I site of pNOV3510.

pNOV3510 was created by ligation of inverted pNOV2114 VSI binary into pNOV3507, a vector containing a PTX5′ Arab Protox promoter driving the PPO gene with the Nos terminator.

pNOV2114 was created by insertion of virGN54D (Pazour et al. 1992, J. Bacteriol. 174:4169-4174) from pAD1289 (Hansen et al. 1994, PNAS 91:7603-7607) into pHiNK085.

pHiNK085 was created by deleting the 35S:PMI cassette and M13 ori in pVictorHiNK.

pPVictorHiNK was created by modifying the T-DNA of pVictor (described in WO 97/04112) to delete M13 derived sequences and to improve its cloning versatility by introducing the BIGLINK polylinker.

The sequence of the pVictor HiNK vector is disclosed in SEQ ID NO: 5 in WO 00/6837, which is incorporated herein by reference. The pVictorHiNK vector contains the following constituents that are of functional importance:

The origin of replication (ORI) functional in Agrobacterium is derived from the Pseudomonas aeruginosa plasmid pVS1 (Itoh et al 1984. Plasmid 11: 206-220; Itoh and Haas, 1985. Gene 36: 27-36). The pVS1 ORI is only functional in Agrobacterium and can be mobilised by the helper plasmid pRK2013 from E. coli into A. tumefaciens by means of a triparental mating procedure (Ditta et al., 1980. Proc. Natl. Acad. Sci USA 77: 7347-7351).

The ColE1 origin of replication functional in E. coli is derived from pUC 19 (Yannisch-Perron et al., 1985. Gene 33: 103-119).

The bacterial resistance to spectinomycin and streptomycin encoded by a 0.93 kb fragment from transposon Tn7 (Fling et al., 1985. Nucl. Acids Res. 13: 7095) functions as selectable marker for maintenance of the vector in E. coli and Agrobacterium. The gene is fused to the tac promoter for efficient bacterial expression (Amman et al., 1983. Gene 25: 167-178).

The right and left T-DNA border fragments of 1.9 kb and 0.9 kb that comprise the 24 bp border repeats, have been derived from the Ti-plasmid of the nopaline type Agrobacterium tumefaciens strains pTiT37 (Yadav et al., 1982. Proc. Natl. Acad. Sci. USA. 79: 6322-6326).

The plasmid is introduced into Agrobacterium tumefaciens GV3101pMP90 by electroporation. The positive bacterial transformants are selected on LB medium containing 50 μg/μl kanamycin and 25 μg/μl gentamycin. Plants are transformed by standard methodology (e.g., by dipping flowers into a solution containing the Agrobacterium) except that 0.02% Silwet -77 (Lehle Seeds, Round Rock, Tex.) is added to the bacterial suspension and the vacuum step omitted. Five hundred (500) mg of seeds are planted per 2 ft² flat of soil and, and progeny seeds are selected for transformants using PPO selection.

Primary transformants are analyzed for complementation. Primary transformants are genotyped for the Arabidopsis mutation and presence of the transgene. When possible, >50 mutants harboring the transgene should be phenotyped to observe variation due to transgene copy number and expression

### Example 5.2

### Complementation of a PTGS Deficiency of an Arabidopsis T-DNA Insertion Line by Overexpression of a Nucleotide Sequence Encoding a Polypeptide as Given in SEQ ID NOs: 328 and 317, Respectively to Confirm that a Polypeptide is Required for PTGS

A construction designed to overexpress a polypeptide according to SEQ ID NOs: 328 and 317, respectively is introduced into Arabidopsis plants, which have a T-DNA insertion in the respective genes encoding those proteins. The coding sequence is amplified by RT-PCR from RNA prepared from Arabidopsis leaves using appropriate primers. This coding sequence is then placed under the regulation of the strong, constitutive UBQ3 gene promoter (BAC F15A17, GenBank accession AL163002) in binary vector pCAMBIA-1380 (GenBank accession AF234301). The resultant expression vector is designated pCAND1.

For complementation studies, CAND1 transformants obtained by transformation of wild-type Arabidopsis plants with the vector pCAND1 are allowed to self-fertilize. The resultant T1 generation plants are tested for the hygromycin resistance phenotype to detect the presence of the CANDI T-DNA. The hygromycin-resistant plants are then allowed to self fertilize and the resultant T2 generation is scored for hygromycin-resistance to identify homozygous transformants with T-DNA inserts at a single locus.

Homozygous CAND1 transformants are then crossed with PTGS lines 8Z-2 and 5 to obtain F3 generation plants homozygous for both the 35S-GFP and CAND1 transgenes by using the methods described herein previously. By assaying these homozygous lines for GFP expression as described an increase in the fraction of plants exhibiting PTGS among the 8Z-2 CAND and 5 CAND plants may be detected compared with the original 8Z-2 and 5 lines, respectively.

### Example 6

### Insertion Mutagenesis in Rice

Example 6.1

### Insertion Mutagenesis in a Nucleotide Sequence Encoding a Transcription Factor Polypeptide in Rice

As described above in Examples 5.1 and 5.2, insertional mutatgeneis is used to direct reverse genetic screens. To assess the function of a polypeptide encoded by the nucleotide sequence set forth in SEQ ID NO: 328, a pool of independent tagged rice lines is screened by PCR utilizing pairs of primers corresponding to the T-DNA left border or retro-transposon tag Tos 17 and the SEQ ID NO: 328 3′-specific region. For T-DNA tagged line, the left border primer from A. tumefaciens T-DNA vector pD991 is represented by SEQ ID NO: 781 (5′-cat ttt ata ata acg ctg cgg aca tct ac -3′). (Krysan et al., 1999). A specific PCR product is identified and isolated. Sequencing of the PCR-amplified fragment reveals a T-DNA insertion in the predicted CDS region.

### Example 6.2

### Rice transgenic Line Exhibiting Post-transcriptional Silencing of a Green-fluorescent Protein Reporter Gene

Agrobacterium-mediated transformation methods known in the art and described in Example 8.1 below is used to obtain transgenic rice plants exhibiting PTGS. The Ti-plasmid used contains a chimeric green fluorescent protein (GFP) (Reichel et al. (1996) PNAS 93: 5888-93) reporter gene regulated by a duplicated cauliflower mosaic virus (CaMV) 35S RNA promoter and transcriptional terminator (Goodall and Filipowicz (1989) Cell 58: 473-483) in the binary vector pBIN19 (Bevan (1984) Nucl. Acids Res. 12: 8711-8721). The T-DNA region of this plasmid (p35S-GFP) is shown schematically in FIG. 1.

To evaluate PTGS in the resultant 35S-GFP transformants, GFP expression is monitored in transgenic plants by GFP excitation with UV light (approximate range of wavelengths 390 to 480 nm). Selection of transgenic lines showing PTGS is based on absence of GFP expression in mature plants that showed normal GFP expression in earlier stages of plant development. Based on this criterion, lines which are homozygous for the T-DNA insert, show PTGS associated with greatly reduced GFP-mRNA levels detected by RNA blot hybridization as described by Sambrook et al. (Molecular Cloning, 2^{nd} edition. 1989).

DNA blot hybridization as described by Sambrook et al. (Molecular Cloning. 2^{nd} edition, 1989) is used to determine the position of the T-DNA inserts.

### Example 6.3

### Analysis of the Expression of a Characterized Silenced Transgene in Rice Lines

The Rice line obtained in Example 6.1 is crossed with the line from Example 6.2 and the resultant F1 generation plants are allowed to self-fertilize to obtain the F2 generation. F2 plants are grown and tested for a presence of T-DNA insertion in the PTGS gene derived from the parental line and for the 35S-GFP T-DNA derived from the other parental line. The presence of the T-DNA insertion in the PTGS gene is demonstrated as described in Example 2. Plants homozygous for this T-DNA insertion are then checked for homozygosity by PCR using a 3′ specific primer and 36851TD#3 (5′-gct ccg ccc aca taa ttc aaa caa cac-3′, SEQ ID NO 782). These primers span a region of genomic DNA including the insertion site such that only the wild-type copy of DNA results in amplification of a genomic fragment. A similar strategy is used to screen for lines homozygous for the 35S-GFP T-DNA. First, the presence of the 35S-GFP T-DNA is demonstrated by using the T-DNA-specific PCR primer LB1 and the gene-specific PCR primer. Second, plants carrying the 35S-GFP T-DNA are tested for homozygosity by using the gene-specific primers. The plants homozygous for both the transgenes are allowed to self fertilize to obtain F3 generation plants. These plants and the parental line are scored for incidence of PTGS based on GFP fluorescence as described in Example 8.

### Example 7

### Vector Construction for Overexpression and Gene “Knockout” Experiments

7.1 Overexpression

Vectors used for expression of full-length “PTGS candidate genes” of interest in plants (overexpression) are designed to overexpress the protein of interest and are of two general types, biolistic and binary, depending on the plant transformation method to be used.

For biolistic transformation (biolistic vectors), the requirements are as follows:

1. a backbone with a bacterial selectable marker (typically, an antibiotic resistance gene) and origin of replication functional in Escherichia coli (E. coli; eg. ColE1), and

2. a plant-specific portion consisting of:

a. a gene expression cassette consisting of a promoter (eg. ZmUBlint MOD), the gene of interest (typically, a full-length cDNA) and a transcriptional terminator (eg. Agrobacterium tumefaciens nos terminator);

b. a plant selectable marker cassette, consisting of a promoter (eg. rice Act1D-BV MOD), selectable marker gene (eg. phosphomannose isomerase, PMI) and transcriptional terminator (eg. CaMV terminator).

Vectors designed for transformation by Agrobacterium tumefaciens (A. tumefaciens; binary vectors) consist of:

1. a backbone with a bacterial selectable marker functional in both E. coli and A. tumefaciens (eg. spectinomycin resistance mediated by the aadA gene) and two origins of replication, functional in each of aforementioned bacterial hosts, plus the A. tumefaciens virG gene;

2. a plant-specific portion as described for biolistic vectors above, except in this instance this portion is flanked by A. tumefaciens right and left border sequences which mediate transfer of the DNA flanked by these two sequences to the plant.

7.2 Knock Out Vectors

Vectors designed for reducing or abolishing expression of a single gene or of a family or related genes (knockout vectors) are also of two general types corresponding to the methodology used to downregulate gene expression: antisense or double-stranded RNA interference (dsRNAi).

(a) Anti-sense

For antisense vectors, a full-length or partial gene fragment (typically, a portion of the cDNA) can be used in the same vectors described for full-length expression, as part of the gene expression cassette. For antisense-mediated down-regulation of gene expression, the coding region of the gene or gene fragment will be in the opposite orientation relative to the promoter; thus, mRNA will be made from the non-coding (antisense) strand in planta.

(b) dsRNAi

For dsRNAi vectors, a partial gene fragment (typically, 300 to 500 basepairs long) is used in the gene expression cassette, and is expressed in both the sense and antisense orientations, separated by a spacer region (typically, a plant intron, eg. the OsSH1 intron 1, or a selectable marker, eg. conferring kanamycin resistance). Vectors of this type are designed to form a double-stranded mRNA stem, resulting from the basepairing of the two complementary gene fragments in planta.

Biolistic or binary vectors designed for overexpression or knockout can vary in a number of different ways, including eg. the selectable markers used in plant and bacteria, the transcriptional terminators used in the gene expression and plant selectable marker cassettes, and the methodologies used for cloning in gene or gene fragments of interest (typically, conventional restriction enzyme-mediated or Gateway™ recombinase-based cloning). An important variant is the nature of the gene expression cassette promoter driving expression of the gene or gene fragment of interest in most tissues of the plants (constitutive, eg. ZmUBlint MOD), in specific plant tissues (eg. maize ADP-gpp for endosperm-specific expression), or in an inducible fashion (eg. GAL4bsBz1 for estradiol-inducible expression in lines constitutively expressing the cognate transcriptional activator for this promoter).

7.3. Insertion of a “PTGS Candidate Gene ” into an Over-Expression Vector

A validated rice CDNA clone prepared according to Example 13.1 comprising part or all of a nucleic acid sequence as given in the Sequence Lisitng in pCR2.1-TOPO is subcloned using conventional restriction enzyme-based cloning into a vector, downstream of the maize ubiquitin promoter and intron, and upstream of the Agrobacterium tumefaciens nos 3′ end transcriptional terminator. The resultant gene expression cassette (promoter, “PTGS candidate gene” and terminator) is further subcloned, using conventional restriction enzyme-based cloning, into the pNOV2117 binary vector (Negrotto et al (2000) Plant Cell Reports 19, 798-803; plasmid pNOV117 discosed in this article corresponds to pNOV2117 described herein; ; the nucleotide sequence of pNOV2117 is provided in SEQ ID NO: 44 of WO 01/73087), generating pNOVCAND.

The pNOVCAND binary vector is designed for transformation and over-expression of the “PTGS candidate gene” in monocots. It consists of a binary backbone containing the sequences necessary for selection and growth in Escherichia coli DH-5α (Invitrogen) and Agrobacterium tumefaciens LBA4404 (pAL4404; pSB1), including the bacterial spectinomycin antibiotic resistance aadA gene from E. coli transposon Tn7, origins of replication for E. coli (ColE1) and A. tumefaciens (VS 1), and the A. tumefaciens virG gene. In addition to the binary backbone, which is identical to that of pNOV2114 described herein previously (see Example 7 above), pNOV2117 contains the T-DNA portion flanked by the right and left border sequences, and including the Positech™ (Syngenta) plant selectable marker (WO 94/20627) and the “PTGS candidate gene” gene expression cassette. The Positech™ plant selectable marker confers resistance to mannose and in this instance consists of the maize ubiquitin promoter driving expression of the PMI (phosphomannose isomerase) gene, followed by the cauliflower mosaic virus transcriptional terminator.

Plasmid pNOV2117 is introduced into Agrobacterium tumefaciens LBA4404 (pAL4404; pSB1) by electroporation. Plasmid pAL4404 is a disarmed helper plasmid (Ooms et al (1982) Plasmid 7, 15-29). Plasmid pSB1 is a plasmid with a wide host range that contains a region of homology to pNOV2117 and a 15.2 kb KpnI fragment from the virulence region of pTiBo542 (Ishida et al (1996) Nat Biotechnol 14, 745-750). Introduction of plasmid pNOV2117 into Agrobacterium strain LBA4404 results in a co-integration of pNOV2117 and pSB1.

Alternatively, plasmid pCIB7613, which contains the hygromycin phosphotransferase (hpt) gene (Gritz and Davies, Gene 25, 179-188, 1983) as a selectable marker, may be employed for transformation.

Plasmid pCIB7613 (see WO 98/06860, incorporated herein by reference in its entirety) is selected for rice transformation. In pCIB7613, the transcription of the nucleic acid sequence coding hygromycin-phosphotransferase (HYG gene) is driven by the corn ubiquitin promoter (ZmUbi) and enhanced by corn ubiquitin intron 1. The 3′polyadenylation signal is provided by NOS 3′ nontranslated region.

Other useful plasmids include pNADII002 (GAL4-ER-VP16) which contains the yeast GAL4 DNA Binding domain (Keegan et al., Science, 231:699 (1986)), the mammalian estrogen receptor ligand binding domain (Greene et al., Science, 231:1150 (1986)) and the transcriptional activation domain of the HSV VP16 protein (Triezenberg et al.,1988). Both hpt and GAL4-ER-VP16 are constitutively expressed using the maize Ubiquitin promoter, and pSGCDL1 (GAL4BS Bz1 Luciferase), which carries the firefly luciferase reporter gene under control of a minimal maize Bronze1 (Bz1) promoter with 10 upstream synthetic GAL4 binding sites. All constructs use termination signals from the nopaline synthase gene.

### Example 8

### Plant Transformation

8.1 Rice Transformation

pNOVCAND is transformed into a rice cultivar (Kaybonnet) using Agrobacterium-mediated transformation, and mannose-resistant calli are selected and regenerated.

Agrobacterium is grown on YPC solid plates for 2-3 days prior to experiment initiation. Agrobacterial colonies are suspended in liquid MS media to an OD of 0.2 at λ600 nm. Acetosyringone is added to the agrobacterial suspension to a concentration of 200 μM and agro is induced for 30 min.

Three-week-old calli which are induced from the scutellum of mature seeds in the N6 medium (Chu, C. C. et al., Sci, Sin., 18, 659-668(1975)) are incubated in the agrobacterium solution in a 100×25 petri plate for 30 minutes with occasional shaking. The solution is then removed with a pipet and the callus transfered to a MSAs medium which is overlayed with sterile filter paper.

Co-Cultivation is continued for 2 days in the dark at 22° C.

Calli are then placed on MS-Timetin plates for 1 week. After that they are transfered to PAA+ mannose selection media for 3 weeks.

Growing calli (putative events) are picked and transfered to PAA+ mannose media and cultivated for 2 weeks in light.

Colonies are tranfered to MS20SorbKinTim regeneration media in plates for 2 weeks in light. Small plantlets are transferred to MS20SorbKinTim regeneration media in GA7 containers. When they reach the lid, they are transfered to soil in the greenhouse.

Expression of the “PTGS candidate gene” in transgenic To plants is analyzed. Additional rice cultivars, such as but not limited to, Nipponbare, Taipei 309 and Fuzisaka 2 are also transformed and assayed for expression of the “PTGS candidate gene” product and enhanced protein expression.

8.2 Maize Transformation

Transformation of immature maize embryos is performed essentially as described in Negrotto et al., (2000) Plant Cell Reports 19: 798-803. For this example, all media constituents are as described in Negrotto et al., supra. However, various media constituents described in the literature may be substituted.

8.2.1 Transformation Plasmids and Selectable Marker

The genes used for transformation are cloned into a vector suitable for maize transformation as described in Example 17. Vectors used contain the phosphomannose isomerase (PMI) gene (Negrotto et al. (2000) Plant Cell Reports 19: 798-803).

8.2.2 Preparation of Agrobacterium tumefaciens

Agrobacterium strain LBA4404 (pSB1) containing the plant transformation plasmid is grown on YEP (yeast extract (5 g/L), peptone (10 g/L), NaCl (5g/L),15 g/l agar, pH 6.8) solid medium for 2 to 4 days at 28° C. Approximately 0.8X 109 Agrobacteria are suspended in LS-inf media supplemented with 100 μM acetosyringone (As) (Negrotto et al.,(2000) Plant Cell Rep 19: 798-803). Bacteria are pre-induced in this medium for 30-60 minutes.

8.2.3 Inoculation

Immature embryos from A188 or other suitable maize genotypes are excised from 8-12 day old ears into liquid LS-inf+ 100 μM As. Embryos are rinsed once with fresh infection medium. Agrobacterium solution is then added and embryos are vortexed for 30 seconds and allowed to settle with the bacteria for 5 minutes. The embryos are then transferred scutellum side up to LSAs medium and cultured in the dark for two to three days. Subsequently, between 20 and 25 embryos per petri plate are transferred to LSDc medium supplemented with cefotaxime (250 mg/l) and silver nitrate (1.6 mg/l) and cultured in the dark for 28° C. for 10 days.

8.2.4 Selection of Transformed Cells and Regeneration of Transformed Plants

Immature embryos producing embryogenic callus are transferred to LSD1M0.5S medium. The cultures are selected on this medium for 6 weeks with a subculture step at 3 weeks. Surviving calli are transferred either to LSD1M0.5S medium to be bulked-up or to Reg1 medium. Following culturing in the light (16 hour light/8 hour dark regiment), green tissues are then transferred to Reg2 medium without growth regulators and incubated for 1-2 weeks. Plantlets are transferred to Magenta GA-7 boxes (Magenta Corp, Chicago Ill.) containing Reg3 medium and grown in the light. Plants that are PCR positive for the promoter-reporter cassette are transferred to soil and grown in the greenhouse.

### Example 9

### Overexpression of a Nucleotide Sequence of a Candidate Gene Encoding a Polypeptide as Given in SEO ID NOs: 152 and 142, Respectively in Arabidopsis

A transgenic construct designed to overexpress a polypeptide according to the invention as given in SEQ ID NOs: 328 and 317, respectively, is introduced into a transgenic line comprising a second transgene. A suitable line expresses the second transgene at a high level with no silencing or without complete silencing, preferably with less than half the plants showing silencing or with the silenced plants showing silencing to levels greater than 50% of the average levels of all the plants.

The transgenic construct is created by expressing the GUS marker gene (GenBank accession S69414), using the strong constitutive ACT2 promoter (GenBank accession U41998), with the CaMV 35S transcriptional terminator (nucleotides 2868 to 2938 in pJG304 (Guyer et al., 1998, Genetics 149:633-639)) in a binary T-DNA vector. This construct is introduced into Arabidopsis via Agrobacterium-mediated transformation. T2 plants from a single T1 plant expressing high levels of GUS activity are examined for silencing.

These T2 plants, or their progeny, are also transformed with one of two constructs. One construct allows overexpression of the candidate gene with a strong promoter and a transcriptional terminator different from those used in the construct described above. The other construct is a control that is essentially the same as the candidate gene construct, except that in place of a candidate gene, a marker gene, such as luciferase or GFP is overexpressed or no gene is overexpressed. These two binary vector constructs have a selectable marker that differs from the GUS construct, so that they can be used to superinfect with a second T-DNA construct. When each of these constructs is transformed into the T2 plants described above, the level of GUS expression is determined for the doubly-transformed T1 progeny. Those T1 plants overexpressing the candidate protein are expected to have lower levels of GUS expression due to increased silencing. If a difference is not detected in those T1 plants, lines homozygous for the candidate overexpression construct can be produced in the T2 generation and examined.

Alternatively, a nucleotide sequence set forth in any one of SEQ ID NOs provided in table 1 above is included in a construct as described above and is used for overexpression of the respective candidate polypeptide.

### Example 10

### Promoter Analysis

The gene chip experiment described above in Example 1 are designed to uncover genes that are expressed during PTGS. Candidate promoters are identified based upon the expression profiles of the associated transcripts representatives of which are provided in SEQ ID NOs: 1-341

Candidate promoters are obtained by PCR and fused to a GUS reporter gene containing an intron. Both histochemical and fluormetric GUS assays are carried out on stably transformed rice and maize plants and GUS activity is detected in the transformants.

Further, transient assays with the promoter::GUS constructs are carried out in rice embryogenic callus and GUS activity is detected by histochemical staining according the protocol described below.

10.1 Construction of binary Promoter::Reporter Plasmids

To construct a binary promoter:: reporter plasmid for rice transformation a vector containing a promoter of interest (i.e., the DNA sequence 5′ of the initiation codon for the gene of interest) is used, which results from recombination in a BP reaction between a PCR product using the promoter of interest as a template and pDONR201™, producing an entry vector. The regulatory/promoter sequence is fused to the GUS reporter gene (Jefferson et al, 1987) by recombination using GATEWAY™ Technology according to manufacturers protocol as described in the Instruction Manual (GATEWAY™ Cloning Technology, GIBCO BRL, Rockville, Md. http://www.lifetech.com/).

Briefly, the Gateway Gus-intron-Gus (GIG)/NOS expression cassette is ligated into pNOV2117 binary vector in 5′ to 3′ orientation. The 4.1 kB expression cassette is ligated into the Kpn-I site of pNOV2117, then clones are screened for orientation to obtain pNOV2346, a GATEWAY™ adapted binary destination vector.

The promoter fragment in the entry vector is recombined via the LR reaction with the binary destination vector containing the GUS coding region with an intron that has an attR site 5′ to the GUS reporter, producing a binary vector with a promoter fused to the GUS reporter (pNOVCANDProm). The orientation of the inserted fragment is maintained by the att sequences and the final construct is verified by sequencing. The construct is then transformed into Agrobacterium tumefaciens strains by electroporation as described herein previously.

### Example 10.2

### Transient Expression Analysis of Candidate Promoters in Rice Embryogenic Callus

Materials

Embryogenic rice callus (Kaybonett cultivar)

LBA 4404 Agrobacterium strains

KCMS liquid media for re-suspending bacterial pellet

200 mM stock (40 mg/ml) Acetosyringone

Sterile filter paper discs (8.5 mm in diameter)

LB spec liquid culture

MS-CIM media plates

MS-AS plates (co-cultivation plates)

MS-Tim plates (recovery plates)

Gus staining solution

Methods

Induction of Embryogenic Callus

1. Sterilize mature Kaybonett rice seeds in 40% ultra Clorox, 1 drop Tween 20, for 40 min.

2. Rinse with sterile water and plate on MS-CIM media (12 seeds/plate)

3. Grow in dark for four weeks.

4. Isolate embryogenic calli from scutellum to MS-CIM

5. Let grow in dark 8 days before use for transformation

Agrobacterium Preparation and Induction

1. Start 6 mL shaking cultures of LBA4404 Agrobacterium strains harboring rice promoter binary plasmids.

2. Grow the cultures at room temperature for 48 hrs in the rotary shaker.

3. Spin down the cultures at 8,000 rpm at 4° C. and re-suspend bacterial pellets in 10 ml of KCMS media supplemented with 100 μM Acetosyringone.

4. Place in the shaker at room temp for 1 hr for induction of Agrobacterium virulence genes.

5. In a sterile hood dilute Agrobacterium cultures 1:3 in KSMS media and transfer diluted cultures into deep petri dishes.

Inoculation of Plant Material and Staining

6. In a sterile hood transfer embryogenic callus into diluted Agrobacerium solution and incubate for 30 minutes.

7. In a sterile hood blot callus tissue on sterile filter paper and transfer on MS-AS plates.

8. Co-culture plates in 22° C. growth chamber in the dark for two days.

9. In a sterile hood transfer callus tissue to MS-Tim plates for the tissue recovery (the presence of Timentin will prevent Agrobacterium growth).

10. Incubate tissue on MS-Tim media for two days at 22° C. in the dark.

11. Remove callus tissue from the plates and stain for 48 hrs. in GUS staining solution.

12. De-stain tissue in 70% EtOH for 24 hours.

Recipies

KCMS media (liquid), pH to 5.5

100 ml/l MS Major Salts, 10 ml/l MS Minor Salts, 5 ml/l MS iron stock, 0.5M K₂HPO₄, 0.1 mg/ml Myo-Inositol,

1.3 μg/ml Thiamine, 0.2 g/ml 2,4-D (1 mg/ml), 0.1 g/ml Kinetin, 3% Sucrose, 100 μM Acetosyringone

MS-CIM media, pH 5.8

MS Basal salt (4.3 g/L), B5 Vitamins (200×) (5 m/L), 2% Sucrose (20 g/L), Proline (500 mg/L), Glutamine (500 mg/L), Casein Hydrolysate (300 mg/L), 2 μg/ml 2,4-D, Phytagel (3 g/L)

MS-As Medium, pH 5.8

MS Basal salt (4.3 g/L), B5 Vitamins (200×) (5 m/L), 2% Sucrose (20 g/L), Proline (500 mg/L), Glutamine (500 mg/L), Casein Hydrolysate (300 mg/L), 2μg/ml 2,4-D, Phytagel (3 g/L), 200 μM Acetosyringone

MS-Tim media, pH 5.8

MS Basal salt (4.3 g/L), B5 Vitamins (200×) (5 m/L), 2% Sucrose (20 g/L), Proline (500 mg/L), Glutamine (500 mg/L), Casein Hydrolysate (300 mg/L), 2 μg/ml 2,4-D, Phytagel (3 g/L), 400 mg/l Timentin

Gus staining solution, pH 7

0.3M Mannitol; 0.02M EDTA, pH=7.0; 0.04 NaH₂PO₄; 1 mM x-gluc

The binary Promoter: :Reporter Plasmids described in Example 10.1 above can also be used for stable transformation of rice and maize plants according to the protocols provided in Examples 8.1 and 8.2, respectively.

### Example 11

### Analysis of Mutant and Transgenic Plant Material

Two tiers of assays are can be used for analysis of the mutant and transgenic plant material.

Near InfraRed (NIR) Spectrophometric Analysis of Seeds

NIR enables evaluation of changes in starch, oil, protein and fiber content at very high throughput (1 sample/sec).

DIA or MRI Imaging

DIA or MRI imaging allows observation of gross morphology and surface area of major seed tissues and compartments (embryo, aleurone, endosperm, seed coat). Transgenic lines can also be physically sectioned and directly observed for changes in seed compartment morphology.

Lines showing alterations in grain composition will be advanced to a second tier of assays dependent upon the nature of the change detected:

1) Protein track: 1-D and 2-D protein gels Protein profiles

HPLC Amino acid profiles

DNTB or papain staining Protein redox status

GC N/C/S ratios

2) Starch track: Iodine staining Content, branching

Glucose-6-P analysis Phosphorylation level

3) Oils track: GC Oil, fatty acid profile

### Example 12

### Chromosomal Markers to Identify the Location of a Nucleic Acid Sequence

The sequences of the present invention can also be used for SSR mapping. SSR mapping in rice has been described by Miyao et al. (DNA Res 3:233 (1996)) and Yang et al. (Mol Gen Genet 245:187 (1994)), and in maize by Ahn et al. (Mol Gen Genet 241:483 (1993)). SSR mapping can be achieved using various methods. In one instance, polymorphisms are identified when sequence specific probes flanking an SSR contained within a sequence are made and used in polymerase chain reaction (PCR) assays with template DNA from two or more individuals or, in plants, near isogenic lines. A change in the number of tandem repeats between the SSR-flanking sequence produces differently sized fragments (U.S. Pat. No. 5,766,847). Alternatively, polymorphisms can be identified by using the PCR fragment produced from the SSR-flanking sequence specific primer reaction as a probe against Southern blots representing different individuals (Refseth et al., Electrophoresis 18:1519 (1997)). Rice SSRs can be used to map a molecular marker closely linked to functional gene, as described by Akagi et al. (Genome 39:205 (1996)).

The sequences of the present invention can be used to identify and develop a variety of microsatellite markers, including the SSRs described above, as genetic markers for comparative analysis and mapping of genomes.

Many of the polynucleotides listed in Tables 2 to 11 contain at least 3 consecutive di-, tri- or tetranucleotide repeat units in their coding region that can potentially be developed into SSR markers. Trinucleotide motifs that can be commonly found in the coding regions of said polynucleotides and easily identified by screening the polynucleotides sequences for said motifs are, for example: CGG; GCC, CGC, GGC, etc. Once such a repeat unit has been found, primers can be designed which are complementary to the region flanking the repeat unit and used in any of the methods described below.

Sequences of the present invention can also be used in a variation of the SSR technique known as inter-SSR (ISSR), which uses microsatellite oligonucleotides as primers to amplify genomic segments different from the repeat region itself (Zietkiewicz et al., Genomics 20:176 (1994)). ISSR employs oligonucleotides based on a simple sequence repeat anchored or not at their 5′- or 3′-end by two to four arbitrarily chosen nucleotides, which triggers site-specific annealing and initiates PCR amplification of genomic segments which are flanked by inversely orientated and closely spaced repeat sequences. In one embodiment of the present invention, microsatellite markers as disclosed herein, or substantially similar sequences or allelic variants thereof, may be used to detect the appearance or disappearance of markers indicating genomic instability as described by Leroy et al. (Electron. J Biotechnol, 3(2), at http://www.ejb.org (2000)), where alteration of a fingerprinting pattern indicated loss of a marker corresponding to a part of a gene involved in the regulation of cell proliferation. Microsatellite markers are useful for detecting genomic alterations such as the change observed by Leroy et al. (Electron. J Biotechnol, 3(2), supra (2000)) which appeared to be the consequence of microsatellite instability at the primer binding site or modification of the region between the microsatellites, and illustrated somaclonal variation leading to genomic instability. Consequently, sequences of the present invention are useful for detecting genomic alterations involved in somaclonal variation, which is an important source of new phenotypes.

In addition, because the genomes of closely related species are largely syntenic (that is, they display the same ordering of genes within the genome), these maps can be used to isolate novel alleles from wild relatives of crop species by positional cloning strategies. This shared synteny is very powerful for using genetic maps from one species to map genes in another. For example, a gene mapped in rice provides information for the gene location in maize and wheat.

### Example 13

### Quantitative Trait Linked Breeding

Various types of maps can be used with the sequences of the invention to identify Quantitative Trait Loci (QTLs) for a variety of uses, including marker-assisted breeding.

Many important crop traits are quantitative traits and result from the combined interactions of several genes. These genes reside at different loci in the genome, often on different chromosomes, and generally exhibit multiple alleles at each locus. Developing markers, tools, and methods to identify and isolate the QTLs involved in a trait, enables marker-assisted breeding to enhance desirable traits or suppress undesirable traits. The sequences disclosed herein can be used as markers for QTLs to assist marker-assisted breeding. The sequences of the invention can be used to identify QTLs and isolate alleles as described by Li et al. in a study of QTLs involved in resistance to a pathogen of rice. (Li et al., Mol Gen Genet 261:58 (1999)). In addition to isolating QTL alleles in rice, other cereals, and other monocot and dicot crop species, the sequences of the invention can also be used to isolate alleles from the corresponding QTL(s) of wild relatives. Transgenic plants having various combinations of QTL alleles can then be created and the effects of the combinations measured. Once an ideal allele combination has been identified, crop improvement can be accomplished either through biotechnological means or by directed conventional breeding programs. (Flowers et al., J Exp Bot 51:99 (2000); Tanksley and McCouch, Science 277:1063 (1997)).

### Example 14

### Marker-Assisted Breeding

Markers or genes associated with specific desirable or undesirable traits are known and used in marker assisted breeding programs. It is particularly beneficial to be able to screen large numbers of markers and large numbers of candidate parental plants or progeny plants. The methods of the invention allow high volume, multiplex screening for numerous markers from numerous individuals simultaneously.

Markers or genes associated with specific desirable or undesirable traits are known and used in marker assisted breeding programs. It is particularly beneficial to be able to screen large numbers of markers and large numbers of candidate parental plants or progeny plants. The methods of the invention allow high volume, multiplex screening for numerous markers from numerous individuals simultaneously.

A multiplex assay is designed providing SSRs specific to each of the markers of interest. The SSRs are linked to different classes of beads. All of the relevant markers may be expressed genes, so RNA or cDNA techniques are appropriate. RNA is extracted from root tissue of 1000 different individual plants and hybridized in parallel reactions with the different classes of beads. Each class of beads is analyzed for each sample using a microfluidics analyzer. For the classes of beads corresponding to qualitative traits, qualitative measures of presence or absence of the target gene are recorded. For the classes of beads corresponding to quantitative traits, quantitative measures of gene activity are recorded. Individuals showing activity of all of the qualitative genes and highest expression levels of the quantitative traits are selected for further breeding steps. In procedures wherein no individuals have desirable results for all the measured genes, individuals having the most desirable, and fewest undesirable, results are selected for further breeding steps. In either case, progeny are screened to further select for homozygotes with high quantitative levels of expression of the quantitative traits.

### Example 15

### Method of Modifying the Gene Frequency

The invention further provides a method of modifying the frequency of a gene in a plant population, including the steps of: identifying an SSR within a coding region of a gene; screening a plurality of plants using the SSR as a marker to determine the presence or absence of the gene in an individual plant; selecting at least one individual plant for breeding based on the presence or absence of the gene; and breeding at least one plant thus selected to produce a population of plants having a modified frequency of the gene. The identification of the SSR within the coding region of a gene can be accomplished based on sequence similarity between the nucleic acid molecules of the invention and the region within the gene of interest flanking the SSR.

TABLE 3
This table illustrates the start and end points and the
sequence of tri- and tetra-nucleotide repeat units in
the coding region of a selection of SEQ ID NOs.

| SeqID | Start | End | Sequence |
|---|---|---|---|
| 9 | 429 | 443 | CCG |
| 13 | 580 | 594 | CCG |
| 19 | 548 | 562 | CGC |
| | 1031 | 1048 | GCG |
| 21 | 155 | 172 | ATG |
| | 507 | 521 | CGC |
| | 579 | 593 | GTC |
| | 752 | 766 | CCG |
| 39 | 762 | 776 | ACC |
| 49 | 280 | 294 | CGG |
| 61 | 63 | 80 | AGG |
| 63 | 579 | 593 | CGG |
| 69 | 65 | 79 | CGG |
| 71 | 755 | 769 | CCG |
| 93 | 27 | 41 | CGC |
| 115 | 437 | 451 | GCG |
| 129 | 124 | 141 | AGC |
| 131 | 89 | 103 | CGC |
| 145 | 752 | 769 | CCG |
| 159 | 487 | 501 | CCG |
| 161 | 612 | 626 | CAG |
| 175 | 163 | 183 | GGT |
| 181 | 58 | 72 | CTC |
| | 852 | 866 | CGG |
| 199 | 482 | 496 | CGC |
| | 964 | 981 | GCG |
| 205 | 284 | 298 | AGC |
| 211 | 165 | 179 | CGG |
| 215 | 46 | 60 | AAC |
| | 68 | 82 | AGC |
| | 134 | 151 | GCG |
| | 550 | 567 | ACG |
| 217 | 17 | 37 | ACC |
| 225 | 366 | 380 | CGC |
| 229 | 65 | 82 | CCG |
| 231 | 85 | 99 | CCG |
| 237 | 73 | 87 | ACC |
| | 282 | 302 | CGG |
| 239 | 61 | 78 | CTG |
| 259 | 347 | 367 | GCG |
| 269 | 1032 | 1046 | CGG |
| 279 | 899 | 919 | CGG |
| 297 | 706 | 720 | GGC |
| 303 | 633 | 647 | CAC |
| 305 | 1086 | 1100 | CGG |
| 309 | 523 | 537 | GGT |
| 313 | 36 | 53 | AGG |
| 317 | 527 | 553 | CCG |
| 327 | 1085 | 1108 | GCG |
| 329 | 3226 | 3240 | GCA |

TABLE 4
Swiss-Prot Data

Seq ID: 1
Accession: Q9FZ27
Swissprot_id: GL22_ARATH
Gi_number: 18202917
Description: Germin-like protein subfamily 2
member 2 precursor
Seq ID: 3
Accession: P27164
Swissprot_id: CAL3_PETHY
Gi_number: 115492
Description: CALMODULIN-RELATED PROTEIN
Seq ID: 7
Accession: P56578
Swissprot_id: MLF3_MALFU
Gi_number: 3914387
Description: Allergen Mal f 3 (MF2)
Seq ID: 9
Accession: Q41001
Swissprot_id: BCP_PEA
Gi_number: 2493318
Description: Blue copper protein precursor
Seq ID: 11
Accession: P42736
Swissprot_id: CDI3_ARATH
Gi_number: 1168862
Description: CADMIUM-INDUCED PROTEIN AS30
Seq ID: 13
Accession: P04839
Swissprot_id: C24B_HUMAN
Gi_number: 115211
Description: Cytochrome B-245 heavy chain (P22
phagocyte B-cytochrome) (Neutrophil cytochrome B,
91 kDa polypeptide) (CGD91-PHOX) (GP91-PHOX)
(Heme binding membrane glycoprotein GP91PHOX)
(Cytochrome B(558) beta chain) (Superoxide-
generating NADPH oxidase heavy cha>
Seq ID: 15
Accession: P37835
Swissprot_id: PER2_ORYSA
Gi_number: 585662
Description: Peroxidase precursor
Seq ID: 17
Accession: P80679
Swissprot_id: PERX_ARMRU
Gi_number: 1730490
Description: Peroxidase
Seq ID: 19
Accession: P13983
Swissprot_id: EXTN_TOBAC
Gi_number: 119714
Description: Extensin precursor (Cell wall
hydroxyproline-rich glycoprotein)
Seq ID: 21
Accession: P42736
Swissprot_id: CDI3_ARATH
Gi_number: 1168862
Description: CADMIUM-INDUCED PROTEIN AS30
Seq ID: 23
Accession: Q05968
Swissprot_id: PR1_HORVU
Gi_number: 548592
Description: PATHOGENESIS-RELATED PROTEIN
1 PRECURSOR
Seq ID: 25
Accession: P14009
Swissprot_id: 14KD_DAUCA
Gi_number: 112697
Description: 14 KD PROLINE-RICH PROTEIN
DC2.15 PRECURSOR
Seq ID: 27
Accession: P31691
Swissprot_id: ADT_ORYSA
Gi_number: 399015
Description: ADP,ATP carrier protein,
mitochondrial precursor (ADP/ATP
translocase) (Adenine nucleotide
translocator) (ANT)
Seq ID: 29
Accession: P28968
Swissprot_id: VGLX_HSVEB
Gi_number: 138350
Description: GLYCOPROTEIN X PRECURSOR
Seq ID: 31
Accession: Q43257
Swissprot_id: C7C4_MAIZE
Gi_number: 5921189
Description: CYTOCHROME P450 71C4
Seq ID: 33
Accession: P27061
Swissprot_id: PPA1_LYCES
Gi_number: 130718
Description: Acid phosphatase precursor 1
Seq ID: 35
Accession: P41153
Swissprot_id: HSF8_LYCPE
Gi_number: 729775
Description: HEAT SHOCK FACTOR PROTEIN
HSF8 (HEAT SHOCK TRANSCRIPTION FACTOR 8)
(HSTF 8) (HEAT STRESS TRANSCRIPTION FACTOR)
Seq ID: 37
Accession: Q02438
Swissprot_id: E13E_HORVU
Gi_number: 1352328
Description: GLUCAN ENDO-1,3-BETA-
GLUCOSIDASE GV ((1->3)-BETA-GLUCAN
ENDOHYDROLASE GV) ((1->3)-BETA-GLUCANASE
ISOENZYME GV) (BETA-1,3-ENDOGLUCANASE GV)
Seq ID: 39
Accession: P43293
Swissprot_id: NAK_ARATH
Gi_number: 1171642
Description: Probable serine/threonine-
protein kinase NAK
Seq ID: 41
Accession: P33679
Swissprot_id: ZEAM_MAIZE
Gi_number: 1731426
Description: Zeamatin precursor
Seq ID: 43
Accession: P43216
Swissprot_id: MPH1_HOLLA
Gi_number: 1171005
Description: MAJOR POLLEN ALLERGEN HOL
L 1 PRECURSOR (HOL L I) (HOL L 1.0101
AND 1.0102)
Seq ID: 45
Accession: Q42429
Swissprot_id: AGL8_SOLTU
Gi_number: 3913001
Description: Agamous-like MADS box protein
AGL8 homolog (POTM1-1)
Seq ID: 47
Accession: P21997
Swissprot_id: SSGP_VOLCA
Gi_number: 134920
Description: SULFATED SURFACE GLYCOPROTEIN
185 (SSG 185)
Seq ID: 49
Accession: Q00451
Swissprot_id: PRF1_LYCES
Gi_number: 1709767
Description: 36.4 KD PROLINE-RICH PROTEIN
Seq ID: 51
Accession: P23586
Swissprot_id: STP1_ARATH
Gi_number: 134976
Description: GLUCOSE TRANSPORTER (SUGAR CARRIER)
Seq ID: 53
Accession: Q40412
Swissprot_id: ABA2_NICPL
Gi_number: 5902707
Description: Zeaxanthin epoxidase, chloroplast
precursor
Seq ID: 55
Accession: P51074
Swissprot_id: ANX4_FRAAN
Gi_number: 1703318
Description: Annexin-like protein RJ4
Seq ID: 57
Accession: P47179
Swissprot_id: DAN4_YEAST
Gi_number: 1352944
Description: Cell wall protein DAN4 precursor
Seq ID: 59
Accession: P80679
Swissprot_id: PERX_ARMRU
Gi_number: 1730490
Description: Peroxidase
Seq ID: 61
Accession: P37835
Swissprot_id: PER2_ORYSA
Gi_number: 585662
Description: Peroxidase precursor
Seq ID: 63
Accession: P08640
Swissprot_id: AMYH_YEAST
Gi_number: 728850
Description: GLUCOAMYLASE S1/S2 PRECURSOR
(GLUCAN 1,4-ALPHA-GLUCOSIDASE) (1,4-ALPHA-
D-GLUCAN GLUCOHYDROLASE)
Seq ID: 65
Accession: Q05049
Swissprot_id: MUC1_XENLA
Gi_number: 585527
Description: INTEGUMENTARY MUCIN C.1 (FIM-C.1)
Seq ID: 67
Accession: P24626
Swissprot_id: CHI1_ORYSA
Gi_number: 116303
Description: BASIC ENDOCHITINASE 1 PRECURSOR
Seq ID: 69
Accession: P35694
Swissprot_id: BRU1_SOYBN
Gi_number: 543905
Description: BRASSINOSTEROID-REGULATED PROTEIN
BRU1
Seq ID: 71
Accession: P29022
Swissprot_id: CHIA_MAIZE
Gi_number: 116329
Description: ENDOCHITINASE A PRECURSOR (SEED
CHITINASE A)
Seq ID: 73
Accession: P52914
Swissprot_id: NTPA_PEA
Gi_number: 1709358
Description: Nucleoside-triphosphatase
(Nucleoside triphosphate phosphohydrolase)
(NTPase) (Apyrase)
Seq ID: 75
Accession: Q9SW70
Swissprot_id: SRP_VITRI
Gi_number: 15214303
Description: Stress-related protein
Seq ID: 77
Accession: P28968
Swissprot_id: VGLX_HSVEB
Gi_number: 138350
Description: GLYCOPROTEIN X PRECURSOR
Seq ID: 79
Accession: P27644
Swissprot_id: PGLR_AGRTU
Gi_number: 129937
Description: POLYGALACTURONASE (PECTINASE) (PGL)
Seq ID: 81
Accession: P40387
Swissprot_id: TPS1_SCHPO
Gi_number: 730984
Description: ALPHA,ALPHA-TREHALOSE-PHOSPHATE
SYNTHASE [UDP-FORMING] (TREHALOSE-6-PHOSPHATE
SYNTHASE) (UDP-GLUCOSE-GLUCOSEPHOSPHATE
GLUCOSYLTRANSFERASE)
Seq ID: 83
Accession: P33126
Swissprot_id: HS82_ORYSA
Gi_number: 417154
Description: HEAT SHOCK PROTEIN 82
Seq ID: 85
Accession: P35792
Swissprot_id: PR12_HORVU
Gi_number: 548588
Description: PATHOGENESIS-RELATED PROTEIN
PRB1-2 PRECURSOR
Seq ID: 87
Accession: O04985
Swissprot_id: HBL2_ORYSA
Gi_number: 17432965
Description: Non-symbiotic hemoglobin 2 (Hb2)
Seq ID: 89
Accession: P24632
Swissprot_id: HS22_MAIZE
Gi_number: 123553
Description: 17.8 KD CLASS II HEAT SHOCK PROTEIN
Seq ID: 91
Accession: P23444
Swissprot_id: H1_MAIZE
Gi_number: 121950
Description: HISTONE H1
Seq ID: 93
Accession: P51103
Swissprot_id: DFRA_CALCH
Gi_number: 1706369
Description: DIHYDROFLAVONOL-4-REDUCTASE (DFR)
(DIHYDROKAEMPFEROL 4-REDUCTASE)
Seq ID: 95
Accession: P51847
Swissprot_id: DCP1_ORYSA
Gi_number: 1706325
Description: PYRUVATE DECARBOXYLASE ISOZYME 1
(PDC)
Seq ID: 97
Accession: P22953
Swissprot_id: HS71_ARATH
Gi_number: 12643273
Description: Heat shock cognate 70 kDa protein
1 (Hsc70.1)
Seq ID: 99
Accession: Q9WTV7
Swissprot_id: RNFB_MOUSE
Gi_number: 13124535
Description: RING FINGER PROTEIN 12 (LIM
DOMAIN INTERACTING RING FINGER PROTEIN) (RING
FINGER LIM DOMAIN-BINDING PROTEIN) (R-LIM)
Seq ID: 101
Accession: P93147
Swissprot_id: C81E_GLYEC
Gi_number: 5915842
Description: CYTOCHROME P450 81E1 (ISOFLAVONE
2′-HYDROXYLASE) (P450 91A4) (CYP GE-3)
Seq ID: 103
Accession: P24805
Swissprot_id: TSJT_TOBAC
Gi_number: 136452
Description: STEM-SPECIFIC PROTEIN TSJT1
Seq ID: 105
Accession: P12257
Swissprot_id: GUB2_HORVU
Gi_number: 121773
Description: LICHENASE II PRECURSOR (ENDO-
BETA-1,3-1,4 GLUCANASE II) ((1->3,1->4)-BETA-
GLUCANASE ISOENZYME EII)
Seq ID: 107
Accession: P25776
Swissprot_id: ORYA_ORYSA
Gi_number: 129231
Description: ORYZAIN ALPHA CHAIN PRECURSOR
Seq ID: 109
Accession: P33679
Swissprot_id: ZEAM_MAIZE
Gi_number: 1731426
Description: Zeamatin precursor
Seq ID: 111
Accession: P12653
Swissprot_id: GTH1_MAIZE
Gi_number: 121695
Description: GLUTATHIONE S-TRANSFERASE I
(GST-I) (GST-29) (GST CLASS-PHI)
Seq ID: 113
Accession: P52839
Swissprot_id: FSTL_ARATH
Gi_number: 1706917
Description: Flavonol sulfotransferase-like
(RaRO47)
Seq ID: 115
Accession: P38564
Swissprot_id: MNBA_MAIZE
Gi_number: 1346559
Description: DNA-BINDING PROTEIN MNB1A
Seq ID: 117
Accession: P11675
Swissprot_id: IE18_PRVIF
Gi_number: 124178
Description: IMMEDIATE-EARLY
PROTEIN IE180
Seq ID: 119
Accession: P31541
Swissprot_id: CLAA_LYCES
Gi_number: 399212
Description: ATP-dependent clp protease
ATP-binding subunit clpA homolog CD4A,
chloroplast precursor
Seq ID: 121
Accession: P52157
Swissprot_id: RHO_STRLI
Gi_number: 1710269
Description: TRANSCRIPTION TERMINATION
FACTOR RHO
Seq ID: 123
Accession: P13730
Swissprot_id: SGS3_DROER
Gi_number: 134466
Description: SALIVARY GLUE PROTEIN SGS-3
PRECURSOR
Seq ID: 125
Accession: O48670
Swissprot_id: RERA_ARATH
Gi_number: 6225938
Description: RER1A protein (AtRER1A)
Seq ID: 127
Accession: P50165
Swissprot_id: TRNH_DATST
Gi_number: 1717755
Description: TROPINONE REDUCTASE HOMOLOG (P29X)
Seq ID: 129
Accession: P12978
Swissprot_id: EBN2_EBV
Gi_number: 119111
Description: EBNA-2 NUCLEAR PROTEIN
Seq ID: 131
Accession: P43293
Swissprot_id: NAK_ARATH
Gi_number: 1171642
Description: Probable serine/threonine-protein
kinase NAK
Seq ID: 133
Accession: P52594
Swissprot_id: NUPL_HUMAN
Gi_number: 1709416
Description: NUCLEOPORIN-LIKE PROTEIN RIP
(HIV-1 REV-BINDING PROTEIN) (REV INTERACTING
PROTEIN) (REV/REX ACTIVATION DOMAIN-BINDING
PROTEIN)
Seq ID: 135
Accession: Q41819
Swissprot_id: IAAG_MAIZE
Gi_number: 2501499
Description: INDOLE-3-ACETATE BETA-
GLUCOSYLTRANSFERASE (IAA-GLU SYNTHETASE)
((URIDINE 5′-DIPHOSPHATE-GLUCOSE:INDOL-
3-YLACETYL)-BETA-D-GLUCOSYL TRANSFERASE)
Seq ID: 137
Accession: P14009
Swissprot_id: 14KD_DAUCA
Gi_number: 112697
Description: 14 KD PROLINE-RICH PROTEIN
DC2.15 PRECURSOR
Seq ID: 139
Accession: P42736
Swissprot_id: CDI3_ARATH
Gi_number: 1168862
Description: CADMIUM-INDUCED PROTEIN AS30
Seq ID: 143
Accession: Q99962
Swissprot_id: SH32_HUMAN
Gi_number: 10720276
Description: SH3-containing GRB2-like protein
2 (SH3 domain protein 2A) (Endophilin 1)
(EEN-B1)
Seq ID: 145
Accession: Q9FG65
Swissprot_id: C911_ARATH
Gi_number: 13878373
Description: CYTOCHROME P450 91A1
Seq ID: 147
Accession: P93329
Swissprot_id: NO20_MEDTR
Gi_number: 3914142
Description: EARLY NODULIN 20 PRECURSOR (N-20)
Seq ID: 149
Accession: P51103
Swissprot_id: DFRA_CALCH
Gi_number: 1706369
Description: DIHYDROFLAVONOL-4-REDUCTASE (DFR)
(DIHYDROKAEMPFEROL 4-REDUCTASE)
Seq ID: 157
Accession: P52594
Swissprot_id: NUPL_HUMAN
Gi_number: 1709416
Description: NUCLEOPORIN-LIKE PROTEIN RIP
(HIV-1 REV-BINDING PROTEIN) (REV INTERACTING
PROTEIN) (REV/REX ACTIVATION DOMAIN-BINDING
PROTEIN)
Seq ID: 159
Accession: Q00451
Swissprot_id: PRF1_LYCES
Gi_number: 1709767
Description: 36.4 KD PROLINE-RICH PROTEIN
Seq ID: 161
Accession: Q38836
Swissprot_id: AG11_ARATH
Gi_number: 12229648
Description: Agamous-like MADS box protein AGL11
Seq ID: 165
Accession: O04985
Swissprot_id: HBL2_ORYSA
Gi_number: 17432965
Description: Non-symbiotic hemoglobin 2 (Hb2)
Seq ID: 167
Accession: P80679
Swissprot_id: PERX_ARMRU
Gi_number: 1730490
Description: Peroxidase
Seq ID: 169
Accession: Q9Y2L1
Swissprot_id: RR44_HUMAN
Gi_number: 7674415
Description: EXOSOME COMPLEX EXONUCLEASE
RRP44 (RIBOSOMAL RNA PROCESSING PROTEIN 44)
(DIS3 PROTEIN HOMOLOG)
Seq ID: 171
Accession: P27061
Swissprot_id: PPA1_LYCES
Gi_number: 130718
Description: Acid phosphatase precursor 1
Seq ID: 173
Accession: P35792
Swissprot_id: PR12_HORVU
Gi_number: 548588
Description: PATHOGENESIS-RELATED PROTEIN
PRB1-2 PRECURSOR
Seq ID: 175
Accession: O04682
Swissprot_id: PTI6_LYCES
Gi_number: 7531181
Description: PATHOGENESIS-RELATED GENES
TRANSCRIPTIONAL ACTIVATOR PTI6
Seq ID: 177
Accession: Q05968
Swissprot_id: PR1_HORVU
Gi_number: 548592
Description: PATHOGENESIS-RELATED PROTEIN
1 PRECURSOR
Seq ID: 179
Accession: P43254
Swissprot_id: COP1_ARATH
Gi_number: 1169012
Description: COP1 regulatory protein (FUSCA
protein FUS1)
Seq ID: 181
Accession: O48958
Swissprot_id: C7E1_SORBI
Gi_number: 5915841
Description: Cytochrome P450 71E1
Seq ID: 183
Accession: P14080
Swissprot_id: PAP2_CARPA
Gi_number: 2507252
Description: CHYMOPAPAIN PRECURSOR (PAPAYA
PROTEINASE II) (PPII)
Seq ID: 185
Accession: P24102
Swissprot_id: PERE_ARATH
Gi_number: 129817
Description: Basic peroxidase E precursor
Seq ID: 187
Accession: P11675
Swissprot_id: IE18_PRVIF
Gi_number: 124178
Description: IMMEDIATE-EARLY PROTEIN IE180
Seq ID: 189
Accession: Q96324
Swissprot_id: GTH7_ARATH
Gi_number: 12230147
Description: Glutathione S-transferase (GST
class phi)
Seq ID: 191
Accession: P31691
Swissprot_id: ADT_ORYSA
Gi_number: 399015
Description: ADP,ATP carrier protein,
mitochondrial precursor (ADP/ATP
translocase) (Adenine nucleotide
translocator) (ANT)
Seq ID: 193
Accession: P50165
Swissprot_id: TRNH_DATST
Gi_number: 1717755
Description: TROPINONE REDUCTASE HOMOLOG (P29X)
Seq ID: 197
Accession: P41152
Swissprot_id: HSF3_LYCPE
Gi_number: 729774
Description: HEAT SHOCK FACTOR PROTEIN HSF30
(HEAT SHOCK TRANSCRIPTION FACTOR 30) (HSTF
30) (HEAT STRESS TRANSCRIPTION FACTOR)
Seq ID: 199
Accession: P13730
Swissprot_id: SGS3_DROER
Gi_number: 134466
Description: SALIVARY GLUE PROTEIN SGS-3
PRECURSOR
Seq ID: 201
Accession: Q41819
Swissprot_id: IAAG_MAIZE
Gi_number: 2501499
Description: INDOLE-3-ACETATE BETA-
GLUCOSYLTRANSFERASE (IAA-GLU SYNTHETASE) ((URIDINE
5′-DIPHOSPHATE-GLUCOSE:INDOL-3-YLACETYL)-
BETA-D-GLUCOSYL TRANSFERASE)
Seq ID: 203
Accession: P35793
Swissprot_id: PR13_HORVU
Gi_number: 548589
Description: PATHOGENESIS-RELATED PROTEIN PRB1-3
PRECURSOR (PR-1B) (HV-8)
Seq ID: 205
Accession: P56578
Swissprot_id: MLF3_MALFU
Gi_number: 3914387
Description: Allergen Mal f 3 (MF2)
Seq ID: 207
Accession: P35694
Swissprot_id: BRU1_SOYBN
Gi_number: 543905
Description: BRASSINOSTEROID-REGULATED
PROTEIN BRU1
Seq ID: 209
Accession: Q43793
Swissprot_id: G6PC_TOBAC
Gi_number: 3023817
Description: GLUCOSE-6-PHOSPHATE 1-
DEHYDROGENASE, CHLOROPLAST PRECURSOR (G6PD)
Seq ID: 211
Accession: P29022
Swissprot_id: CHIA_MAIZE
Gi_number: 116329
Description: ENDOCHITINASE A PRECURSOR
(SEED CHITINASE A)
Seq ID: 213
Accession: P27322
Swissprot_id: HS72_LYCES
Gi_number: 123620
Description: HEAT SHOCK COGNATE 70 KD PROTEIN 2
Seq ID: 215
Accession: P38564
Swissprot_id: MNBA_MAIZE
Gi_number: 1346559
Description: DNA-BINDING PROTEIN MNB1A
Seq ID: 217
Accession: P11675
Swissprot_id: IE18_PRVIF
Gi_number: 124178
Description: IMMEDIATE-EARLY PROTEIN IE180
Seq ID: 219
Accession: Q9SW70
Swissprot_id: SRP_VITRI
Gi_number: 15214303
Description: Stress-related protein
Seq ID: 221
Accession: P15737
Swissprot_id: E13B_HORVU
Gi_number: 119003
Description: Glucan endo-1,3-beta-glucosidase
GII precursor ((1->3)-beta-glucan endohydrolase
GII) ((1->3)-beta-glucanase isoenzyme GII)
(Beta-1,3-endoglucanase GII)
Seq ID: 223
Accession: P51846
Swissprot_id: DCP2_TOBAC
Gi_number: 1706330
Description: PYRUVATE DECARBOXYLASE ISOZYME 2
(PDC)
Seq ID: 225
Accession: P11955
Swissprot_id: CHI1_HORVU
Gi_number: 2506281
Description: 26 KD ENDOCHITINASE 1 PRECURSOR
Seq ID: 227
Accession: P21997
Swissprot_id: SSGP_VOLCA
Gi_number: 134920
Description: SULFATED SURFACE GLYCOPROTEIN 185
(SSG 185)
Seq ID: 229
Accession: P33679
Swissprot_id: ZEAM_MAIZE
Gi_number: 1731426
Description: Zeamatin precursor
Seq ID: 231
Accession: P27061
Swissprot_id: PPA1_LYCES
Gi_number: 130718
Description: Acid phosphatase precursor 1
Seq ID: 233
Accession: P54781
Swissprot_id: ERG5_YEAST
Gi_number: 1706693
Description: CYTOCHROME P450 61 (C-22 STEROL
DESATURASE)
Seq ID: 235
Accession: Q9SFF9
Swissprot_id: GL17_ARATH
Gi_number: 18203443
Description: Germin-like protein subfamily 1
member 7 precursor
Seq ID: 237
Accession: P11955
Swissprot_id: CHI1_HORVU
Gi_number: 2506281
Description: 26 KD ENDOCHITINASE 1 PRECURSOR
Seq ID: 239
Accession: O04886
Swissprot_id: PME1_CITSI
Gi_number: 6174912
Description: PECTINESTERASE 1.1 PRECURSOR (PECTIN
METHYLESTERASE) (PE)
Seq ID: 243
Accession: P11369
Swissprot_id: POL2_MOUSE
Gi_number: 130402
Description: Retrovirus-related POL polyprotein
[Contains: Reverse transcriptase ; Endonuclease]
Seq ID: 245
Accession: P31688
Swissprot_id: TPS2_YEAST
Gi_number: 1730010
Description: TREHALOSE-PHOSPHATASE (TREHALOSE 6-
PHOSPHATE PHOSPHATASE) (TPP)
Seq ID: 247
Accession: P23586
Swissprot_id: STP1_ARATH
Gi_number: 134976
Description: GLUCOSE TRANSPORTER (SUGAR CARRIER)
Seq ID: 249
Accession: P29375
Swissprot_id: RBB2_HUMAN
Gi_number: 1710032
Description: Retinoblastoma-binding protein 2
(RBBP-2)
Seq ID: 251
Accession: P27164
Swissprot_id: CAL3_PETHY
Gi_number: 115492
Description: CALMODULIN-RELATED PROTEIN
Seq ID: 253
Accession: P74361
Swissprot_id: CLPB_SYNY3
Gi_number: 2493734
Description: ClpB protein
Seq ID: 257
Accession: P14009
Swissprot_id: 14KD_DAUCA
Gi_number: 112697
Description: 14 KD PROLINE-RICH PROTEIN DC2.15
PRECURSOR
Seq ID: 259
Accession: O46522
Swissprot_id: C24B_BOVIN
Gi_number: 6685239
Description: CYTOCHROME B-245 HEAVY CHAIN (P22
PHAGOCYTE B-CYTOCHROME) (NEUTROPHIL
CYTOCHROME B, 91 KDA POLYPEPTIDE) (CGD91-PHOX)
(GP91-PHOX)(HEME BINDING MEMBRANE GLYCOPROTEIN
GP91PHOX) (CYTOCHROME B(558) BETA CHAIN)
(SUPEROXIDE-GENERATING NADPH OXIDASE HEAVY CHA>
Seq ID: 261
Accession: P21997
Swissprot_id: SSGP_VOLCA
Gi_number: 134920
Description: SULFATED SURFACE GLYCOPROTEIN
185 (SSG 185)
Seq ID: 263
Accession: P08640
Swissprot_id: AMYH_YEAST
Gi_number: 728850
Description: GLUCOAMYLASE S1/S2 PRECURSOR
(GLUCAN 1,4-ALPHA-GLUCOSIDASE) (1,4-ALPHA-
D-GLUCAN GLUCOHYDROLASE)
Seq ID: 265
Accession: P33126
Swissprot_id: HS82_ORYSA
Gi_number: 417154
Description: HEAT SHOCK PROTEIN 82
Seq ID: 267
Accession: P27644
Swissprot_id: PGLR_AGRTU
Gi_number: 129937
Description: POLYGALACTURONASE (PECTINASE) (PGL)
Seq ID: 269
Accession: P42736
Swissprot_id: CDI3_ARATH
Gi_number: 1168862
Description: CADMIUM-INDUCED PROTEIN AS30
Seq ID: 271
Accession: P29141
Swissprot_id: SUBV_BACSU
Gi_number: 135023
Description: Minor extracellular protease VPR
precursor
Seq ID: 273
Accession: Q08275
Swissprot_id: HS23_MAIZE
Gi_number: 729762
Description: 17.0 KD CLASS II HEAT SHOCK PROTEIN
(HSP 18)
Seq ID: 275
Accession: O04701
Swissprot_id: MPC1_CYNDA
Gi_number: 14423757
Description: Major pollen allergen Cyn d 1
Seq ID: 277
Accession: P23444
Swissprot_id: H1_MAIZE
Gi_number: 121950
Description: HISTONE H1
Seq ID: 279
Accession: Q9UKN7
Swissprot_id: MY15_HUMAN
Gi_number: 13124361
Description: Myosin XV (Unconventional myosin-15)
Seq ID: 281
Accession: Q43135
Swissprot_id: C791_SORBI
Gi_number: 5915822
Description: Cytochrome P450 79A1 (Cytochrome
P450TYR)
Seq ID: 283
Accession: Q02357
Swissprot_id: ANK1_MOUSE
Gi_number: 1168457
Description: Ankyrin 1 (Erythrocyte ankyrin)
Seq ID: 285
Accession: P52914
Swissprot_id: NTPA_PEA
Gi_number: 1709358
Description: Nucleoside-triphosphatase
(Nucleoside triphosphate phosphohydrolase)
(NTPase) (Apyrase)
Seq ID: 287
Accession: Q40412
Swissprot_id: ABA2_NICPL
Gi_number: 5902707
Description: Zeaxanthin epoxidase, chloroplast
precursor
Seq ID: 289
Accession: P51074
Swissprot_id: ANX4_FRAAN
Gi_number: 1703318
Description: Annexin-like protein RJ4
Seq ID: 291
Accession: P21877
Swissprot_id: BCSA_ACEXY
Gi_number: 584832
Description: Cellulose synthase catalytic
subunit [UDP-forming]
Seq ID: 293
Accession: P24805
Swissprot_id: TSJT_TOBAC
Gi_number: 136452
Description: STEM-SPECIFIC PROTEIN TSJT1
Seq ID: 295
Accession: P18583
Swissprot_id: SON_HUMAN
Gi_number: 586013
Description: SON PROTEIN (SON3)
Seq ID: 297
Accession: P30986
Swissprot_id: RETO_ESCCA
Gi_number: 400972
Description: RETICULINE OXIDASE PRECURSOR
(BERBERINE-BRIDGE-FORMING ENZYME) (BBE)
(TETRAHYDROPROTOBERBERINE SYNTHASE)
Seq ID: 299
Accession: Q9NVW2
Swissprot_id: RNFB_HUMAN
Gi_number: 13124522
Description: RING FINGER PROTEIN 12 (LIM DOMAIN
INTERACTING RING FINGER PROTEIN) (RING FINGER
LIM DOMAIN-BINDING PROTEIN) (R-LIM) (NY-REN-43
ANTIGEN)
Seq ID: 301
Accession: P10978
Swissprot_id: POLX_TOBAC
Gi_number: 130582
Description: Retrovirus-related Pol polyprotein
from transposon TNT 1-94 [Contains: Pro tease;
Reverse transcriptase; Endonuclease]
Seq ID: 303
Accession: P29386
Swissprot_id: AGL6_ARATH
Gi_number: 1351899
Description: Agamous-like MADS box protein AGL6
Seq ID: 305
Accession: Q41819
Swissprot_id: IAAG_MAIZE
Gi_number: 2501499
Description: INDOLE-3-ACETATE BETA-
GLUCOSYLTRANSFERASE (IAA-GLU SYNTHETASE) ((URIDINE
5′-DIPHOSPHATE-GLUCOSE:INDOL-3-YLACETYL)-BETA-D-
GLUCOSYL TRANSFERASE)
Seq ID: 309
Accession: P33479
Swissprot_id: IE18_PRVKA
Gi_number: 462387
Description: IMMEDIATE-EARLY PROTEIN IE180
Seq ID: 311
Accession: P46573
Swissprot_id: APKB_ARATH
Gi_number: 12644274
Description: PROTEIN KINASE APK1B
Seq ID: 313
Accession: P00434
Swissprot_id: PERX_BRARA
Gi_number: 464365
Description: Peroxidase P7
Seq ID: 315
Accession: P28493
Swissprot_id: PR5_ARATH
Gi_number: 135915
Description: Pathogenesis-related protein 5
precursor (PR-5)
Seq ID:317
Accession: P70315
Swissprot_id: WASP_MOUSE
Gi_number: 2499130
Description: Wiskott-Aldrich syndrome protein
homolog (WASP)
Seq ID:319
Accession: P22196
Swissprot_id: PER2_ARAHY
Gi_number: 129808
Description: Cationic peroxidase 2 precursor
Seq ID:321
Accession: Q02438
Swissprot_id: E13E_HORVU
Gi_number: 1352328
Description: GLUCAN ENDO-1,3-BETA-GLUCOSIDASE
GV ((1->3)-BETA-GLUCAN ENDOHYDROLASE GV) ((1-
>3)-BETA-GLUCANASE ISOENZYME GV) (BETA-1,3-
ENDOGLUCANASE GV)
Seq ID: 323
Accession: P13652
Swissprot_id: CDD_ECOLI
Gi_number:416781
Description: Cytidine deaminase (Cytidine
aminohydrolase) (CDA)
Seq ID: 325
Accession: O48670
Swissprot_id: RERA_ARATH
Gi_number: 6225938
Description: RER1A protein (AtRER1A)
Seq ID: 327
Accession: O80340
Swissprot_id: ERF4_ARATH
Gi_number: 7531110
Description: Ethylene responsive element binding
factor 4 (AtERF4)
Seq ID: 329
Accession: Q00764
Swissprot_id: TPS1_YEAST
Gi_number: 401206
Description: ALPHA,ALPHA-TREHALOSE-PHOSPHATE
SYNTHASE [UDP-FORMING] 56 KD SUBUNIT
(TREHALOSE-6-PHOSPHATE SYNTHASE) (UDP-
GLUCOSE-GLUCOSEPHOSPHATE GLUCOSYLTRANSFERASE)
(GENERAL GLUCOSE SENSOR, SUBUNIT 1) (GLYCOGEN
METABOLISM CONTROL PROTEIN GLC6)
Seq ID: 331
Accession: P52839
Swissprot_id: FSTL_ARATH
Gi_number: 1706917
Description: Flavonol sulfotransferase-like
(RaRO47)
Seq ID: 333
Accession: P26792
Swissprot_id: INV1_DAUCA
Gi_number: 124712
Description: BETA-FRUCTOFURANOSIDASE, INSOLUBLE
ISOENZYME 1 PRECURSOR (SUCROSE-6-PHOSPHATE
HYDROLASE 1) (INVERTASE 1) (CELL WALL BETA-
FRUCTOSIDASE 1)
Seq ID: 335
Accession: P10569
Swissprot_id: MYSC_ACACA
Gi_number: 127749
Description: Myosin IC heavy chain
Seq ID:337
Accession: Q9SYQ8
Swissprot_id: CLV1_ARATH
Gi_number: 12643323
Description: RECEPTOR PROTEIN KINASE CLAVATA1
PRECURSOR
Seq ID:339
Accession: P42158
Swissprot_id: KC1D_ARATH
Gi_number: 1170622
Description: CASEIN KINASE I, DELTA ISOFORM
LIKE (CKI-DELTA)

TABLE 5
The following table illustrates the correlation between
the SEQ ID NOs of U.S. Provisional Application No. 60/368,327,
filed Mar. 27, 2002 and the SEQ ID NOs provided in the
Sequence Listing. (for example, SEQ ID NO: 1 in the Sequence
Listing corresponds to SEQ ID NO: 120535 mentioned in Provisional
Application No. 60/368,327)

| New | Previous |
|---|---|
| 1 | 120535 |
| 2 | 120536 |
| 3 | 120527 |
| 4 | 120528 |
| 5 | 149075 |
| 6 | 149076 |
| 7 | 120617 |
| 8 | 120618 |
| 9 | 149073 |
| 10 | 149074 |
| 11 | 120297 |
| 12 | 120298 |
| 13 | 121307 |
| 14 | 121308 |
| 15 | 121333 |
| 16 | 121334 |
| 17 | 121171 |
| 18 | 121172 |
| 19 | 121239 |
| 20 | 121240 |
| 21 | 121231 |
| 22 | 121232 |
| 23 | 125709 |
| 24 | 125710 |
| 25 | 120937 |
| 26 | 120938 |
| 27 | 121081 |
| 28 | 121082 |
| 29 | 120769 |
| 30 | 120770 |
| 31 | 120859 |
| 32 | 120860 |
| 33 | 149077 |
| 34 | 149078 |
| 35 | 121489 |
| 36 | 121490 |
| 37 | 121569 |
| 38 | 121570 |
| 39 | 123933 |
| 40 | 123934 |
| 41 | 124021 |
| 42 | 124022 |
| 43 | 149087 |
| 44 | 149088 |
| 45 | 125643 |
| 46 | 125644 |
| 47 | 149085 |
| 48 | 149086 |
| 49 | 123549 |
| 50 | 123550 |
| 51 | 123699 |
| 52 | 123700 |
| 53 | 123669 |
| 54 | 123670 |
| 55 | 123275 |
| 56 | 123276 |
| 57 | 123361 |
| 58 | 123362 |
| 59 | 123373 |
| 60 | 123374 |
| 61 | 123261 |
| 62 | 123262 |
| 63 | 123435 |
| 64 | 123436 |
| 65 | 125635 |
| 66 | 125636 |
| 67 | 123403 |
| 68 | 123404 |
| 69 | 123387 |
| 70 | 123388 |
| 71 | 149089 |
| 72 | 149090 |
| 73 | 124257 |
| 74 | 124258 |
| 75 | 124235 |
| 76 | 124236 |
| 77 | 124961 |
| 78 | 124962 |
| 79 | 124947 |
| 80 | 124948 |
| 81 | 124929 |
| 82 | 124930 |
| 83 | 124921 |
| 84 | 124922 |
| 85 | 125089 |
| 86 | 125090 |
| 87 | 125075 |
| 88 | 125076 |
| 89 | 125053 |
| 90 | 125054 |
| 91 | 125133 |
| 92 | 125134 |
| 93 | 125121 |
| 94 | 125122 |
| 95 | 125535 |
| 96 | 125536 |
| 97 | 124687 |
| 98 | 124688 |
| 99 | 124811 |
| 100 | 124812 |
| 101 | 124849 |
| 102 | 124850 |
| 103 | 149091 |
| 104 | 149092 |
| 105 | 124489 |
| 106 | 124490 |
| 107 | 124529 |
| 108 | 124530 |
| 109 | 125477 |
| 110 | 125478 |
| 111 | 125381 |
| 112 | 125382 |
| 113 | 125203 |
| 114 | 125204 |
| 115 | 122605 |
| 116 | 122606 |
| 117 | 122911 |
| 118 | 122912 |
| 119 | 149079 |
| 120 | 149080 |
| 121 | 122771 |
| 122 | 122772 |
| 123 | 122527 |
| 124 | 122528 |
| 125 | 149083 |
| 126 | 149084 |
| 127 | 123157 |
| 128 | 123158 |
| 129 | 121699 |
| 130 | 121700 |
| 131 | 122215 |
| 132 | 122216 |
| 133 | 122847 |
| 134 | 122848 |
| 135 | 121929 |
| 136 | 121930 |
| 137 | 149081 |
| 138 | 149082 |
| 139 | 121749 |
| 140 | 121750 |
| 141 | 122161 |
| 142 | 122162 |
| 143 | 121997 |
| 144 | 121998 |
| 145 | 149001 |
| 146 | 149002 |
| 147 | 149003 |
| 148 | 149004 |
| 149 | 5893 |
| 150 | 5894 |
| 151 | 6991 |
| 152 | 6992 |
| 153 | 7619 |
| 154 | 7620 |
| 155 | 10213 |
| 156 | 10214 |
| 157 | 14223 |
| 158 | 14224 |
| 159 | 149005 |
| 160 | 149006 |
| 161 | 17655 |
| 162 | 17656 |
| 163 | 18629 |
| 164 | 18630 |
| 165 | 22941 |
| 166 | 22942 |
| 167 | 22945 |
| 168 | 22946 |
| 169 | 25033 |
| 170 | 25034 |
| 171 | 27301 |
| 172 | 27302 |
| 173 | 27689 |
| 174 | 27690 |
| 175 | 27725 |
| 176 | 27726 |
| 177 | 28059 |
| 178 | 28060 |
| 179 | 149051 |
| 180 | 149052 |
| 181 | 149007 |
| 182 | 149008 |
| 183 | 29425 |
| 184 | 29426 |
| 185 | 30813 |
| 186 | 30814 |
| 187 | 31907 |
| 188 | 31908 |
| 189 | 32423 |
| 190 | 32424 |
| 191 | 33249 |
| 192 | 33250 |
| 193 | 149009 |
| 194 | 149010 |
| 195 | 149053 |
| 196 | 149054 |
| 197 | 149055 |
| 198 | 149056 |
| 199 | 149011 |
| 200 | 149012 |
| 201 | 149013 |
| 202 | 149014 |
| 203 | 149015 |
| 204 | 149016 |
| 205 | 38911 |
| 206 | 38912 |
| 207 | 41457 |
| 208 | 41458 |
| 209 | 41905 |
| 210 | 41906 |
| 211 | 149017 |
| 212 | 149018 |
| 213 | 44427 |
| 214 | 44428 |
| 215 | 149019 |
| 216 | 149020 |
| 217 | 149057 |
| 218 | 149058 |
| 219 | 47197 |
| 220 | 47198 |
| 221 | 47475 |
| 222 | 47476 |
| 223 | 47585 |
| 224 | 47586 |
| 225 | 149021 |
| 226 | 149022 |
| 227 | 50851 |
| 228 | 50852 |
| 229 | 149023 |
| 230 | 149024 |
| 231 | 149025 |
| 232 | 149026 |
| 233 | 52307 |
| 234 | 52308 |
| 235 | 53513 |
| 236 | 53514 |
| 237 | 53973 |
| 238 | 53974 |
| 239 | 56253 |
| 240 | 56254 |
| 241 | 149059 |
| 242 | 149060 |
| 243 | 149061 |
| 244 | 149062 |
| 245 | 58923 |
| 246 | 58924 |
| 247 | 59651 |
| 248 | 59652 |
| 249 | 149027 |
| 250 | 149028 |
| 251 | 61707 |
| 252 | 61708 |
| 253 | 62633 |
| 254 | 62634 |
| 255 | 149063 |
| 256 | 149064 |
| 257 | 149029 |
| 258 | 149030 |
| 259 | 68781 |
| 260 | 68782 |
| 261 | 70307 |
| 262 | 70308 |
| 263 | 149031 |
| 264 | 149032 |
| 265 | 74071 |
| 266 | 74072 |
| 267 | 74767 |
| 268 | 74768 |
| 269 | 76351 |
| 270 | 76352 |
| 271 | 77599 |
| 272 | 77600 |
| 273 | 149033 |
| 274 | 149034 |
| 275 | 78959 |
| 276 | 78960 |
| 277 | 80875 |
| 278 | 80876 |
| 279 | 149035 |
| 280 | 149036 |
| 281 | 83163 |
| 282 | 83164 |
| 283 | 149065 |
| 284 | 149066 |
| 285 | 149037 |
| 286 | 149038 |
| 287 | 85455 |
| 288 | 85456 |
| 289 | 149067 |
| 290 | 149068 |
| 291 | 149039 |
| 292 | 149040 |
| 293 | 90315 |
| 294 | 90316 |
| 295 | 90359 |
| 296 | 90360 |
| 297 | 149041 |
| 298 | 149042 |
| 299 | 95133 |
| 300 | 95134 |
| 301 | 149069 |
| 302 | 149070 |
| 303 | 95635 |
| 304 | 95636 |
| 305 | 149071 |
| 306 | 149072 |
| 307 | 97077 |
| 308 | 97078 |
| 309 | 98203 |
| 310 | 98204 |
| 311 | 98309 |
| 312 | 98310 |
| 313 | 149043 |
| 314 | 149044 |
| 315 | 99619 |
| 316 | 99620 |
| 317 | 149045 |
| 318 | 149046 |
| 319 | 100527 |
| 320 | 100528 |
| 321 | 149047 |
| 322 | 149048 |
| 323 | 101447 |
| 324 | 101448 |
| 325 | 101953 |
| 326 | 101954 |
| 327 | 102459 |
| 328 | 102460 |
| 329 | 108545 |
| 330 | 108546 |
| 331 | 149049 |
| 332 | 149050 |
| 333 | 109193 |
| 334 | 109194 |
| 335 | 109503 |
| 336 | 109504 |
| 337 | 110137 |
| 338 | 110138 |
| 339 | 110281 |
| 340 | 110282 |
| 341 | 110823 |
| 342 | 110824 |

TABLE 6
this table provides SEQ ID NOs of banana, wheat, rice
and maize representing cDNA/EST sequences that are homologous
to the rice sequences show in column 1.

| Rice | Banan | Wheat | Rice | Maize |
|---|---|---|---|---|
| (SEQ ID) | (SEQ ID) | (SEQ ID) | (SEQ ID) | (SEQ ID) |
| 1 | 535 | 658 | 355 | 756 |
| 3 | 534 | 594 | 357 | 688 |
| 5 | 508 | 566 | 447 | 709 |
| 7 | 474 | 626 | 449 | 758 |
| 9 | — | 569 | 343 | 757 |
| 11 | 518 | 587 | | 702 |
| 15 | — | 604 | 359 | 704 |
| 17 | — | 608 | 369 | 694 |
| 19 | — | 650 | 381 | 711 |
| 21 | 518 | 589 | 361 | 683 |
| 23 | 530 | 600 | 445 | 765 |
| 25 | 521 | 646 | 363 | 712 |
| 27 | 527 | — | 373 | 733 |
| 29 | 495 | 554 | 379 | 684 |
| 31 | 484 | 659 | 375 | — |
| 33 | — | 641 | 383 | 737 |
| 35 | — | 597 | | 715 |
| 37 | 531 | 639 | 377 | 736 |
| 39 | 488 | 655 | 367 | 752 |
| 41 | 532 | 571 | 389 | 682 |
| 43 | — | 614 | 391 | 776 |
| 45 | 538 | 651 | 415 | 713 |
| 47 | 491 | 584 | 397 | 698 |
| 49 | 504 | 636 | 399 | 729 |
| 51 | 492 | 673 | 405 | 734 |
| 53 | 525 | 669 | 393 | 779 |
| 55 | — | 644 | 401 | — |
| 57 | 501 | 672 | 407 | 728 |
| 59 | 515 | 592 | 395 | 747 |
| 61 | — | 617 | 403 | 747 |
| 63 | 513 | 649 | | 742 |
| 65 | — | 638 | 385 | 725 |
| 67 | 511 | 595 | 413 | 720 |
| 69 | 505 | 593 | 411 | 741 |
| 71 | 502 | 620 | 409 | 762 |
| 73 | — | 567 | | — |
| 75 | 510 | 666 | 429 | 773 |
| 77 | 517 | 579 | 423 | 771 |
| 79 | 480 | 624 | 419 | 755 |
| 81 | — | — | 417 | 692 |
| 83 | 489 | 631 | 387 | 746 |
| 85 | 530 | 627 | 345 | 774 |
| 87 | — | 633 | 435 | — |
| 89 | 523 | 613 | 347 | 726 |
| 91 | 536 | 596 | 371 | 691 |
| 93 | 528 | 642 | | 735 |
| 95 | 533 | 602 | 351 | 750 |
| 97 | 529 | 607 | 425 | 732 |
| 99 | — | 640 | 431 | 677 |
| 101 | 490 | 565 | 433 | 743 |
| 103 | 485 | 622 | 437 | 778 |
| 105 | 531 | 612 | 439 | 731 |
| 107 | 497 | 581 | 441 | 723 |
| 109 | 532 | 571 | 353 | 682 |
| 111 | 524 | 603 | 427 | 745 |
| 113 | — | 560 | 443 | 697 |
| 115 | — | 599 | | 753 |
| 117 | 512 | 611 | 451 | 695 |
| 121 | 514 | 635 | 453 | 760 |
| 123 | — | 577 | 455 | 777 |
| 125 | 493 | 670 | 457 | 710 |
| 127 | — | 543 | 459 | 699 |
| 129 | 481 | 632 | 461 | 748 |
| 131 | — | 545 | | 763 |
| 133 | — | 578 | 463 | — |
| 135 | — | 668 | | — |
| 137 | 521 | 606 | 465 | 693 |
| 139 | 503 | 588 | 467 | 708 |
| 141 | 487 | 648 | 469 | — |
| 143 | 494 | 580 | 471 | — |
| 145 | — | 568 | | 675 |
| 147 | 496 | 665 | | — |
| 149 | — | 540 | | 707 |
| 151 | 487 | 546 | | — |
| 153 | 494 | 580 | | — |
| 155 | — | 623 | | 703 |
| 159 | 504 | 636 | | 729 |
| 161 | 538 | 582 | | 775 |
| 163 | — | 653 | | — |
| 165 | — | 633 | | — |
| 167 | 522 | 548 | | 714 |
| 169 | 509 | 557 | | — |
| 171 | — | 641 | 383 | 737 |
| 173 | 530 | 627 | | 774 |
| 175 | — | 591 | | 716 |
| 177 | 530 | 600 | 445 | 765 |
| 179 | — | 550 | | — |
| 181 | — | 552 | | 768 |
| 183 | 516 | 562 | | 727 |
| 185 | 477 | 654 | | 686 |
| 187 | — | 577 | 455 | 111 |
| 189 | — | 667 | | — |
| 191 | 527 | 553 | | 733 |
| 193 | — | 664 | | 770 |
| 195 | 513 | 559 | | — |
| 197 | — | 621 | | 764 |
| 199 | — | 650 | 421 | 711 |
| 201 | 483 | 570 | | 700 |
| 203 | — | 601 | | — |
| 205 | 474 | 626 | | 758 |
| 207 | 505 | 590 | | 722 |
| 209 | 482 | 661 | | 706 |
| 211 | 502 | 620 | | 762 |
| 213 | 529 | 607 | 349 | 732 |
| 215 | — | 558 | | 753 |
| 217 | 512 | 611 | | 695 |
| 219 | — | 615 | | 696 |
| 221 | 531 | 671 | | 751 |
| 223 | 533 | 625 | | 749 |
| 225 | 511 | 616 | | 676 |
| 229 | 532 | 630 | | 719 |
| 231 | — | 610 | | 737 |
| 233 | — | 583 | | 769 |
| 235 | — | 575 | | — |
| 237 | — | 656 | | 717 |
| 239 | 526 | 643 | | 679 |
| 241 | 508 | 566 | | 709 |
| 245 | 478 | 561 | | 738 |
| 247 | 479 | 609 | | 689 |
| 249 | 500 | 657 | | — |
| 251 | 534 | 594 | | 688 |
| 253 | — | 551 | | 761 |
| 257 | 521 | 646 | 363 | 712 |
| 259 | — | 541 | | 680 |
| 261 | 491 | 584 | | 698 |
| 263 | — | 638 | 385 | 725 |
| 265 | 486 | 618 | | 746 |
| 267 | 480 | 624 | | 755 |
| 269 | — | 662 | | 759 |
| 271 | — | 556 | | — |
| 273 | 523 | 613 | | 726 |
| 275 | 473 | 652 | | 739 |
| 277 | 536 | 596 | 371 | 691 |
| 279 | 517 | 605 | | 685 |
| 281 | — | 544 | | — |
| 283 | — | 573 | | — |
| 285 | 498 | 564 | | 681 |
| 287 | 525 | 669 | | 779 |
| 289 | — | 644 | 401 | — |
| 291 | 506 | — | | 678 |
| 293 | 485 | 622 | 365 | 778 |
| 295 | 514 | 572 | | 690 |
| 297 | — | 574 | | 766 |
| 299 | — | 598 | | 754 |
| 301 | — | 547 | | — |
| 303 | 539 | 585 | | 721 |
| 305 | 476 | 660 | | 740 |
| 307 | — | 549 | | 705 |
| 309 | 537 | 629 | | 684 |
| 311 | 475 | 628 | | 687 |
| 313 | — | 619 | 403 | 747 |
| 315 | — | 555 | | 744 |
| 317 | — | 672 | | 728 |
| 319 | 519 | 604 | | 718 |
| 321 | 531 | 639 | 377 | 736 |
| 323 | — | 645 | | 701 |
| 325 | 493 | 647 | | 710 |
| 327 | — | 588 | | 730 |
| 329 | 499 | 586 | | 692 |
| 331 | — | 563 | | — |
| 333 | — | 637 | | 767 |
| 335 | — | 663 | | 724 |
| 337 | 520 | 634 | | 772 |
| 339 | 507 | 576 | | 674 |
| 341 | — | 542 | | — |

What is claimed is:

1. An isolated polynucleotide comprising a plant nucleotide sequence
(a) selected from the group consisting of SEQ ID NOs: 1 to 341 or a fragment thereof which encodes a partial-length polypeptide having substantially the same activity as the full-length polypeptide;
(b) having substantial similarity to (a);
(c) having at least 15 nucleotides and capable of hybridizing to (a) or the complement thereof under stringent conditions;
(d) having at least 15 nucleotides and capable of hybridizing to a nucleic acid comprising 50 to 200 or more consecutive nucleotides of a nucleotide sequence given in SEQ ID NOs: 1 to 341 or the complement thereof under stringent conditions;
(e) complementary to (a), (b) or (c); or
(f) which is a reverse complement of (a), (b) or (c).

2. An isolated polynucleotide comprising a nucleotide sequence having at least 70% nucleic acid sequence identity with the polynucleotide of claim 1 or the complement thereof.

3. An isolated polynucleotide comprising a nucleic acid sequence having at least 80% nucleic acid sequence identity with the polynucleotide of claim 1 or the complement thereof.

4. An isolated polynucleotide comprising a nucleic acid sequence having at least 90% nucleic acid sequence identity with the polynucleotide of claim 1 or the complement thereof.

5. An isolated polynucleotide comprising a nucleic acid sequence having at least 98% nucleic acid sequence identity with the polynucleotide of claim 1 or the complement thereof.

6. The polynucleotide of claim 1, wherein the polynucleotide comprises a plant nucleotide sequence encoding a polypeptide which modulates gene expression through posttranscriptional gene silencing.

7. The polynucleotide of claim 6, wherein the polynucleotide comprises a plant nucleotide sequence encoding a polypeptide that is substantially similar to a polypeptide with a amino acid sequence selected from the group consisting of SEQ ID NOs: 2 to 342, or a fragment thereof which has substantially the same activity as the full-length polypeptide.

8. A vector comprising the polynucleotide of claim 1.

9. An expression cassette comprising the polynucleotide of claim 1, operably linked to a regulatory sequence.

10. The expression cassette of claim 9, wherein the regulatory sequence is selected from the group consisting of a promoter, an operator, an enhancer, a repressor binding site and a transcription factor binding site.

11. The expression cassette of claim 9, wherein the polynucleic acid segment is oriented relative to the promoter such that an antisense message is transcribed.

12. A cell comprising the expression cassette of claim 9.

13. The cell of claim 12, wherein the cell is a plant cell.

14. The cell of claim 12, wherein the cell is a monocotyledonous cell, a dicotyledonous cell, a cereal plant cell, a Rosidea cell, a Brassicales cell, an Arabidopsis cell, a rice plant cell, a wheat plant cell, a barley plant cell or a maize plant cell.

15. A recombinant cell, wherein the genome of the cell is augmented with the expression cassette of claim 9.

16. The recombinant cell of claim 15, wherein the cell is a plant cell.

17. A mutagenesis cassette comprising an intervening nucleic acid sequence linked on both ends to a flanking nucleic acid sequence that hybridizes under low stringency conditions to a nucleic acid sequence selected from the group consisting of SEQ ID NOs: 1 to 341, or a complement thereof.

18. The mutagenesis cassette of claim 17, wherein the intervening nucleic acid sequence is a selectable marker.

19. A vector comprising the mutagenesis cassette of claim 17.

20. A polypeptide comprising an amino acid sequence encoded by a nucleic acid sequence having at least 70% sequence identity with a nucleic acid sequence selected from the group consisting of SEQ ID NOs: 1 to 341 or a complement thereof.

21. A polypeptide comprising an amino acid sequence encoded by a nucleic acid sequence having at least 80% sequence identity with a nucleic acid sequence selected from the group consisting of SEQ ID NOs: 1 to 341, or a complement thereof.

22. A polypeptide comprising an amino acid sequence encoded by a nucleic acid sequence having at least 90% sequence identity with a nucleic acid sequence selected from the group consisting of SEQ ID NOs: 1 to 341, or a complement thereof.

23. A polypeptide comprising an amino acid sequence encoded by a nucleic acid sequence having at least 98% sequence identity with a nucleic acid sequence selected from the group consisting of SEQ ID NOs: 1 to 341, or a complement thereof.

24. The polypeptide of claim 20, wherein the polypeptide modulates gene expression by posttranscriptional gene silencing.

25. A method to modulate gene expression within a cell by posttranscriptional gene silencing comprising:
a) introducing into a cell a polynucleotide having at least 70% nucleic acid sequence identity with a nucleic acid sequence selected from the group consisting of SEQ ID NOs: 1 to 341, or a complement thereof, operably linked to a regulatory sequence; and
(b) expressing the polynucleotide in the cell to form a product, wherein the product modulates gene expression within the cell.

26. The method of claim 25, wherein the cell is a monocotyledonous cell, a dicotyledonous cell, a cereal plant cell, a Rosidea cell, a Brassicales cell, an Arabidopsis cell, a rice plant cell, a wheat plant cell, a barley plant cell or a maize plant cell.

27. The method of claim 25, wherein the regulatory sequence is selected from the group consisting of a promoter, an operator, an enhancer, a repressor binding site and a transcription factor binding site.

28. A method for isolating a polynucleic acid fragment containing a regulatory element that modulates expression of a polynucleic acid segment within a cell by posttranscriptional gene silencing, comprising:
a) hybridizing an oligonucleotide probe, wherein said oligonucleotide probe hybridizes to a polynucleic acid segment having at least 70% nucleic acid sequence identity with a nucleic acid sequence selected from the group consisting of SEQ ID NOs: 1 to 341 and having expression that is modulated within a cell by posttranscriptional gene silencing, to a polynucleic acid fragment that corresponds to genomic DNA obtained from the cell to form a complex, wherein the polynucleic acid fragment comprises the regulatory element and the polynucleic acid segment; and
b) isolating the polynucleic acid fragment from the complex formed by hybridization of the oligonucleotide to the polynucleic acid fragment containing the regulatory element.

29. The method of claim 28, further comprising sequencing the isolated polynucleic acid fragment.

30. The method of claim 28, wherein the oligonucleotide probe hybridizes under low stringency conditions to a polynucleic acid segment selected from the group consisting of SEQ ID NOs: 1 to 341, or a complement thereof.

31. The method of claim 28, wherein the cell is a plant cell.

32. A method for isolating the regulatory element from the polynucleic acid fragment of claim 28, comprising:
a) screening for expression of a detectable marker in cells transformed with a vector that encodes the detectable marker and contains a portion of the polynucleic acid fragment, wherein the portion of the polynucleic acid fragment causes expression of the detectable marker to be modulated by posttranscriptional gene silencing; and
b) isolating the regulatory element from the vector obtained from the cell wherein expression of the detectable marker is modulated by posttranscriptional gene silencing.

33. A regulatory element isolated according to the method of claim 32.

34. A method for obtaining an amplification product containing a regulatory element that modulates expression of a polynucleic acid segment in a cell by posttranscriptional gene silencing, comprising:
a) amplifying a nucleic acid sequence that contains the regulatory element by hybridizing a first oligonucleotide primer to a polynucleic acid fragment comprising the polynucleic acid segment and the regulatory element, and hybridizing a second degenerate oligonucleotide primer to the polynucleic acid fragment at a position that is 5′ or 3′ to an open reading frame containing the polynucleic acid segment, and amplifying the nucleic acid sequence between the first primer and the second primer by polymerase chain reaction to form an amplification product, wherein the polynucleic acid fragment is genomic DNA from the cell; and
b) isolating the amplification product formed by the polymerase chain reaction between the first and the second primer to obtain the regulatory element.

35. The method of claim 34, further comprising sequencing the amplification product.

36. The method of claim 34, wherein the first oligonucleotide primer hybridizes to a nucleic acid segment selected from the group consisting of SEQ ID NOs: 1 to 341, or a complement thereof.

37. An amplification product isolated according to the method of claim 34.

38. A method for isolating the regulatory element of claim 34 from the amplification product comprising:
a) screening for expression of a detectable marker in cells that were transformed with a vector that encodes the detectable marker and contains a portion of the amplification product, wherein the portion of the amplification product causes expression of the detectable marker to be modulated by posttranscriptional gene silencing; and
b) isolating the regulatory element from the vector obtained from the cell wherein expression of the detectable marker is modulated by posttranscriptional gene silencing.

39. A regulatory element isolated according to the method of claim 38.

40. An expression cassette comprising the regulatory element of claim 33 operably linked to an open reading frame.

41. An expression cassette comprising the regulatory element of claim 39 operably linked to an open reading frame.

42. A method to augment a plant genome comprising:
a) contacting a plant cell with the polynucleotide of claim 1 to produce a transformed plant cell; and
b) growing the transformed plant cell to produce a differentiated transformed plant.

43. The method of claim 42, wherein the plant cell is selected from the group consisting of a monocotyledonous cell, a dicotyledonous cell, a cereal plant cell, a wheat plant cell, a rice plant cell, a barley cell, a maize plant cell, a Rosidea cell, a Brassicales cell, and an Arabidopsis cell.

44. A transgenic plant comprising a polynucleotide of claim 1.

45. A transgenic plant comprising the polynucleotide of claim 2.

46. The plant of claim 45, wherein the plant is a monocotyledonous plant, a dicotyledonous plant, a cereal plant, a wheat plant, a rice plant, a barley plant, a maize plant, a Rosidea plant, a Brassicales plant or an Arabidopsis plant.

47. A product produced by a plant of claim 45, wherein the product is a seed, a fruit, a vegetable, a transgenic plant, a progeny plant or products of the progeny plant.

48. A method to create a mutant cell comprising: contacting a cell with the mutagenesis cassette of claim 18 to produce a cell having a deletion in a gene that is regulated by posttranscriptional gene silencing.

49. The method of claim 48, wherein the cell is a plant cell.

50. A method for identifying a first polypeptide that forms a complex with a second polypeptide within a cell, comprising:
a) expressing within the cell the second polypeptide, wherein said second polypeptide is a fusion polypeptide that comprises a marker polypeptide and a polypeptide having expression that is modulated by posttranscriptional gene silencing, so that a complex is formed that contains the first polypeptide and the second polypeptide; and
b) separating the complex by using the marker polypeptide and identifying the first polypeptide contained within the complex.

51. The method of claim 50, wherein the second polypeptide is encoded by a polynucleic acid sequence selected from the group consisting of SEQ ID NOs: 1 to 341, or a complement thereof.

52. The method of claim 50, wherein the marker polypeptide is glutathione-S-transferase.

53. The method of claim 50, wherein the marker polypeptide is an epitope for an antibody.

54. A method for determining a polynucleotide that encodes a first polypeptide that interacts with a second polypeptide whose expression is modulated by posttranscriptional gene silencing, comprising:
a) mating a first haploid yeast cell, wherein said first haploid yeast cell contains a construct comprising a polynucleotide that encodes the first polypeptide fused to a DNA-binding domain or to an RNA polymerase activation domain, with a second haploid yeast cell, which contains a construct comprising a nucleic acid sequence selected from SEQ ID NOs: 1 to 341 fused to a DNA-binding domain or an RNA polymerase activation domain, wherein the first haploid yeast strain and the second haploid yeast strain do not contain a construct that encodes for the same DNA-binding domain or RNA-polymerase activation domain and either of the first or the second haploid yeast strain comprises a two-hybrid reporter gene; and
b) determining the sequence that encodes the first polypeptide by sequencing the construct that expresses the first polypeptide as indicated by measuring the activation of the two-hybrid reporter gene in the diploid yeast strain.

55. An isolated polynucleotide segment that hybridizes under low stringency conditions to a nucleic acid sequence selected from the group consisting of SEQ ID NOs: 1 to 341, or a complement thereof.

56. An isolated polynucleotide segment that encodes a polypeptide having substantial similarity to a polypeptide encoded by a nucleic acid sequence selected from the group consisting of SEQ ID NOs: 1 to 341, or a complement thereof.

57. An isolated polynucleotide segment having a similarity value less than 1×10⁻⁵ as determined by BLAST search using default parameters to a nucleic acid sequence selected from the group consisting of SEQ ID NOs: 1 to 341, or a complement thereof.

58. An isolated polynucleotide segment having a similarity value less than 1×10⁻¹⁰ as determined by BLAST search using default parameters to a nucleic acid sequence selected from the group consisting of SEQ ID NOs: 1 to 341, or a complement thereof.

59. An isolated polynucleotide segment having a similarity value less than 1×10⁻²⁰ as determined by BLAST search using default parameters to a nucleic acid sequence selected from the group consisting of SEQ ID NOs: 1 to 341, or a complement thereof.

60. An isolated polynucleotide segment having a similarity value less than 1×10⁻²⁵ as determined by BLAST search using default parameters to a nucleic acid sequence selected from the group consisting of SEQ ID NOs: 1 to 341, or a complement thereof.

61. A cell comprising the isolated polynucleotide segment of claim 57.

62. A transgenic plant comprising the polynucleic acid segment of claim 57.

63. A computer-readable medium having stored thereon a data structure comprising:
a) at least one nucleic acid sequence that encodes a polypeptide which confers post-transcriptional gene silencing onto a plant and has at least 70% nucleic acid sequence identity to a nucleic acid sequence selected from the group consisting of SEQ ID NOs: 1 to 341, or a complement thereof; and
b) a module receiving the nucleic acid sequence which compares the nucleic acid sequence to another nucleic acid sequence.

64. The computer readable medium of claim 63, wherein the medium is selected from the group consisting of magnetic tape, optical disk, CD-ROM, random access memory, volatile memory, non-volatile memory and bubble memory.

65. A computer-readable medium having stored thereon computer executable instructions for performing a method comprising:
a) receiving at least one nucleic acid sequence having at least 70% nucleic acid sequence identity to a nucleic acid sequence selected from the group consisting of SEQ ID NOs: 1 to 341, 473 to 539; 540 to 673, and 674 to 779, or a complement thereof; and
b) comparing the nucleic acid sequence to another nucleic acid sequence.

66. The computer readable medium of claim 65, wherein the medium is selected from the group consisting of magnetic tape, optical disk, CD-ROM, random access memory, volatile memory, non-volatile memory and bubble memory.

67. A method of determining the silencing status of a plant comprising
(a) obtaining an RNA expression profile for a subset of genes comprising one or more genes the expression of which is modulated by posttranscriptional gene silencing (PTGS); and
(b) comparing the expression profile obtained with the profile of a plant of the same species that does not have posttranscriptional gene silencing.
